(19) United States
(12) Patent Application Publication
(10) Pub. No.: US 2021/0231542 A1
(43) Pub. Date: Jul. 29, 2021

(54) METHOD AND DEVICE FOR CONTROLLING POSITIONING OF PATCH

(71) Applicant: NOUL CO., LTD., Yongin-si Gyeonggi-do (KR)

(72) Inventors: Young Hoon SONG, Yongin-si, Gyeonggi-do (KR); Dong Young LEE, Yongin-si, Gyeonggi-do (KR); Jae Ryun CHO, Suwon-si, Gyeonggi-do (KR)

(21) Appl. No.: 17/052,415

(22) PCT Filed: Apr. 3, 2019
(86) PCT No.: PCT/KR2019/003930

§ 371 Date: Nov. 2, 2020

(30) Foreign Application Priority Data
May 3, 2018 (KR) 10-2018-0051322

(51) Int. Cl. G01N 1/31; G01N 35/00

(52) U.S. Cl. CPC G01N 1/312; G01N 2035/00138; G01N 35/00584; G01N 35/00029

(57) ABSTRACT

Provided is a method of controlling the posture of a patch for staining a specimen by using the patch. The method includes positioning the patch to be in a first posture, which is an oblique posture, allowing the patch to access toward the surface of the substrate on which the specimen is located while the patch retains the first posture thereof, and bringing the patch to be in contact with the specimen placed on the substrate to deliver the staining reagent to the specimen.

### TECHNICAL FIELD

The present disclosure relates to a method of controlling the posture of a gel-type patch that contains staining reagents. Specifically, the present disclosure relates to a patch posture control method for staining a specimen by using a gel-type patch.

### BACKGROUND ART

With the rapidly increasing aging population and the increasing desire for quality of life, the diagnostic market for early diagnosis and early treatment has grown every year in the world including Korea. In this background, quick and simple diagnosis is emerging as an important issue. In particular, the diagnosis method has been changed into a diagnosis without using large diagnostic equipment, such as an in-vitro diagnosis (IVD) or a point-of-care testing (POCT), which is carried out right next to the patient. Meanwhile, immunochemical diagnosis, as an example of IVD, occupies a large proportion in the field of in vitro diagnosis, and is one of the widely used diagnosis methods.

Immunochemical diagnosis, collectively referring to diagnosis using clinical immunological analysis and chemical analysis, uses an antigen-antibody reaction, and is used for diagnosis and tracking of various diseases such as various diseases including allergies or cancer markers. Due to the diversity of detectable diseases and ease of detection, the immunochemical diagnosis is evaluated as a diagnosis type particularly suitable for on-site diagnosis. The demand for the immunochemical diagnosis is steadily increasing worldwide, especially in China.

In an immunodiagnosis method of the related art, in the process of detecting an antigen that causes a disease to be diagnosed by injecting an antibody into a sample, a washing treatment is required to remove antibodies that do not bind to the target antigen or remove other factors that interfere with detection. The washing treatment is a process in which a great amount of washing solution is poured to rinse, for example, a plate. In this regard, the amount of washing solution consumed is great. In addition, in the immunodiagnosis method of the related art, in order to increase the effective contact surface area between the immobilized antibody and the applied antigen, a separate effort needs to be made for the design of the area to which the antibody is applied. Also, the complicated designed area affects the detection of a reaction.

Accordingly, there is a need to develop a delivery method for evenly delivering reagents to samples while minimizing the amount of reagents required for diagnosis. In addition, it is required to apply a method to minimize damage to the sample during the delivery of the reagent.

In addition, in order to improve the accuracy of the test, it is required to apply a procedure for effectively removing, from the sample, components that interfere with detection and reaction residues.

### DESCRIPTION OF EMBODIMENTS

### Technical Problem

The objective of the present disclosure is to provide a patch that contains a substance.

An objective of the present disclosure is to provide a patch that provides a reaction space for the substance.

The objective of the present disclosure is to provide a patch that delivers a substance.

The objective of the present disclosure is to provide a patch that absorbs a substance.

An objective of the present disclosure is to provide a patch that provides an environment.

An objective of the present disclosure is to provide a patch that contains an antibody.

An objective of the present disclosure is to provide a method of testing a sample by using a patch.

An objective of the present disclosure is to provide a device for testing a sample by using a patch.

An objective of the present disclosure is to provide a method of controlling a patch to test a sample by using a patch.

An objective of the present disclosure is to provide a device for controlling a patch to test a sample by using a patch.

### Solution to Problem

An aspect of the present disclosure provides a method of controlling the posture of a patch by using a gel-type patch which includes a staining reagent used to stain a specimen, a net structure forming micro-cavities containing the staining reagent, and a contact surface which is brought in contact with the specimen to deliver the staining reagent.

The patch posture control device may include positioning the patch to be in a first posture, which is an oblique posture at which an end of the contact surface is closer to the surface of the substrate on which the specimen is placed than another end of the contact surface while the contact surface of the patch faces the surface of the substrate on which the specimen is placed, and the patch posture control device may include allowing the patch to access toward the surface of the substrate while the patch retains the first posture, and bringing the patch to be in contact with the specimen placed on the surface of the substrate so as to deliver the staining reagent to the specimen.

The bringing the patch to be in contact with the specimen may include bringing an end of the patch to be in contact with the specimen placed on the surface of the substrate while the patch retains the first posture, and changing the posture of the patch from the first posture to the second posture at which the patch is parallel to the surface of the substrate so as to extend the contact area in which the patch contacts the specimen extends in the first direction while the contact surface of the patch is fitted with the surface of the substrate.

An another aspect of the present disclosure provides a device of controlling the posture of a patch to stain a specimen by using a gel-type patch which includes a staining reagent used to stain a specimen, a net structure forming micro-cavities containing the staining reagent, and a contact surface which is brought in contact with the specimen to deliver the staining reagent.

The patch posture control device may include a substrate fixing unit for fixing a substrate on which the specimen is located, a kit receiving unit in which a kit is located, wherein the kit includes at least one patch receiving member for receiving the patch such that at least a portion of the contact surface is exposed, and a control unit to control the position of the patch receiving member relative to the substrate.

The control unit may position the patch to be in a first posture, which is an oblique posture at which an end of the contact surface is closer to the surface of the substrate on which the specimen is placed than another end of the contact surface while the contact surface of the patch faces the surface of the substrate on which the specimen is placed, allow the patch to access the surface of the substrate while maintaining the patch to be at the first posture, and bring the patch to be in contact with the specimen placed on the surface of the substrate to deliver the staining reagent to the specimen.

A method of controlling a patch according to another aspect of the present disclosure may include bringing one side of a patch to be in contact with a specimen and sequentially bringing a contact surface to be in contact with the specimen from one side to another side of the contact surface to prevent the formation of bubbles between the contact surface and the specimen, delivering the staining reagent to the specimen through the contact surface of the patch which is in contact with the specimen, and sequentially separating the contact surface of the patch from the specimen from the side to the other side of the contact surface to prevent the deformation of the specimen.

A patch control device according to another aspect of the present disclosure includes a substrate fixing unit to fix the substrate on which the specimen is located, a kit including at least one patch receiving member, which receives the patch, such that at least a portion of the contact surface is exposed to the outside, and a control unit to control the posture of the at least one patch receiving member relative to the substrate, wherein the control unit may control the posture of the patch receiving member such that one side of the patch is brought in contact with the specimen, the contact surface is sequentially brought in contact with the specimen from one side to the other of the patch to prevent the formation of air bubbles between the contact surface and the specimen, the staining reagent is delivered to the specimen through the contact surface of the patch, and the contact surface of the patch is sequentially separated from one side to the other side of the patch to uniformly deliver the staining reagent to the area in which the specimen is distributed.

A method of controlling a patch according to another aspect of the present disclosure may include placing the patch in a first posture at which the contact surface of the patch faces the surface of the substrate on which the specimen is placed on one side of the surface of the substrate, allowing the patch to access toward the surface of the substrate while the patch retains the first posture thereof, changing the posture of the patch from the first posture to a second posture, in which at least a portion of the contact surface contacts the specimen, to bring the patch to be in contact with the specimen, thereby delivering the staining reagent to the specimen, and spacing the patch from the surface of the substrate to separate the contact surface from the surface of the substrate from one end to another end of the contact surface, so that the patch is in a third posture in which the patch is oblique to the surface of the substrate.

A device for controlling the posture of a patch according to another aspect of the present disclosure may include a substrate fixing unit for fixing the substrate on which the specimen is placed, a kit including at least one patch receiving member, which receives the patch, such that at least a portion of the contact surface is exposed to the outside, and a control unit to control the position of the patch receiving member with respect to the substrate. The control unit may control the posture of the patch receiving member such that one side of the patch is brought in contact with the specimen, the contact surface is sequentially brought in contact with the specimen from one side to the other of the patch to prevent the formation of air bubbles between the contact surface and the specimen, the staining reagent is delivered to the specimen through the contact surface of the patch, and the contact surface of the patch is sequentially separated from one side to the other side of the patch to uniformly deliver the staining reagent to the area in which the specimen is distributed.

The means for solving the problem of the present disclosure is not limited to the above-described solutions, and the solutions not mentioned are obvious to those of ordinary skill in the technical field to which the present disclosure belongs from the present specification and attached drawings.

### Advantageous Effects of Disclosure

According to the present disclosure, it is easily contain, deliver, and absorb a substance.

According to the present disclosure, a reaction area of a substance may be provided or a certain environment may be provided to a target area.

In addition, according to the present invention, a diagnosis result having sufficient effectiveness can be obtained using a small amount of specimen.

In addition, according to the present disclosure, the amount of solution required for diagnosis can be significantly reduced by properly controlling the delivery and absorption of substance using a patch.

According to the present disclosure, diagnosis can be performed by simultaneously detecting a plurality of targets, and accordingly, customized diagnosis can be performed.

According to the present disclosure, with respect to the entire area of a sample, generation of air bubbles can be prevented and reagents can be delivered evenly.

According to the present disclosure, a sample can be tested while damage to the sample is minimized.

According to the present disclosure, foreign substances and reaction residues can be easily removed from a sample.

The effect of the present disclosure is not limited to the above-described effects, and effects that are not described herein will be clearly understood by those of ordinary skill in the art to which the present disclosure belongs from the present specification and the accompanying drawings.

### BRIEF DESCRIPTION OF DRAWINGS

FIG. 1 illustrates a detailed illustration of an example of a patch according to the present application.

FIG. 2 illustrates a detailed illustration of an example of a patch according to the present application.

FIG. 3 illustrates an illustration of a patch function according to the present application to describe providing of a reaction space as an example of functions of the patch.

FIG. 4 illustrates an illustration of a patch function according to the present application to describe providing of a reaction space as an example of functions of the patch.

FIG. 5 illustrates an illustration of a patch function according to the present application to describe delivering of a substance as an example of functions of the patch.

FIG. 6 illustrates an illustration of a patch function according to the present application to describe delivering of a substance as an example of functions of the patch.

FIG. 7 illustrates an illustration of a patch function according to the present application to describe delivering of a substance as an example of functions of the patch.

FIG. 8 illustrates an illustration of a patch function according to the present application to describe delivering of a substance as an example of functions of the patch.

FIG. 9 illustrates an illustration of a patch function according to the present application to describe delivering of a substance as an example of functions of the patch.

FIG. 10 illustrates an illustration of a patch function according to the present application to describe delivering of a substance as an example of functions of the patch.

FIG. 11 illustrates an illustration of a patch function according to the present application to describe delivering of a substance as an example of functions of the patch.

FIG. 12 illustrates an illustration of a patch function according to the present application to describe delivering of a substance as an example of functions of the patch.

FIG. 13 illustrates an illustration of a patch function according to the present application to describe delivering of a substance as an example of functions of the patch.

FIG. 14 illustrates an illustration of a patch function according to the present application to describe absorption of a substance as an example of functions of the patch.

FIG. 15 illustrates an illustration of a patch function according to the present application to describe absorption of a substance as an example of functions of the patch.

FIG. 16 illustrates an illustration of a patch function according to the present application to describe absorption of a substance as an example of functions of the patch.

FIG. 17 illustrates an illustration of a patch function according to the present application to describe absorption of a substance as an example of functions of the patch.

FIG. 18 illustrates an illustration of a patch function according to the present application to describe absorption of a substance as an example of functions of the patch.

FIG. 19 illustrates an illustration of a patch function according to the present application to describe absorption of a substance as an example of functions of the patch.

FIG. 20 illustrates an illustration of a patch function according to the present application to describe absorption of a substance as an example of functions of the patch.

FIG. 21 illustrates an illustration of a patch function according to the present application to describe absorption of a substance as an example of functions of the patch.

FIG. 22 illustrates an illustration of a patch function according to the present application to describe absorption of a substance as an example of functions of the patch.

FIG. 23 illustrates an illustration of a patch function according to the present application to describe providing of an environment as an example of functions of the patch.

FIG. 24 illustrates an illustration of a patch function according to the present application to describe providing of an environment as an example of functions of the patch.

FIG. 25 illustrates an illustration of a patch function according to the present application to describe providing of an environment as an example of functions of the patch.

FIG. 26 illustrates a diagram illustrating a case in which a patch absorbs or delivers a substance, according to an embodiment of a patch according to the present application.

FIG. 27 illustrates a diagram illustrating a case in which a patch absorbs or delivers a substance, according to an embodiment of a patch according to the present application.

FIG. 28 illustrates a diagram illustrating a case in which a patch absorbs or delivers a substance, according to an embodiment of a patch according to the present application.

FIG. 29 illustrates a diagram illustrating a case in which a patch absorbs or delivers a substance, according to an embodiment of a patch according to the present application.

FIG. 30 illustrates a diagram illustrating a case in which a patch absorbs or delivers a substance, according to an embodiment of a patch according to the present application.

FIG. 31 illustrates a diagram illustrating a case in which a patch absorbs or delivers a substance, or provides an environment, according to an embodiment of a patch according to the present application.

FIG. 32 illustrates a diagram illustrating a case in which a patch absorbs or delivers a substance, or provides an environment, according to an embodiment of a patch according to the present application.

FIG. 33 illustrates an embodiment of a plurality of patches as an example of a patch according to the present application.

FIG. 34 illustrates an embodiment of a plate including a plurality of patches and a plurality of target areas as an example of a patch according to the present application.

FIG. 35 illustrates an example of a substrate and a biological sample according to an embodiment of the present application.

FIG. 36 illustrates a flowchart of a test method according to an embodiment of the present disclosure.

FIG. 37 illustrates an example of improperly staining when blood smeared on a substrate is stained.

FIG. 38 illustrates an example of improperly staining when blood smeared on a substrate is stained.

FIG. 39 illustrates a flowchart of a patch posture control method according to an embodiment of the present disclosure.

FIG. 40 illustrates contacting of a patch with a specimen in a patch posture control method according to an embodiment of the present disclosure.

FIG. 41 illustrates an embodiment in which the contact area between the patch and the specimen is expanded in the order of time.

FIG. 42 illustrates an embodiment in which the contact area between the patch and the specimen is shifted in the order of time.

FIG. 43 illustrates an example of blood unevenly smeared on a substrate.

FIG. 44 illustrates an embodiment in which the contact area between the patch and the specimen is expanded.

FIG. 45 illustrates a side view of a patch, a patch supporting block, and a substrate according to some embodiments of the present disclosure.

FIG. 46 schematically illustrates a process of delivering a reagent to a biological sample by using a test device according to an embodiment of the present disclosure.

FIG. 47 schematically illustrates a process of delivering a reagent to a biological sample by using a test device according to an embodiment of the present disclosure.

FIG. 48 schematically illustrates a process of delivering a reagent to a biological sample by using a test device according to an embodiment of the present disclosure.

FIG. 49 illustrates a flowchart of a test method according to an embodiment of the present disclosure.

FIG. 50 illustrates an embodiment in which the contact area between a patch and a biological sample is reduced.

FIG. 51 illustrates an embodiment in which the contact area between a patch and a biological sample is reduced.

FIG. 52 illustrates a flowchart of a patch posture control method according to an embodiment of the present disclosure.

FIG. 53 illustrates an operation of a test device according to an embodiment of the present disclosure in the order of time.

FIG. 54 illustrates an operation of a test device according to an embodiment of the present disclosure in the order of time.

FIG. 55 illustrates an operation of a test device according to an embodiment of the present disclosure in the order of time.

FIG. 56 illustrates a patch receiving block according to an embodiment of the present disclosure.

FIG. 57 illustrates a specific example of a test device according to an embodiment of the present disclosure.

FIG. 58 illustrates an operation of a test device according to an embodiment of the present disclosure.

FIG. 59 illustrates a patch receiving block according to an embodiment of the present disclosure.

FIG. 60 illustrates a specific example of a test device according to an embodiment of the present disclosure.

FIG. 61 schematically illustrates an embodiment of a test method according to a present disclosure in the order of time.

FIG. 62 schematically illustrates an embodiment of a test method according to a present disclosure in the order of time.

FIG. 63 schematically illustrates an embodiment of a test method according to a present disclosure in the order of time.

FIG. 64 is a flowchart illustrating an embodiment of a test method according to the present disclosure.

FIG. 65 is a flowchart illustrating an embodiment of a test method according to the present disclosure.

FIG. 66 is a flowchart illustrating an embodiment of a test method according to the present disclosure.

FIG. 67 is a flowchart illustrating an embodiment of a test method according to the present disclosure.

FIG. 68 illustrates an example of a frame according to an embodiment of the present disclosure.

FIG. 69 illustrates an example of a patch receiving member according to an embodiment of the present disclosure.

FIG. 70 illustrates a view of a base according to an embodiment of the present disclosure.

FIG. 71 illustrates a view of a kit according to an embodiment of the present disclosure.

FIG. 72 illustrates an operation of a test device according to an embodiment of the present disclosure in the order of time.

FIG. 73 illustrates an operation of a test device according to an embodiment of the present disclosure in the order of time.

FIG. 74 illustrates an operation of a test device according to an embodiment of the present disclosure in the order of time.

FIG. 75 illustrates a view of a test device according to an embodiment of the present disclosure.

FIG. 76 illustrates an operation of a test device according to an embodiment of the present disclosure in the order of time.

FIG. 77 illustrates an operation of a test device according to an embodiment of the present disclosure in the order of time.

FIG. 78 illustrates a specific example of a test device according to an embodiment of the present disclosure.

FIG. 79 illustrates an example of a medium receiving member according to an embodiment of the present disclosure.

FIG. 80 illustrates a view of a storage medium according to an embodiment of the present disclosure.

FIG. 81 schematically illustrates a test method according to an embodiment of the present disclosure in the order of time.

FIG. 82 schematically illustrates a test method according to an embodiment of the present disclosure in the order of time.

### MODE OF DISCLOSURE

The objectives of the embodiments described in this specification are to clearly explain the concept of present disclosure to those with ordinary knowledge in the art to which the present disclosure belongs, the present disclosure is not limited to the embodiments described herein, and the scope of the present disclosure should be construed as including modifications or variations thereof that do not depart from the spirit of the present disclosure.

The terms used in this specification have been selected as a general term that is currently widely used in consideration of the function in the present disclosure, but they varies depending on the intention, custom, or the emergence of new technology in the technical field to which the present disclosure belongs. However, when a specific term is defined and used in an arbitrary meaning, the meaning of the term will be separately described. Therefore, terms used in the present specification should be interpreted based on the actual meaning thereof and the context thereof described throughout the present specification, rather than a simple name of the terms.

The drawings attached to the present specification are provided for easily explaining the present disclosure, and since the configurations illustrated in the drawings may be exaggerated as necessary to help understanding of the present disclosure, the present disclosure is not limited by the drawings.

In the present specification, when it is determined that a detailed description of a known configuration or function related to the present disclosure may obscure the subject matter of the present disclosure, a posture description thereof will be omitted as necessary.

### 1. Patch

### 1.1 Definition of Patch

The present application discloses a patch for managing a liquid substance.

The liquid substance is a substance that is flowable and may refer to a substance that may exist in a liquid state.

The liquid substance may be a single- component substance having liquidity.

In one or more embodiments, the liquid substance may be a mixture including a multi-components substance.

When the liquid substance is a single-component substance, the liquid substance may be a substance consisting of a single element or a compound containing a plurality of chemical elements.

When the liquid substance is a mixture, some of the components may function as a solvent and the others may function as a solute. That is, the mixture may be a solution.

Meanwhile, the multi-components substance constituting the mixture may be uniformly distributed. In one or more embodiments, the mixture including the multi-components substance may be a uniformly mixed mixture.

The multi-components substance may include a solvent and a substance that is not soluble in the solvent and is uniformly distributed.

Meanwhile, some of the multi-components material may be non-uniformly distributed. The non-uniformly distributed substance may include a particle component that is non-uniformly distributed in the solvent. In this case, the non-uniformly distributed particle component may be a solid phase.

For example, the substance that can be managed by using the patch may be 1) a single-component liquid, 2) a solution, or 3) a colloid phase, or in some cases, 4) solid particles that are non-uniformly distributed in different liquid substances.

Hereinafter, the patch according to the present application will be described in detail.

### 1.2 General Characteristics of Patch

### 1.2.1 Composition

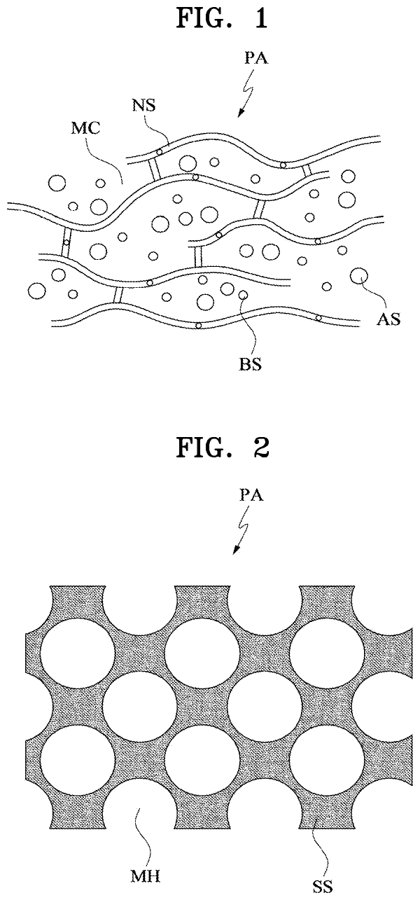

FIGS. 1 and 2 are diagrams each showing an example of a patch according to the present application.

Hereinafter, a patch PA according to the present application will be described with reference to FIGS. 1 and 2.

Referring to FIG. 1, the patch PA according to the present application may include a net structure NS and a liquid substance.

Here, the liquid substance may include a base substance BS and an additive substance AS.

In addition, the patch PA may be of a gel type. The patch PA may be implemented as a gel-like structure in which colloid molecules are bound to form a net structure.

The patch PA according to the present application may include a three-dimensional net structure NS as a structure for managing the liquid substance SB. The net structure NS may be a continuously distributed solid structure. The net structure NS may have a network structure in which a plurality of fine threads are entangled. However, the net structure NS is not limited to the form of a network in which a plurality of fine threads are entangled, and may be implemented in the form of an arbitrary three-dimensional matrix formed by connecting a plurality of fine structures. For example, the net structure NS may be a skeleton structure including a plurality of micro-cavities. In other words, the net structure NS may form a plurality of micro-cavities MC.

FIG. 2 illustrates the structure of a patch structure according to an embodiment of the present application. Referring to FIG. 2, the net structure of the patch PA may have a sponge structure SS. In this case, the net structure of the sponge structure SS may include a plurality of fine holes MH. Hereinafter, the fine holes and the micro-cavities MC may be used interchangeably with each other, and unless otherwise noted, the micro-cavities MC is defined as including the concept of fine holes MH.

In addition, the net structure NS may have a regular or irregular pattern. Furthermore, the net structure NS may include both an area having a regular pattern and an area having an irregular pattern.

The density of the net structure NS may have a certain range of value. In one embodiment, the certain range may be determined within such a range that the shape of the liquid substance SB captured in the patch PA is maintained in a shape corresponding to the patch PA. The density may be defined as the degree of compactness of the net structure NS, or the mass ratio and volume ratio at which the net structure NS occupies the patch.

The patch according to the present application may manage the liquid substance SB by having a three-dimensional net structure.

The patch PA according to the present application may include the liquid substance SB, and the fluidity of the liquid substance SB included in the patch PA may be limited by the shape of the net structure NS of the patch PA.

The liquid substance SB may freely flow within the net structure NS. In other words, the liquid substance SB is located in a plurality of micro-cavities formed by the net structure NS. The exchange of liquid substance SBs may occur between adjacent micro-cavities. In this case, the liquid substance SB may exist in such a form that the liquid substance SB penetrates into the frame structure that forms the net structure. In this case, nano-sized pores, through which the liquid substance SB may penetrate, may be formed in the frame structure.

Further, depending on the molecular weight of the liquid substance SB captured in the patch PA or the size of the particles thereof, whether or not the liquid substance SB is injected into the frame structure of the net structure may be determined. A substance having a relatively high molecular weight may be captured in the micro-cavities, and a substance having a relatively small molecular weight may be introduced into the micro-cavities and/or the frame structure of the net structure NS and captured.

The term ‘capture’ as used herein may be defined as meaning a state in which the liquid substance SB is located in the plurality of micro-cavities and/or the nano-sized pores formed by the net structure NS. In addition, the state in which the liquid substance SB is captured in the patch PA is defined as including a state in which the liquid substance SB may flow between the micro-cavities and/or the nano-sized pores, as described above.

The liquid substance SB includes the base substance BS and the additive substance AS.

The base substance BS may be the liquid substance SB having fluidity.

The additive substance AS may be a substance having fluidity by being mixed with the base substance BS. In other words, the base substance BS may be a solvent. The additive substance AS may be a solute that is soluble in the solvent or a particle that is insoluble in the solvent.

The base substance BS may be a substance that may flow inside the matrix formed by the net structure NS. In an embodiment, the base substance BS may be uniformly distributed in the net structure NS, and may be distributed only in an area of the net structure NS. The base substance BS may be a liquid consisting of a single component.

The additive substance AS may be a substance that is mixed with the base substance BS or soluble in the base substance BS. For example, the additive substance AS may function as a solute and the base substance BS as a solvent. The additive substance AS may be uniformly distributed in the base substance BS.

The additive substance AS may be fine particles that do not dissolve in the base substance BS. For example, the additive substance AS may contain microparticles such as colloidal molecules and microorganisms.

The additive substance AS may contain particles larger than the micro-cavities formed by the net structure NS. When the size of the micro-cavities is smaller than the size of the particles contained in the additive substance AS, the fluidity of the additive substance AS may be limited.

In addition, according to an embodiment, the additive substance AS may contain a component selectively included in the patch PA.

In one or more embodiments, the additive substance AS does not necessarily mean a substance that is inferior or superior in quantity or functionally in relation to the base substance BS.

In the following, the characteristics of the liquid substance SB captured in the patch PA may be regarded as the properties of the patch PA. That is, the characteristics of the patch PA may depend on the properties of the substance captured in the patch PA.

### 1.2.2 Characteristics

The patch PA according to the present application may include the net structure NS as described above. The patch PA may manage the liquid substance SB by using the net structure NS. The patch PA may allow the liquid substance SB captured in the patch PA to retain at least some of intrinsic properties thereof.

For example, in the area of the patch PA in which the liquid substance SB is distributed, the diffusion of a substance may occur or a force, such as a surface tension, may affect.

The patch PA may provide a liquid environment in which a target material is diffused by a difference in the thermal motion, density or concentration of the substance. In general, the term ‘diffusion’ means that particles constituting a substance spread from a higher concentration to a lower concentration due to the difference in concentration. This diffusion phenomenon may be basically understood as a resultant phenomenon caused by the motion of molecules (translational motion in a gas or liquid, vibrational motion in a solid, etc.). In the present application, the term ‘diffusion’ refers to a phenomenon in which particles spread from a high concentration to a low concentration due to a difference in concentration or density, and may also refer to the phenomenon in which particles move due to irregular motion of molecules occurring even when concentrations thereof are non-uniform. In addition, the expression ‘irregular motion’ of particles is also used in the same meaning as ‘diffusion’ unless defined otherwise.

The target material to be diffused may be a solute dissolved in the liquid substance SB, and the solute may be provided in a solid, liquid or gaseous state.

In an embodiment, the non-uniformly distributed substance of the liquid substance SB captured by the patch PA may diffuse in the space provided by the patch PA. In other words, the additive substance AS may diffuse in the space defined by the patch PA.

The non-uniformly distributed substance of the liquid substance SB that the patch PA manages or the additive substance AS may diffuse within the micro-cavities provided by the net structure NS of the patch PA. In addition, the area, in which the non-uniformly distributed substance or the additive substance AS may diffuse, may be changed when the patch PA contacts or is connected to other substances.

In addition, even after the concentration of the non-uniformly distributed substance or the additive substance AS becomes uniform due to the diffusion of the non-uniformly distributed substance or additive substance AS in the patch PA or in the external area connected to the patch PA, the substance or the additive substance AS may be constantly moved by the irregular motion of molecules within the inside of the patch PA and/or in the external area connected to the patch PA.

The patch PA may be implemented to have hydrophilic or hydrophobic properties. In other words, the net structure NS of the patch PA may have hydrophilic or hydrophobic properties.

When the properties of the net structure NS are similar to the properties of the liquid substance SB, the net structure NS may more effectively manage the liquid substance SB.

The property of the base substance BS may be a hydrophilic substance having a polarity or a hydrophobic substance having no polarity. In addition, the property of the additive substance AS may be hydrophilic or hydrophobic.

The properties of the liquid substance SB may be associated with the substance BS and/or the additive substance AS. For example, when both the base substance BS and the additive substance AS may be hydrophilic, the liquid substance SB may be hydrophilic, and when both the base substance BS and the additive substance AS are hydrophobic, the liquid substance SB may be hydrophobic. When the polarity of the base substance BS is different from the additive substance AS, the liquid substance SB may be hydrophilic or hydrophobic.

When the polarity of the net structure NS and the polarity of the liquid substance SB are both hydrophilic or hydrophobic, an attractive force may act between the net structure NS and the liquid substance SB. When the polarity of the net structure NS and the polarity of the liquid substance SB are opposite to each other, for example, when the polarity of the net structure NS is hydrophobic and the liquid substance SB is hydrophilic, a repulsive force may act between the net structure NS and the liquid substance SB.

Based on the above-described properties, the patch PA may be used alone, in plural, or in combination with other media to induce a target reaction. Hereinafter, a functional aspect of the patch PA will be described.

However, for convenience of description, it is assumed that the patch PA is a gel phase that is capable of containing a hydrophilic solution. In other words, the following description will be made based on the assumption that the net structure NS of the patch PA has hydrophilic properties, unless defined otherwise.

However, the scope of the present application should not be interpreted as being limited to the gel-phase patch PA having hydrophilic properties. That is, the scope of the present application may extend to, in addition to the gel-phase patch PA containing a solution having hydrophobic properties, a gel-phase patch PA, from which the solvent has been removed, and a sol-phase patch PA that implements the functions according to the present application.

### 2. Functions of Patch

A patch according to the present application may have several useful functions due to the above-described characteristics. In other words, the patch may be involved in the behavior of the liquid substance SB by occupying the liquid substance SB.

Accordingly, hereinafter, according to the behavior of the substance in relation to the patch PA, descriptions will be made in the aspects of a reservoir function in which the state of the substance is defined in a certain area formed by the patch PA and a channeling function in which the state of the substance is defined in the certain area and ( [symbol] ) the external area.

### 2.1 Reservoir

### 2.1.1 Significance

The patch PA according to the present application may capture the liquid substance SB as described above. In other words, the patch PA may perform the function of a reservoir.

The patch PA may capture the liquid substance SB in a plurality of micro-cavities formed in the net structure NS through the net structure NS. The liquid substance SB may occupy at least some of the micro-cavities formed by the three-dimensional net structure NS of the patch PA, or may penetrate into nano-sized pores formed in the net structure NS.

The liquid substance SB located in the patch PA does not lose the liquid properties thereof even when distributed in the micro-cavities. That is, the liquid substance SB has liquidity even in the patch PA, and, in the liquid substance SB distributed in the patch PA, diffusion of the substance may occur, and a suitable solute may be dissolved in the substance.

Hereinafter, the reservoir function of the patch PA will be described in detail.

### 2.1.2 Containing

According to the present application, the patch PA may capture a target material due to the properties as described above. The patch PA may have a certain degree of resistance to changes in the external environment. Through this, the patch PA may maintain the substance being captured. The liquid substance SB, which is the target to be captured, may occupy the three-dimensional net structure NS.

Hereinafter, the function of the patch PA as described above will be referred to as containing, for convenience.

However, the wording ‘the patch PA contains the liquid substance’ refers to that the liquid substance is contained in a space formed by the net structure and/or that the liquid substance is contained in a frame structure constituting the net structure NS.

The patch PA may contain the liquid substance SB. For example, due to the attractive force acting between the net structure NS of the patch PA and the liquid substance SB, the patch PA may contain the liquid substance SB. The liquid substance SB may be contained while being bound to the net structure NS with at least a certain strength of an attractive force.

The properties of the liquid substance SB contained in the patch PA may be classified according to the properties of the patch PA. In an embodiment, when the patch PA has a hydrophilic property, the patch PA may be bound to a hydrophilic liquid substance SB having a polarity so that the hydrophilic liquid substance SB is contained in the three-dimensional micro-cavities. In an embodiment, when the patch PA has a hydrophobic property, a hydrophobic liquid substance SB may be contained in the micro-cavities of the three-dimensional net structure NS.

Further, the amount of the substance contained in the patch PA may be proportional at a certain ratio to the volume of the patch PA. In other words, the amount of substance contained in the patch PA may be proportional to the amount of the three-dimensional net structure NS as a support contributing to the shape of the patch PA. However, the relationship between the amount of the substance contained and the volume of the patch PA does not have a certain proportionality constant, and the relationship between the amount of the substance to be able to be contained and the volume of the patch PA may vary depending on the design or manufacturing method of the net structure.

The amount of the substance contained in the patch PA may be decreased due to evaporation, dropping, etc. over time. In addition, by adding a substance to the patch PA, the amount of the substance contained in the patch PA may be increased or maintained. For example, a moisture preservative for suppressing evaporation of moisture may be added to the patch PA.

The patch PA may be implemented in a form that is easy to contain the liquid substance SB. That is, the patch PA may be implemented in order to minimize the degeneration of the substance when the substance is affected by the environment such as humidity, light quantity, temperature, etc. For example, in order to prevent the patch PA from being denatured by external factors such as bacteria, the patch PA may be treated with a bacteria inhibitor or the like.

Meanwhile, the liquid substance SB having a plurality of components may be contained in the patch PA. At this time, the multi-components substance may be placed together in the patch PA before the reference time point, or a first substance injected into the patch PA is first contained in the patch PA, and then, a secondary substance may be contained in the patch PA after a certain period of time. In an embodiment, when two components of liquid substance SB are contained in the patch PA, two components may be contained in the patch PA during the manufacture of the patch PA. In an embodiment, only one component may be contained in the patch PA and then the other one may be contained later, during the manufacture of the patch PA. In an embodiment, the two components may be sequentially contained after the fabrication of the patch PA.

In addition, the substance contained in the patch PA may basically exhibit fluidity, as described above, and may undergo irregular or diffuse motion due to molecular motion in the patch PA.

### 2.1.3 Providing of Reaction Space

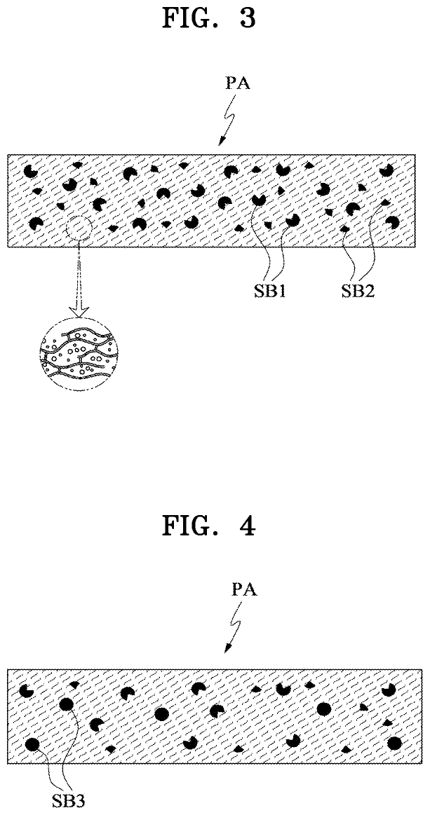

FIGS. 3 and 4 each show an illustration of a patch function according to the present application to describe providing of a reaction space as an example of functions of the patch.

As illustrated in FIGS. 3 and 4, the patch PA according to the present application may provide a space. In other words, the patch PA may provide a space for the liquid substance SB to move, by using the space formed by the net structure NS and/or the space constituting the net structure NS.

The patch PA may provide a space for activities other than diffusion of particles and/or irregular motion of particles (hereinafter referred to as activities other than diffusion). Activities other than diffusion may refer to a chemical reaction, but are not limited thereto. For example, the activities may refer to a change in the physical state. In an embodiment, activities other than diffusion are a chemical reaction in which the chemical composition of the substance changes before and after the activity, a specific binding reaction between components contained in the substance, homogenization of a solute or particles contained in the substance and non-uniformly distributed, or aggregation of some components contained in the substance, or biological activity of some of the substance.

Meanwhile, when a plurality of substances are involved in the activity, the substances may be o-located in the patch PA before the reference time point. The substances may be sequentially injected.

By changing the environmental conditions of the patch PA, the efficiency of the function of providing space for activities other than the diffusion of the patch PA may be improved. For example, by changing the temperature conditions of the patch PA or adding electrical conditions, the activity may be promoted or the initiation of the activity may be induced.

Referring to FIGS. 3 and 4, a first substance SB1 and a second substance SB2 located in the patch PA may react each other inside the patch PA to transform into a third substance SB3, or may generate the third substance SB3.

### 2.2 Channel

### 2.2.1 Significance

Transfer of a substance may occur between the patch PA and the external area. In an embodiment, a substance may be transferred from the patch PA to an external area of the patch PA, or from the external area to the patch PA.

The patch PA may form a path of movement of a substance or may participate in the movement of a substance. In an embodiment, the patch PA may be involved in the movement of the liquid substance SB captured in the patch PA or the movement of the foreign substance through the liquid substance SB captured in the patch PA. The base substance BS or the additive substance AS may escape from the patch PA, or a foreign substance may be introduced into the patch PA from an external area.

The patch PA may function as a passage for the movement of a substance. That is, the patch PA may participate in the movement of a substance and may function as a channel for the movement of a substance. The patch PA may provide a channel for the movement of a substance due to the inherent properties of the liquid substance SB.

The patch PA may have a state in which the liquid substance SB can move between the patch PA and the external area or a state in which the liquid substance SB cannot move between the patch PA and the external area, according to whether the patch PA is connected to the external area or not. In addition, when channeling between the patch PA and the external area starts, the patch PA may have unique functions.

Hereinafter, the state in which the substance can move and the state in which the substance cannot move will be first described, and the performing of the specific functions of the patch PA will be described in detail in connection with whether the patch PA and the external area are connected.

Basically, the basic reason for the movement of the liquid substance SB between the patch PA and the external area is due to the irregular motion and/or diffusion of the substance. However, in order to control the movement of substances between the patch PA and the external area, external environmental factors (for example, control of temperature conditions, and control of electrical conditions) may be controllable, which has been already described above.

### 2.2.2 Movable State

When the substance is in a movable state, fluidity may occur between the liquid substance SB captured in the patch PA and/or the substance located in the external area. When the substance is in a movable state, a movement of the substance captured in the patch PA may occur between the liquid substance SB and the external area.

For example, when the substance is in a movable state, the liquid substance SB or some components of the liquid substance SB may diffuse into the external area or move thereto due to irregular motion. In an embodiment, when the substance is in a movable state, a foreign substance located in the external area or some components of the foreign substance may diffuse into the liquid substance SB of the patch PA or move thereto by irregular motion.

The substance in a movable state may be triggered through contact. The wording ‘contact’ refers to a connection between that the liquid substance SB captured by the patch PA and the external area. The wording ‘contact’ refers to overlapping of the flow area of the liquid substance SB and at least a portion of the external area. The wording ‘contact’ refers to a connection between the foreign substance and at least a portion of the patch PA. The wording ‘the substance is in a movable state’ may be understood that the range, in which the captured liquid substance SB flows, is expanded. In other words, when the substance is in a movable state, the flowable range of the substance in the liquid phase may be expanded to an extent that the flowable range includes at least a portion of the external area of the captured liquid substance SB. For example, when the liquid substance SB is in contact with the external area, the range in which the captured liquid substance SB flows may be expanded to an extent that the flowable range includes at least a portion of the contacted external area. In an embodiment, when the external area is an external plate, the area in which the liquid substance SB flows may be expanded to an extent that the flowable range includes an area in contact with the liquid substance SB of the external plate.

2.2.3 Immovable State

When the substance is in an immovable state, a movement of the substance captured in the patch PA may not occur between the liquid substance SB and the external area. However, each of the liquid substance SB captured in the patch PA and the foreign substance located in the external area may move separately.

When the substance is in an immovable state, the contact may not occur. In other words, when the patch PA does not contact the external area, the movement of a substance may not occur between the liquid substance SB remaining in the patch PA and the external area or in the foreign substance.

In an embodiment, the wording “the contact does not occur” refers to the state in which the liquid substance SB captured by the patch PA is not connected to the external area. In an embodiment, the wording ‘the contact does not occur’ refers to the state in which the liquid substance SB is not connected to the external area located in the external area. For example, the state in which the substance is in the immovable state may be caused by the separation of the patch PA from the external area.

The terms ‘movable state’ and ‘immovable state’ are distinguishable from each other. However, transitions between states may occur due to, for example, the passage of time or changes in the environment. In an embodiment, the patch PA may be in a movable state and then in an immovable state. In an embodiment, the patch PA may be in an immovable state and then in a movable state. In an embodiment, the patch PA is in a movable state, and then, in an immovable state, and then, in a movable state.

### 2.2.4 Classification of Functions

2.2.4.1 Delivering

According to the present application, due to the characteristics described above, at least a portion of the liquid substance SB occupied by the patch PA may be delivered to a target external area. The delivery of the substance may refer to a case in which, when a certain condition is satisfied, a portion of the liquid substance SB captured by the patch PA is separated from the patch PA.

The wording ‘the separation of a portion of the liquid substance SB’ refers to a case in which the portion thereof is extracted, emitted, or released from an area affected by the patch PA. Such a separation may be understood as a smaller concept of the channel function of the patch PA, and as defined as the delivery of a substance located in the patch PA to the outside the patch PA.

The target external area may be another patch PA, a dry area, or a liquid area.

The certain condition for causing the delivery may be environmental conditions including temperature change, pressure change, electrical characteristic change, and physical state change. For example, when the patch PA contacts an object having a stronger binding force with the liquid substance SB than the net structure NS of the patch PA, the liquid substance SB may chemically bind to the object, and as a result, at least a portion of the liquid substance may be delivered to the object.

Hereinafter, the function of the patch PA as described above will be referred to as the delivery, for convenience.

The delivery may occur when the liquid substance SB undergoes via/through a movable state between the patch PA and the external area and an immovable state between the patch PA and the external area.

In an embodiment, when the liquid substance SB is in the in a movable state, the liquid substance SB may diffuse between the patch PA and the external area or may move to the external area by irregular motion. In other words, the base solution and/or additive substance AS contained in the liquid substance SB may move from the patch PA to the external area. When the liquid substance SB is in the immovable state, the movement thereof between the patch PA and the external area may not occur. In other words, some of the substance that have been moved from the patch PA to the external area due to the diffusion and/or irregular motion of the liquid substance SB may not move back to the patch PA due to the transition from the movable state to the immovable state. Thus, some of the liquid substance SB may be delivered to the external area.

The deliver may be performed according to a difference between the attractive force between the liquid substance SB and the net structure NS and the attractive force between the liquid substance SB and the external area or the foreign substance. The attraction may result from polarity similarity or specific binding relationships.

In an embodiment, when the liquid substance SB is hydrophilic and the external area or the foreign substance is more hydrophilic than the net structure NS of the patch PA, at least a portion of the liquid substance SB that has been captured by the patch PA may be delivered to the external area through the movable state and the immovable state.

The delivery of the liquid substance SB may be selectively performed. For example, when there is a specific binding relationship between some components contained in the liquid substance SB and the foreign substance, the some components may be selectively delivered through the movable state and the immovable state.

In an embodiment, assuming that the patch PA delivers a substance to an external plate (PL) in the form of a flat plate, a substance that specifically binds to some of the liquid substance SB captured in the patch PA (for example, part of the solute) may be applied on the external plate PL. At this time, the patch PA selectively delivers, to the plate PL from the patch PA, a part of the solute specifically binding to the substance applied to the external plate PL, through the movable state and the non-movable state.

Hereinafter, according to some examples of other areas to which the substance is moved, the delivery function of the patch PA will be described. However, in the detailed description, the concepts ‘release’ of the liquid substance SB and ‘deliver’ of the liquid substance SB may be interchangeable.

Herein, a case in which the liquid substance SB is delivered from the patch

PA to a separate external plate PL will be described. For example, it may be considered a case in which a substance is moved from the patch PA to the plate PL such as a slide glass.

As the patch PA and the plate PL come into contact each other, at least a portion of the liquid substance SB captured in the patch PA may move by diffusion or an irregular motion. When the patch PA is separated from the plate PL, a portion of the substance (that is, some of the liquid substance SB) that has been moved from the patch PA to the plate PL may not move back to the patch PA.

As a result, the portion of the substance may be delivered from the patch PA to the plate PL. At this time, the portion of the substance to be delivered may be the additive substance AS. In order for the substance in the patch PA to be ‘delivered’ due to the contact and the separation, an attractive force and/or a bonding force acting between the substance and the plate PL is needed, and the attractive force and/or a bonding force may be greater than the attractive force acting between the substance and the patch PA. Therefore, when the ‘delivery condition’ is not satisfied, the delivery of a substance between the patch PA and the plate PL may not occur.

In addition, the delivery of the substance may be controllable by providing a temperature or electrical condition to the patch PA.

The movement of a substance from the patch PA to the plate PL may depend on the contact area between the patch PA and the plate PL. For example, the efficiency of the movement of a substance between the patch PA and the plate PL may vary depending on the contact area of the patch PA and the plate PL.

When the patch PA includes a plurality of components, only some components may be selectively moved to the plate PL. In an embodiment, a substance specifically binding to some of the components may be fixed on the plate PL. At this time, the substance fixed on the plate PL may be in a liquid or solid state, and may be fixed in the separate area. In this case, due to contact between the patch PA and the separate area, some substances of the components move to the plate PL to form a specific binding, and when the patch PA is separated from the plate PL, only some components may be selectively released to the plate PL.

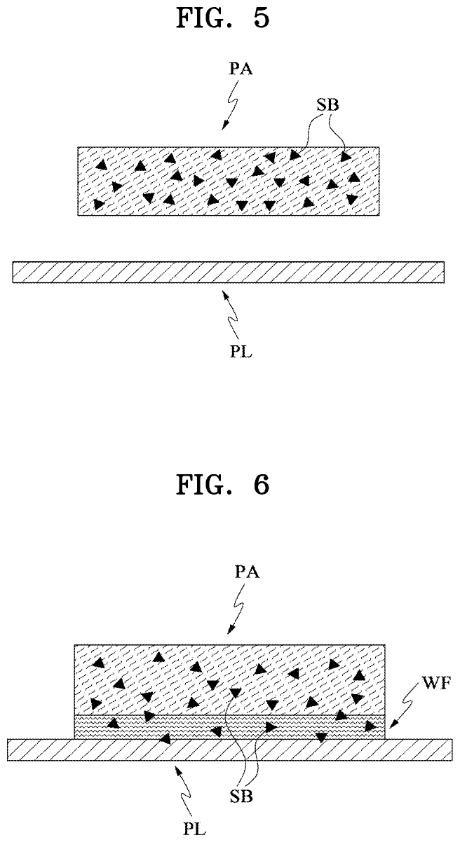

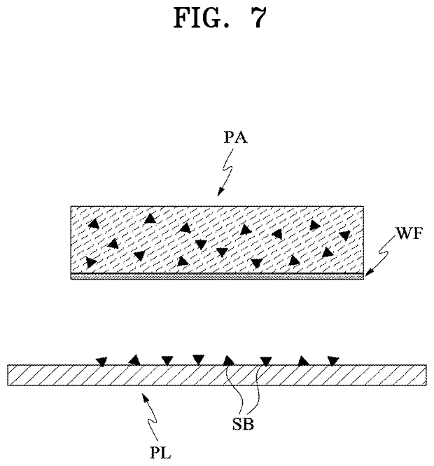

FIGS. 5 to 7 illustrate an example of the delivery of a substance from the patch PA to the external plate PL, as an example of functions of the patch PA according to the present application. Referring to FIGS. 5 to 7, the patch PA may deliver some of the substance contained in the patch PA to the plate PL by being brought into contact with the plate PL. In this regard, the delivery of the substance may be implemented by bringing the patch and the plate PL into contact each other. In this regard, a water film WF may be formed near a contact surface where the plate and the patch PA contact, and the substance may move through the formed water film WF.

Herein, a case where the liquid substance SB is delivered from the patch PA to a fluidity-having substance SL having fluidity will be described. Here, the fluidity-having substance SL may be a liquid substance that is contained in a separate container or that flows.

As the patch PA and the fluidity-having substance come into contact (for example, the patch PA is put into the solution), at least a portion of the liquid substance SB captured in the patch PA may move to the fluidity-having substance SL by diffusion or an irregular motion. When the patch PA is separated from the fluidity-having substance, some of the liquid substance SB, which has been moved from the patch PA to the fluid substance, may not move back to the patch PA, so that some of the substance that has been in the patch PA may be delivered to the fluidity-having substance.

The movement of a substance between the patch PA and the fluidity-having substance SL may depend on the contact area of the patch PA and the fluidity-having substance SL. For example, depending on the area in which the patch PA and the fluidity-having substance SL contact (for example, the depth at which the patch PA is injected into a solution, etc.), the efficiency of the movement of a substance between the patch PA and the fluidity-having substance SL may vary.

The movement of a substance between the patch PA and the fluidity-having substance SL may depend on the physical separation of the patch PA and the fluidity-having substance SL.

Since the distribution concentration of the additive substance AS in the liquid substance SB may be different from the distribution concentration of the additive substance AS in the fluidity-having substance, the additive substance AS may be delivered from the patch PA to the fluidity-having substance.

However, when the patch PA delivers the liquid substance SB to the fluidity-having substance SL, physical separation between the patch PA and the fluidity-having substance SL is not essential. For example, when the driving force/causal force from the patch PA to the liquid having the fluidity is decreased to a reference value or lower, or disappeared, the movement of a substance may be stopped.

In the ‘delivery’ between the patch PA and the fluidity-having substance SL, the ‘delivery condition’ between the patch PA and the fluidity-having substance SL described above may not be required. That is, substances that have already moved to the fluidity-having substance SL may move in the fluidity-having substance SL by diffusion and/or an irregular motion, and, when the distance between the moved substance and the patch PA is increased to a certain range or more by the movement, it is considered that the substance has been delivered to the fluidity-having substance SL. In the case of the plate PL, the movable range extended by the contact is a very limited range, so the attractive force between the substances moved to the plate PL and the patch PA may act significantly. However, in the relationship of the fluidity-having substance SL and the patch PA, the movable range extend by the contact between the patch PA and the plate PL is relatively wide, so the attractive force between the substances moved to the fluidity-having substance SL and the patch PA may be insignificant.

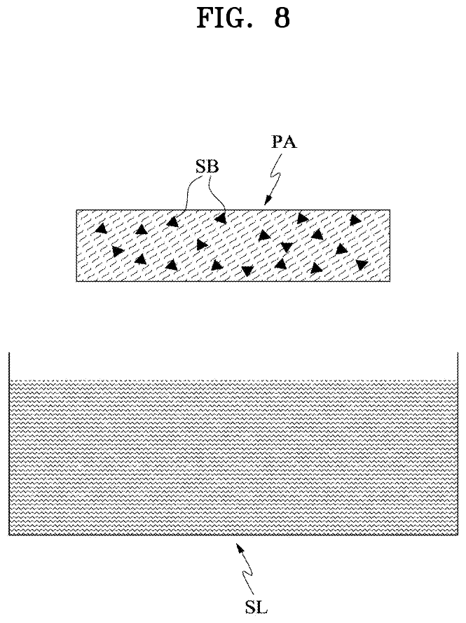

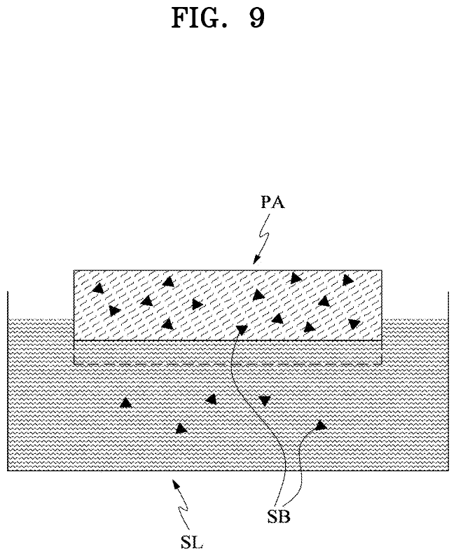

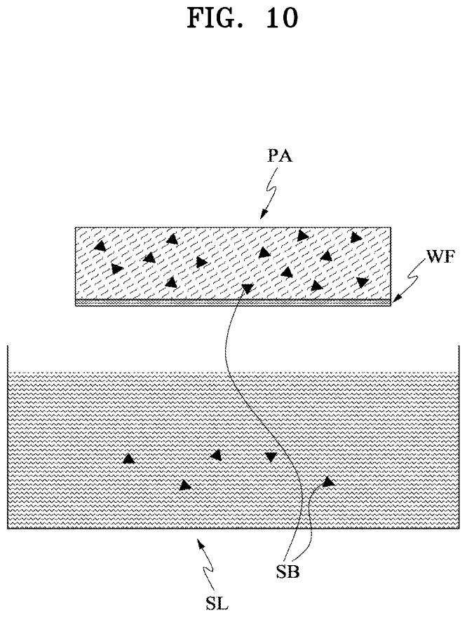

FIGS. 8 to 10 illustrate an example of the delivery of a substance from the patch PA to the fluidity-having substance, as an example of functions of the patch PA according to the present application. Referring to FIGS. 8 to 10, the patch PA may deliver a portion of the substance contained in the patch PA to an external fluidity-having substance. The delivering of the contained portion of the substance may be performed as follows: the patch PA is injected into or in contact with the fluidity-having substance so that a substance can move from the liquid substance SB captured in the patch PA to the fluidity-having substance.

Herein, it is assumed that the substance moves from the patch PA to another patch PA. In an area where the patch PA and the other patch PA contact, at least a portion of the liquid substance SB provided to the patch PA may move to the other patch PA.

In the contact area, the liquid substance SB provided to the patch PA may move, by diffusion, to the other patch PA and vice versa. In this regard, due to the movement of the substance, the concentration of the liquid substance SB provided in each of the patch PAs may be changed. Also in this embodiment, as described above, the patch PA may be separated from the other patch PA, and in this case, some of the liquid substance SB of the patch PA may be delivered to the other patch PA.

The movement of a substance between the patch PA and the other patch PA may be carried out by changes in environmental conditions including changes in physical state.

The movement of a substance between the patch PA and the other patch PA may depend on the contact area of the patch PA and the other patch PA. For example, the efficiency of the movement of a substance between the patch PA and the other patch PA may vary depending on the contact area of the patch PA and the other patch PA.

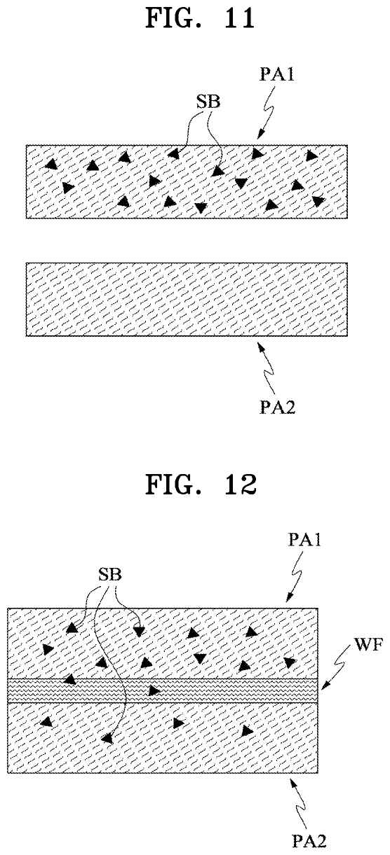

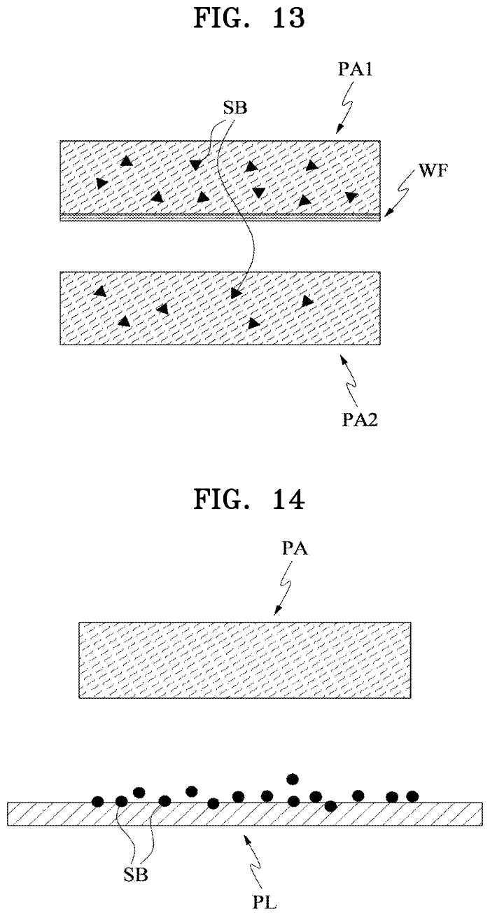

FIGS. 11 to 13 illustrate an example of the delivery of a substance from a patch PA1 to another patch PA2, as an example of functions of the patch PA according to the present application. Referring to FIGS. 11 to 13, the patch PA1 may deliver a portion of the substance contained in the patch PA1 to the other patch PA2.

The delivering of the portion of the substance may be performed in such a condition that the patch PA1 comes into contact with the other patch PA2, and thus, a liquid substance SB captured in the patch PA1 is exchangeable with a substance captured in the other patch PA2.

2.2.4.2 Absorption

The term ‘absorb’, one of the functions of the patch PA according to the present application may be managed in a similar manner as used to describe ‘delivery’ in some embodiments. For example, in the case in which a substance moves due to a difference in concentration of a substance, the movement direction of the moving substance may be controlled by changing the concentration of the liquid substance SB, for example, the concentration of the additive substance AS. In this aspect, the ‘absorb’ and the ‘delivery’ are common. In addition, it would be clearly understood by one of ordinary skill in the art that, even in the aspect of the movement control of a substance through separation of the physical contact and selective absorption of the patch PA, the ‘absorb’ and the ‘delivery’ are common.

The patch PA according to the present application may capture a foreign substance according to characteristics described above. The patch PA may pull a foreign substance existing outside the area defined by the patch PA into an area affected by the patch PA. The pulled foreign substance may be captured together with the liquid substance SB of the patch PA. The pulling of the foreign substance may result from the attraction between the liquid substance SB that has already been captured in the patch PA and the foreign substance. In an embodiment, the pulling of the foreign substance may result from the attraction between the foreign substance and an area of the net structure NS that is not occupied by the liquid substance SB. The pulling of the foreign substance may result from the force of the surface tension.

Hereinafter, the function of the patch PA as described above will be referred to as the absorption, for convenience. The absorption may be understood as a smaller concept of the channel function of the patch PA, and as defined as the movement of a substance located in the patch PA to the outside the patch PA.

The absorption may occur when the patch PA undergoes via/through an movable state of the substance and an immovable state of the substance.

The substance that the patch PA is able to absorb may be in a liquid or solid state. For example, when the patch PA is in contact with a foreign substance containing a solid substance, the absorption of the substance is caused by the attraction between the liquid substance SB located in the patch PA and the solid substance contained in the foreign substance. In an embodiment, when the patch PA contacts a liquid foreign substance, the absorption may be performed by combining the liquid substance SB located in the patch PA and a liquid foreign substance.

The foreign substance absorbed by the patch PA may move into the interior of the patch PA through micro-cavities of the net structure NS constituting the patch PA, or may be distributed on the surface of the patch PA. The distribution position of the foreign substance may be determined depending on the molecular weight of the foreign substance or the size of particles thereof.

During the absorbing, the shape of the patch PA may be deformed. For example, the volume, color, etc. of the patch PA may change. On the other hand, while the patch PA absorbs, the absorbing environment of the patch PA may be activated or delayed by adding external conditions such as temperature change and physical state change.

Hereinafter, in the case in which absorbing occurs, according to several examples of an external area providing a substance absorbed by the patch PA, the absorption, as the function of the patch PA, will be described.

Hereinafter, the case in which the patch PA absorbs a foreign substance from a separate external plate PL will be described. In this regard, the separate external substrate may be, for example, a plate PL that does not absorb the foreign substance, but may enable the foreign substance to be placed thereon.

A substance may be applied on the outer plate PL. For example, a substance may be applied in powder form on the plate PL. The substance applied on the plate PL may be a single component or a mixture of multiple components.

The plate PL may have a flat-plate shape. In addition, the plate PL may be modified in shape to improve the storage properties of the substance. For example, a well may be formed in the plate PL to improve storage performance, or the surface of the plate PL may be modified or patterned to have an uneven structure to improve the contact property thereof with the patch PA.

The patch PA according to the present application may absorb a substance from the plate PL by bringing the plate PL to be in contact with the patch PA. In this regard, in a contact area near a contact surface between the plate PL and the patch PA, a water film WF may be formed due to a liquid substance SB captured by the patch PA and/or a substance applied on the plate PL. When a water film (aquaplane) WF is formed in the contact area, the substance applied on the plate PL may be captured by the water film WF. The substance captured by the water film WF may freely flow within the patch PA.

When the patch PA is spaced and separated from the plate PL by a certain distance or more, the water film WF moves along with the patch PA, so that the substance applied on the plate PL may be absorbed by the patch PA. The substance applied on the plate PL may be absorbed by the patch PA, when the patch PA is spaced apart from the plate PL by a certain distance or more. When the patch PA and the plate PL are spaced apart and separated from each other, the liquid substance SB provided to the patch PA may not move to the plate PL, or only a small portion thereof may be absorbed by the patch PA.

All or part of the substance applied on the plate PL may react specifically with all or part of the substance captured in the patch PA. In this regard, the patch PA may selectively absorb a substance from the separate plate PL. This is the case when the patch PA has a stronger attractive force than the plate PL with respect to the portion of the substance captured in the patch PA.

For example, some substances may be fixed on the plate PL. In other words, some substances may be fixed on the plate PL, and other substances may not be fixed or may be applied with fluidity. In this regard, when the patch PA and plate PL contact each other and are separated from each other, only substances other than some fixed, among the substances applied to the plate PL, may be selectively absorbed by the patch PA. In contrast, the selective absorption may occur due to the polarity of the substance located on the plate PL and the substance captured by the patch PA regardless of whether or not the substance is fixed.

In an embodiment, in the case in which the liquid substance SB captured by the patch PA specifically binds to at least a portion of the substance applied to the plate PL, when the patch PA is brought in contact with and then separated from the substance applied to the plate PL, only at least a portion of the substances applied to the plate PL may be absorbed by the patch PA.

In an embodiment, a portion of the substance applied on the plate PL may react specifically with a substance that has been fixed on the plate PL in advance. In this case, from among the substance applied on the plate PL, only a portion other than a portion thereof that reacts specifically with the substance that has been fixed on the plate PL in advance, may be absorbed by the patch PA.

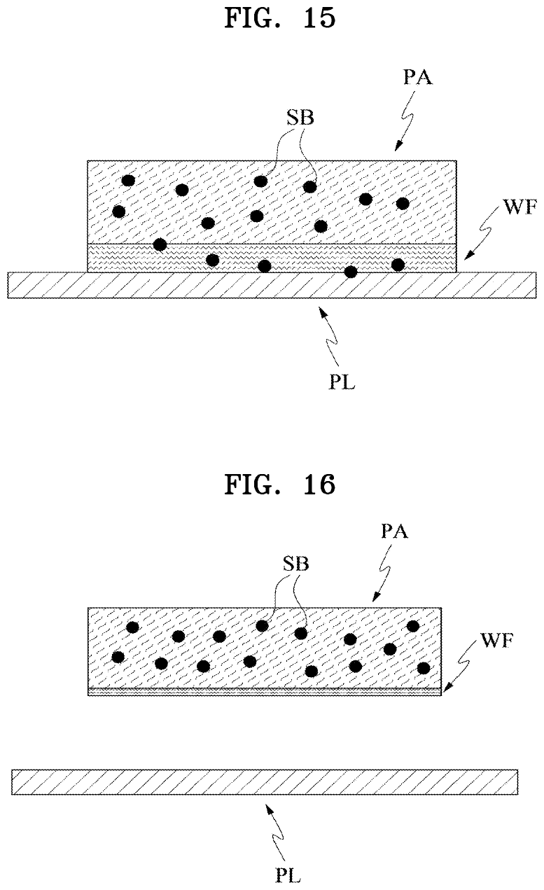

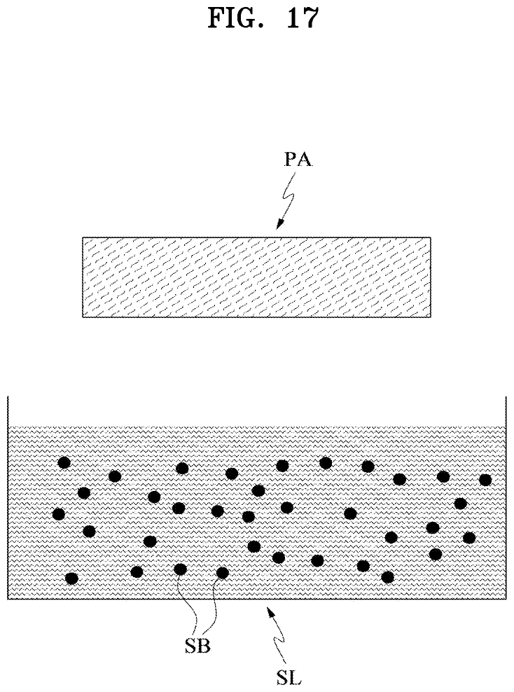

FIGS. 14 to 16 illustrate an example of the absorption of a substance by the patch PA from the external plate PL, as an example of functions of the patch PA according to the present application. Referring to FIGS. 14 to 16, the patch PA may absorb, from the external plate PL, a portion of the substance located on the external plate PL. The absorption of the substance may be performed based on the fact that the patch PA contacts the external plate PL, thereby forming a water film WF near the contact area between the external plate PL and the patch PA, and the water film WF enables the substance to move to the patch PA.

Herein, it is assumed that the substance is absorbed from the fluidity-having substance SL to the patch PA. The fluidity-having substance SL may be a liquid foreign substance that is contained in a separate container or that flows. In an embodiment, by providing an environment in which the fluidity-having substance SL flows into the liquid substance SB captured in the patch PA and vice versa, some or all of the fluidity-having substance SL may be absorbed by the patch PA. In this regard, such an environment may be formed by bringing the patch PA in contact with at least a portion of the fluidity-having substance SL.

When the patch PA comes into contact with the fluidity-having substance SL, the patch PA may be in such a state that enables the movement of a substance with the fluidity-having substance SL. When the patch PA is separated from the fluidity-having substance SL, at least a portion of the fluidity-having substance SL may be absorbed by the patch PA.

The absorption of a substance from the fluidity-having substance SL to the patch PA may depend on the difference between the concentration of the substance captured in the patch PA and the concentration of the fluidity-having substance SL. In other words, when the concentration of the additive substance AS in the liquid substance SB captured by the patch PA is lower than the concentration of the additive substance AS in the fluidity-having substance SL, the additive substance AS may be absorbed by the patch PA.

On the other hand, when a substance is absorbed from the fluidity-having substance SL to the patch PA, the absorption of the patch PA may be controlled by, in addition to depending on the concentration difference while in contact as described above, adding an electrical factor or changing the physical condition. Furthermore, a substance captured by the patch PA and a target absorption substance may be in indirect contact through a medium, not in direct contact, to proceed the absorption of a substance.

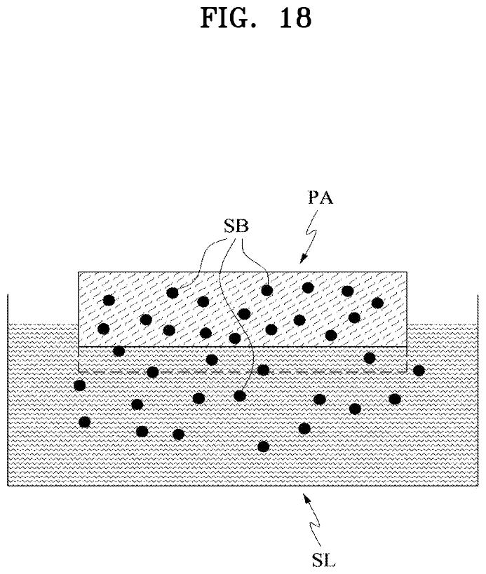

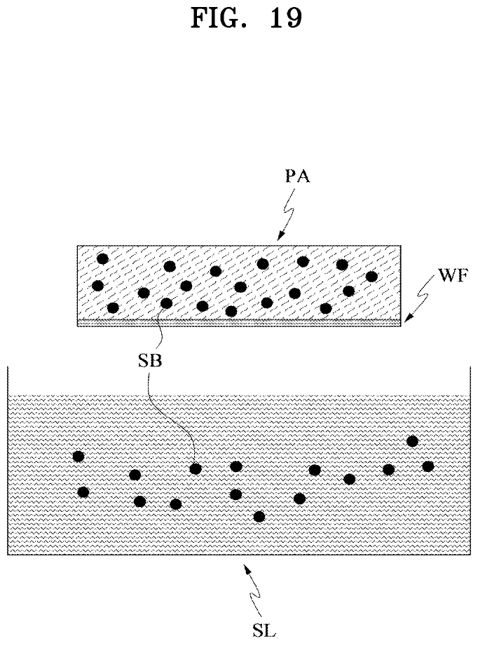

FIGS. 17 to 19 illustrate an example of the absorption of a substance by the patch PA from the fluidity-having substance SL, as an example of functions of the patch PA according to the present application. Referring to FIGS. 17 to 19, the patch PA may absorb a portion of the fluidity-having substance SL. The absorption of the substance may be performed based on the fact that the patch PA is added to the fluidity-having substance SL or is brought into contact with the fluidity-having substance SL, thereby enabling the liquid substance SB captured by the patch PA to flow into the fluidity-having substance SL or vice versa.

Herein, it is assumed that the patch PA absorbs a foreign substance from the other patch PA.

The absorption of a foreign substance by the patch PA from the other patch PA may be performed based on a difference between the bonding force of the absorbed foreign substance and a substance captured by the patch PA and the bonding force of the absorbed foreign substance and the foreign substance that is not absorbed by the patch PA. For example, when the absorbed substance has hydrophilicity, the patch PA has hydrophilicity, and the attractive force of the absorbed substance and the patch PA is stronger than the attractive force between the other patch PA and the absorbed substance (i.e., when the patch PA has a stronger hydrophilic property than the other patch PA), in the case where the patch PA and the other patch PA are separated after being brought in contact, at least a portion of the foreign substance may be absorbed by the patch PA.

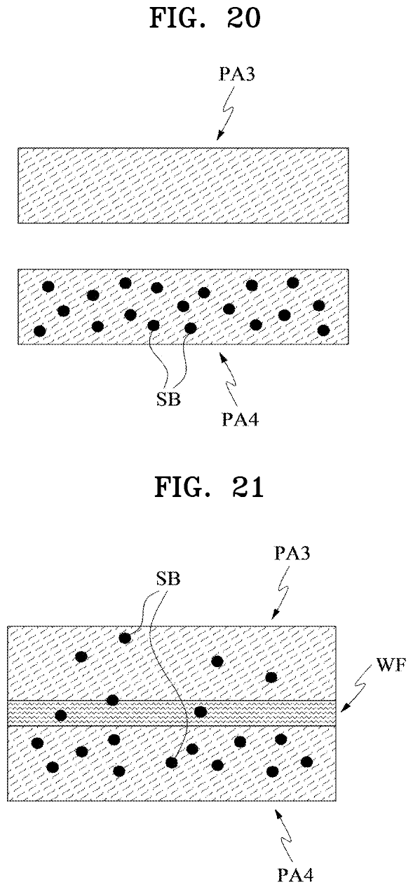

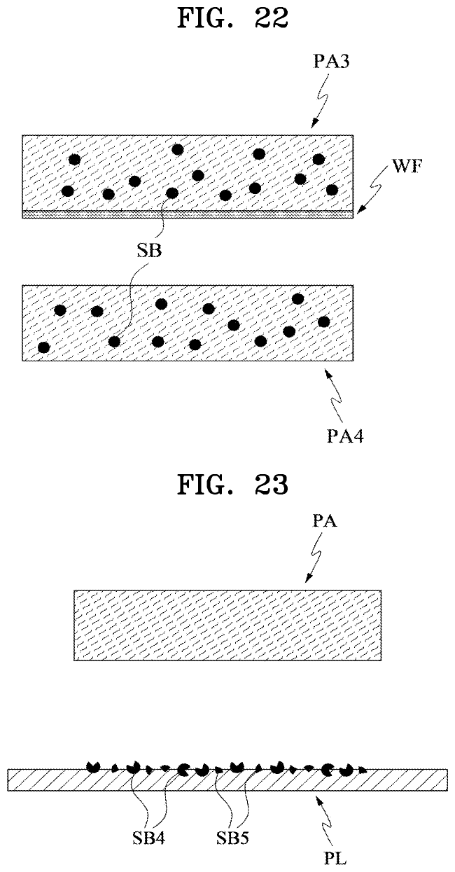

FIGS. 20 to 22 illustrate an example of the absorption of a substance by a patch PA3 from another patch PA4, as an example of functions of the patch PA according to the present application. Referring to FIGS. 20 to 22, the patch PA3 may absorb a substance that has been located in the other patch PA4. The absorption of the substance may be performed based on the fact that due to the contact between the patch PA3 and the other patch PA4, the liquid substance SB captured by the patch PA3 are exchanged with the liquid substance SB captured by the other patch PA4.

On the other hand, according to the ratio of the frame structure of the three-dimensional net structure NS constituting the patch PA to the total volume of the patch PA, the binding force of the patch PA to the absorbed foreign substance may change. For example, as the volume ratio of the frame structure to the patch PA increases, the amount of substance captured by the structure may be decreased. In this case, since the contact area between the substance captured by the patch PA and the target material is decreased, the bonding force between the patch PA and the target material may be decreased.

In this regard, in the fabrication step of the patch PA, the polarity of the patch PA may be controlled by adjusting the ratio of the material constituting the net structure NS. For example, in the case of patch PA manufactured using agarose, the concentration of the agarose may be adjusted to control the degree of the absorption.

When the separate area has a weaker bonding force with respect to the substance provided from the patch PA than the patch PA and the patch PA and the other patch PA are brought in contact and then separated, the absorbed foreign substance may be separated, together with the patch PA, from the other patch PA.

2.2.4.3 Providing of the Environment

The patch PA according to the present application may perform the function of adjusting the environmental conditions of a target area according to characteristics described above. The patch PA may provide a target area an environment derived from the patch PA.

The environmental conditions derived from the patch PA may depend on the liquid substance SB captured by the patch PA. The patch PA may provide a target environment to a substance located in an external area, according to characteristics of a material placed by the patch PA or to correspond to characteristics of the substance placed in the patch PA.

The adjusting the environment may be understood as changing the environmental conditions of the target area. The changing of the environmental condition of the target area may be implemented in such a manner that the area affected by the patch PA is expanded to include at least a part of the target area or the environment of the patch PA is shared with the target area.

Hereinafter, the function of the patch PA as described above will be referred to as the environment, for convenience.

The providing of the environment by the patch PA may be performed when a substance flows between the patch PA and an external area. The providing of the environment by the patch PA may be performed by contact. For example, when the patch PA contacts a target area (for example, a foreign substance, a plate PL, etc.), the target area may be provided with a specific environment by the patch PA.

The patch PA may adjust the environment of a target area TA by providing an appropriate level of pH, osmotic pressure, humidity, concentration, temperature, or the like. For example, the patch PA may impart liquidity to a target area TA or a target material. This imparting of liquidity may occur with movement of at least a portion of the substance captured by the patch PA. A wetting/moist environment may be provided to the target area TA through the liquid substance SB captured by the patch PA or the base substance BS.

The environmental factors provided by the patch PA may be kept constant according to the purpose. For example, the patch PA may provide homeostasis to the target area. As another example, as a result of the providing of the environment, the environmental conditions of the target area may be adapted to the substance captured by the patch PA.

The providing of the environment by the patch PA may be the result of diffusion of the liquid substance SB contained in the patch PA. That is, when the patch PA and the target area are in contact, the substance may be moved through the contact area formed by the contact. In this regard, an environment change due to osmotic pressure according to the diffusion direction of the substance, an environment change according to an ion concentration, a wet environment, and a change in PH may be implemented.

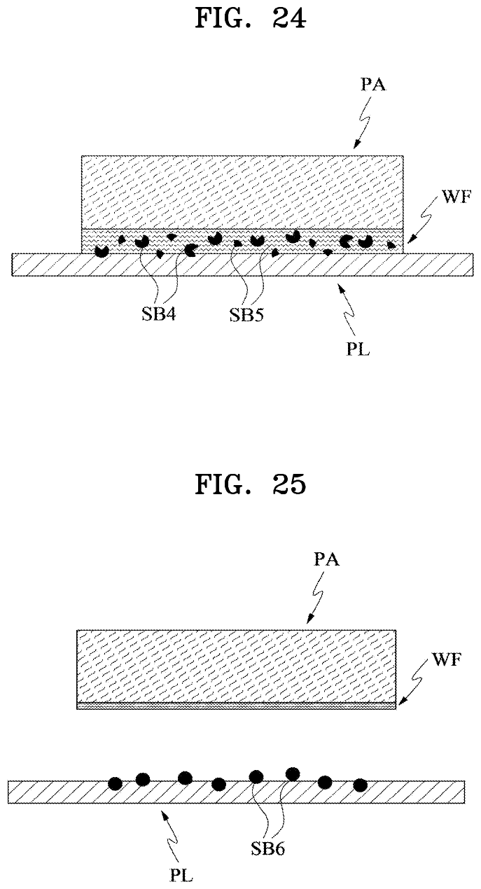

FIGS. 23 to 25 illustrate the providing of a certain environment to the external plate PL by the patch PA, as an example of the providing of an environment from among functions of the patch PA according to the present application. Referring to FIGS. 23 to 25, the patch PA may provide a certain environment to the external plate PL on which a fourth substance SB4 and a fifth substance SB5 are located. For example, the patch PA may provide the plate PL a certain environment in which the fourth substance SB4 and the fifth substance SB5 are reacted to each other to form a sixth substance SB6. The providing of the environment may be achieved by forming a water film WF near the contact area where the patch PA contacts the plate PL and capturing the fourth substance SB4 and the fifth substance SB5 in the water film WF.

### 3. Patch Application

The patch PA according to the present application, may be implemented to perform various functions by appropriately applying the functions of the patch PA.

Hereinafter, the technical idea of the present application will be described with reference to some embodiments. However, the technical range to which the function of the patch PA according to the present application is used or applied should be expanded and interpreted within the range of easy derivation by those skilled in the art, and the invention scope defined according to the present application should not limited by the embodiments described in this specification.

3.1 In-Patch

The patch PA may provide a reaction area of a substance. In other words, a reaction of the substance may occur in at least a portion of the spatial area affected by the patch PA. In this regard, the reaction of a substance may be a reaction between liquid substances SB captured by the patch PA and/or a reaction between the liquid substance SB captured and a substance provided from outside the patch PA. The providing of a reaction area of a substance may be activating or promoting the reaction of the substance.

In this regard, the liquid substance SB captured by the patch PA refers to at least one of a substance that is injected at the time of manufacture of the patch PA, a substance that is added to the patch PA after manufacture, and contained in the patch PA, and a substance that is temporarily captured by the patch PA. In other words, any substance that is captured by the patch PA at the time when the reaction in the patch PA is activated, may react in the patch PA regardless of in what form the substance is captured by the patch PA. Furthermore, it is possible that a substance introduced after the manufacture of the patch PA acts as a reaction initiator.

The providing of a reaction area for a reaction involving a liquid substance SB captured by the patch PA may correspond to an example of the smaller concept of section 2.1.3 (that is, providing of a reaction space). Alternatively, the providing may correspond to a multi-concept of the combination of the functions described in section 2.1.3 and section 2.2.4.2 (that is, absorption). Further, embodiments of the present disclosure is not limited thereto, and two or more other ( [symbol] ) functions may be implemented in combination.

### 3.1.1 First Embodiment

Hereinafter, it is assumed that the absorbing function by the patch PA and the providing function of the reaction space (hereinafter, referred to as providing function) are performed by one patch PA. In this regard, the absorbing function and the providing function may be performed at the same time, at separate points of time, or sequentially performed to perform one other function. On the other hand, it may be considered that the patch PA further includes, in addition to the absorbing and providing functions, other functions in addition to the present embodiment.

As described above, the patch PA may perform the function of capturing a substance, and even when the substance is captured, liquidity may exist. When the distribution of some components of the liquid substance SB is non-uniform, the non-uniform component may diffuse.

Even when the components of the liquid substance SB are uniformly distributed, the liquid substance SB may be in a state of having a certain level of mobility due to irregular motion of the particles. In this regard, reactions between substances, such as specific binding between substances, may occur within the patch PA.

For example, in the patch PA, in addition to the reaction between the captured substances, a reaction between a fluidity-having substance newly captured by the patch PA and a substance that has been captured in the patch PA, that is, a specific binding therebetween, may occur.

The reaction between the fluidity-having substance and the captured substance may be performed in a place which is separated from an arbitrary space in which the fluidity-having substance has been provided. For example, after the patch PA absorbs the fluidity-having substance from an arbitrary space, the patch PA is separated from the arbitrary space, and the reaction between the absorbed substance and the substance that has been captured by the patch PA may occur in the patch PA.

In addition, the patch PA may cause a reaction of the captured substance by performing the absorbing function on the fluidity-having substance. In other words, a reaction between the absorbed substance and the substance captured by the patch PA may occur by triggering the absorption of the fluidity-having substance of the patch PA. The reaction may be carried out inside the space defined by the patch PA.

In addition, due to the reaction occurring inside the patch PA, the composition of the liquid substance SB captured by the patch PA may be changed. In particular, when the substance captured inside the patch PA is a compound, the chemical composition thereof may change before and after the reaction. In some embodiments, the distribution of the composition according to the position of the substance in the patch PA may be changed. This may be due to, for example, diffusion or by particles having a specific attraction with respect to other substances.

When the composition of the liquid substance SB is changed due to the reaction inside the patch PA, due to the difference between the concentration of the patch PA and the concentration of a substance outside the patch PA (in the case where is a substance in contact, the substance in contact), a portion of the substance may be absorbed by the patch PA, or the substance may be released from the patch PA to the substance outside the patch.

### 3.1.2 Second Embodiment

Hereinafter, an embodiment in which the storage function and the function of providing a reaction space for the substance of the patch PA are performed together for at least a predetermined time will be described. In an embodiment, the patch PA may perform the function of providing a space for the reaction of at least a portion of the liquid substance SB contained in the patch PA .

The patch PA may contain a substance and provide a reaction space for the contained substance. In this regard, the reaction space provided by the patch PA may be the micro-cavities formed by the net structure NS of the patch PA or the surface area of the patch PA. In particular, when a substance contained in the patch PA and a substance applied to the surface of the patch PA react together, the reaction space may be a surface area of the patch PA.

The reaction space provided by the patch PA may provide specific environmental conditions. The patch PA may adjust the environmental conditions of the reaction while the reaction in the liquid substance SB located in the patch PA is in progress. For example, the patch PA may function as a buffer solution.

The patch PA contains the substance through the net structure, so that a separate storage container is not needed. In addition, when the reaction space of the patch PA is the surface of the patch PA, observations may be made only through the surface of the patch PA. To this end, the shape of the patch PA may be modified and designed into a shape that is easy to observe.

The liquid substance SB contained in the patch PA may be denatured or react with other types of substances. The composition of the liquid substance SB contained in the patch PA may be changed over time.

Meanwhile, the reaction may refer to a chemical reaction in which the chemical formula is changed, or a change in a physical state or a biological reaction. In this regard, the liquid substance SB contained in the patch PA may be a single-component substance or a mixture containing a plurality of components.

### 3.2 Channeling

Hereinafter, a description will be given of a patch PA that performs a function of providing a movement path of a substance. In an embodiment, the patch PA may capture, absorb, release, and/or contain the fluidity-having substance as described above or the like. By using each or combination of the functions of the patch PA, various embodiments of the patch PA that perform a function of providing a movement path of a substance may be implemented. However, to help understanding, some embodiments will be disclosed.

### 3.2.1 Third Embodiment

The patch PA may be implemented to perform the functions described in section 2.2.4.1 (that is, the delivery function) and section 2.2.4.2 (that is, the absorbing function) among the functions of the patch PA described above. In this regard, the absorbing function and the delivery function may be provided together, and may be provided sequentially.

The patch PA may provide the movement path for the substance by performing the absorbing function and the delivery function together. In particular, the movement path of the foreign substance may be provided by absorbing a foreign substance and delivering the same to an external area.

The providing of a movement path for a foreign substance by the patch PA may be performed by absorbing the foreign substance and releasing the foreign substance. In an embodiment, the patch PA may contact the foreign substance to absorb the foreign substance and contact the external area to deliver the foreign substance to the external area. In this regard, the patch PA captures the foreign substance and delivers the same to the external area may proceed in a similar process to those described in connection with the absorbing function and the delivery function as described above.

The foreign substance absorbed and delivered to the patch PA may be liquid or solid.

Accordingly, the patch PA may allow a portion of the foreign substance to be delivered to another foreign substance. The patch PA, the foreign substance, and the other foreign substance may be in contact at the same time. The time when the patch PA contacts the foreign substance may be different from the time when the patch PA contacts the other foreign substance.

The time when the patch PA contacts the foreign substance may be different from the time when the patch PA contacts the other foreign substance. In the case where each of the foreign substances is brought in contact at different times, the patch PA contacts the foreign substance, the foreign substance is separated from the patch PA, and then, the patch PA contacts the other foreign substance. In this regard, the patch PA may temporarily contain a substance captured from the foreign substance.

The patch PA may additionally provide a time delay while providing a movement path for the substance. In addition, the patch PA may appropriately control the delivery amount and delivery rate of a substance to the other foreign substance.

Meanwhile, such a series of processes may be performed in one direction based on the patch PA. As an example, the absorption of the substance is made through one side of the patch PA, an environment may be provided to the inner space of the patch PA, and the substance may be released through the other side thereof facing the one side.

### 3.2.2 Fourth Embodiment

The patch PA may absorb and release a substance, among the functions of the patch PA, and at the same time provide a reaction space for the substance. In this regard, the absorption, release and provision of reaction space of the substance may be performed simultaneously or sequentially.

According to one embodiment, the patch PA may provide a reaction space to the absorbed foreign substance for at least a certain period of time, in performing the process of absorbing and releasing of the foreign substance. The patch PA may provide, for at least a certain period of time, a specific environment to the liquid substance SB captured by the patch PA containing the absorbed foreign substance.

The liquid substance SB that has been captured by the patch PA and the foreign substance captured by the patch PA may react inside the patch PA. The foreign substance absorbed in the patch PA may be affected by the environment provided by the patch PA. The substance released from the patch PA may contain at least some of the substance produced through the reaction. The foreign substance may be released from the patch PA after the composition, properties, etc. thereof are changed.

The absorbed substance may be released from the patch PA. The absorbing of the foreign substance by the patch PA and the release thereof from the patch PA may be understood as passing through the patch PA. The foreign substance that has passed through the patch PA may lose the identity thereof due to a reaction inside the patch PA or an influence of the environment provided by the patch PA.

The absorption of the foreign substance, the reaction of a substance, and the delivery of a substance may proceed in one direction. In other words, the absorbing of the substance may be performed at one position of the patch PA, the providing of the environment may be performed at one position, and the release of the substance may be performed at one position.

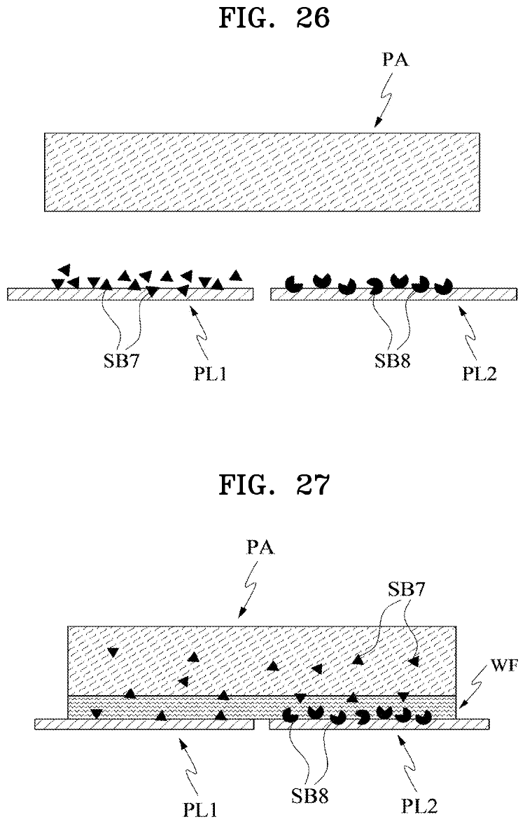

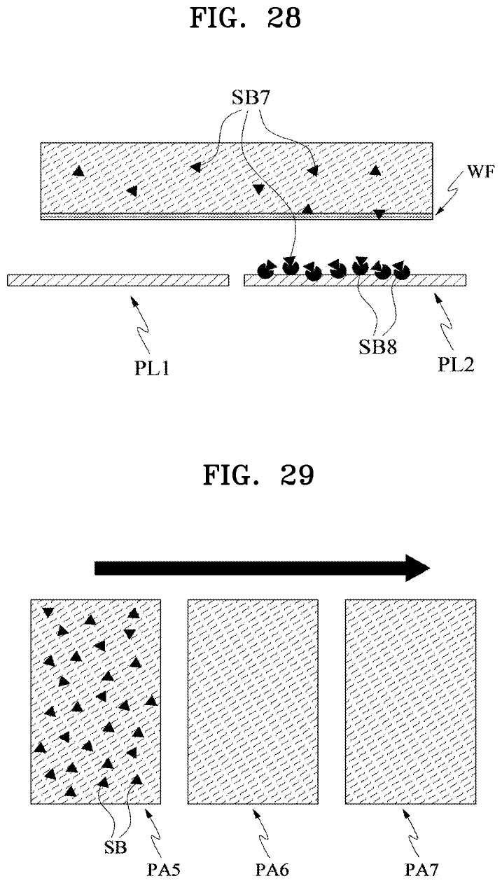

FIGS. 26 to 28 illustrate providing a movement path of a substance between two plates PL, as an embodiment of the patch PA according to the present application. Referring to FIGS. 26 to 28, the patch PA may provide a movement path of the substance between the plate PL1 on which a seventh substance SB7 is applied and the plate PL2 on which an eighth substance SB8 is applied. For example, when the seventh substance SB7 has a binding property with respect to the eighth substance SB8, and the eighth substance SB8 is fixed on the plate PL2, the seventh substance SB7 may move through the patch PA and bind with the eighth substance SB8 by bringing the patch PA in contact with the plates PL1 and PL2. The connection between the seventh substance SB7 and the eighth substance SB8 with the patch PA may be due to the water film WF formed when the patch PA is brought in contact with each of the plates PL1 and PL2.

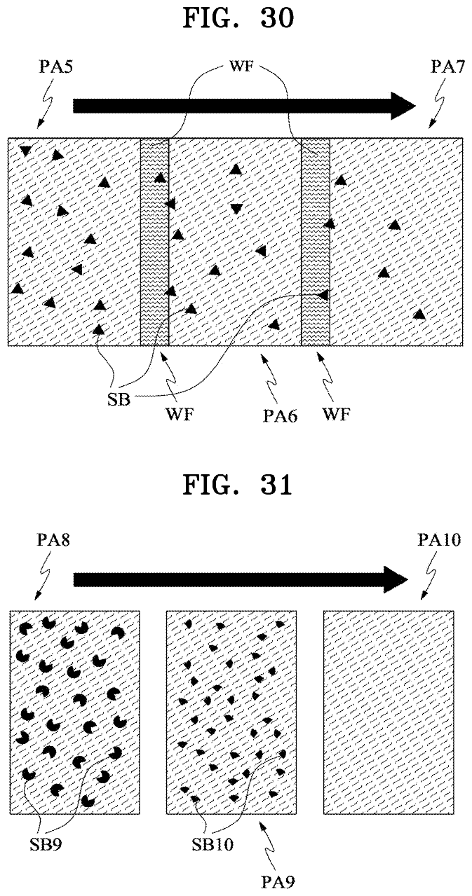

FIGS. 29 and 30 illustrate providing a movement path of a substance between two patches, as an embodiment of the patch PA according to the present application. Referring to FIGS. 29 and 30, a patch PA6 providing the movement path may be in contact with a patch PA5 containing a target material to be moved and a patch PA7 receiving the target material. The target material may be moved to the patch PA7 that receives the target material by bringing the patch PA6 in contact with the patch PA5 providing the movement path and the patch PA7 containing the target material. The movement of the substance between respective patches may occur through the water film WF formed near the contact area between the patches.

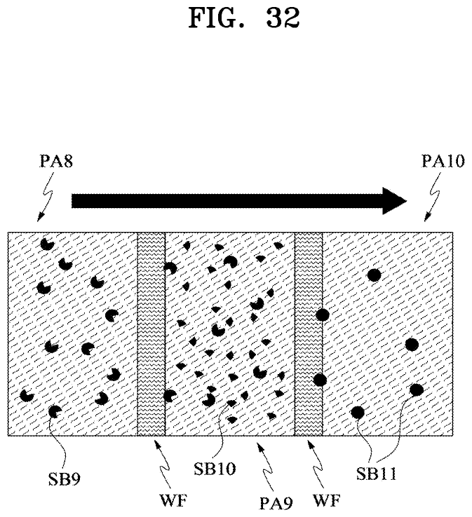

FIGS. 31 and 32 illustrate providing a movement path of a substance between two patches, as an embodiment of the patch according to the present application. Referring to FIGS. 29 and 30, a patch PA9 providing the movement path may be in contact with a patch PA8 containing a ninth material SB9 and a patch PA10 receiving the substance. The patch PA9 providing the movement path may absorb the ninth material SB9 by contacting the patch PA8 containing the ninth material SB9. The absorbed ninth material SB9 may react with a tenth substance SB10, which has been contained in the patch PA9 providing the movement path, to form an eleventh substance SB11. The eleventh substance SB11 may be delivered to the patch PA10 receiving the substance from the patch PA9 providing the movement path.

The movement of the substance between respective patches PA may occur through the water film WF formed near the contact area between the patches PA.

### 3.3 Multi Patch

The patch PA may be used alone, and also, a plurality of patches PA may be used together. In this regard, the wording “a plurality of patch PAs may be used together” includes a case where the patches PA are used simultaneously as well as a case where the patches PA are used sequentially.

When the patches PA are used at the same time, the respective patches PAs may perform different functions. Each patch PA of the patches PA may contain the same substance, but may contain different substances.

When the patches PA are used at the same time, the movement of the substance among the patch PAs may not occur because the respective patches PA are not in contact with each other. In some embodiments, in the state in which substances contained in the respective patches PA are mutually exchangeable, a target function may be performed.

The patches PA used together may be manufactured in a shape similar to each other or with the same standard, but the patches PA having different shapes may also be used together. In addition, the respective patches PA may have different densities of the net structure NS or different components constituting the net structure NS.

### 3.3.1 Multiple Patch Contact

In the case of using the patches PA, the patches PA may contact one target area TA. The patches PA may contact one target area TA to perform a target function.

The patches PA may contact different target areas TA when there are multiple target areas TA. When there are multiple target areas TA, the patches PA may perform a target function by respectively contacting corresponding target areas TA.

The patches PA may be in contact with the substance applied to the target area TA. In this regard, the substance applied to the target area TA may be fixed or have liquidity.

The target function may be a delivery or absorbing function of a substance. However, each patch PA does not have to deliver the same substance or absorb the same substance. In some embodiments, the patches PA may deliver different substances to the target area TA or absorbs different components from a substance located in the target area TA.

The target function may vary depending on a patch PA constituting the patches PA. For example, one patch PA may perform a function of delivering a substance to a target area TA, and the other patch PA may perform a function of absorbing a substance from the target area TA.

The patches PA contains different substances, and the different substances may be delivered to one target area TA and used to induce a target reaction. When a multi-components substance is required to induce the target reaction, the multi-components substance may be respectively contained in a plurality of patch PAs and delivered to the target area TA. The use of such patches PA may be particularly useful in cases where the properties of the substance required for the target reaction are lost or deteriorated when the substance required for the reaction is mixed due to, for example, being contained in a single patch PA.

According to an embodiment, when the patches PA contain substances of different components and the substances of the different components have different specific binding relationships, the substances of the different components may be delivered to the target area TA. The patches PA may be used to detect a plurality of specific binding from a substance applied to the target area TA by delivering substances of the different components.

According to some embodiments, each of the patches PA contains a substance of the same component, and the patches PA may have different concentrations of the substance of the same component. The patches PA containing substances of the same component may be used to determine the influence of the concentration of the substance contained in the patches PA by being in contact with the target area TA.

On the other hand, in the case of using the patches PA as described above, a bundle of patch PAs may be modified in an efficient form before use. In other words, the configuration of the patches PA to be used may be used differently each time when the patches PA are implemented. That is, the patches PA may be used in the form of a cartridge. In this regard, the shape of each patch PA used may be properly standardized.

The patch PA in the form of a cartridge may be suitable when patches PA containing different materials are manufactured and selected according to the purpose.

In particular, in the case of detecting a specific reaction of each substance from the target area TA using multiple types of substances, whenever the detection is performed, the combination of the specific reaction to be detected may be changed.

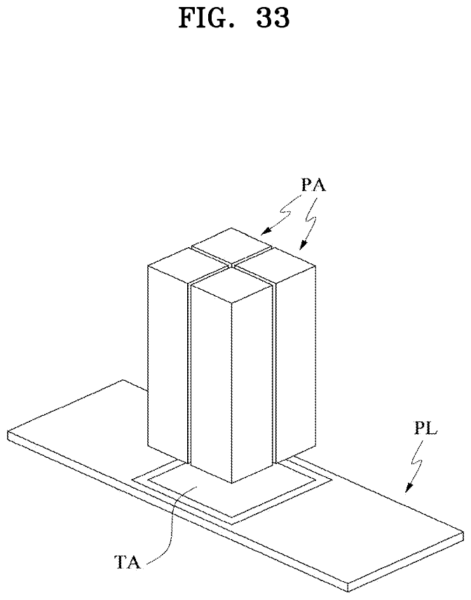

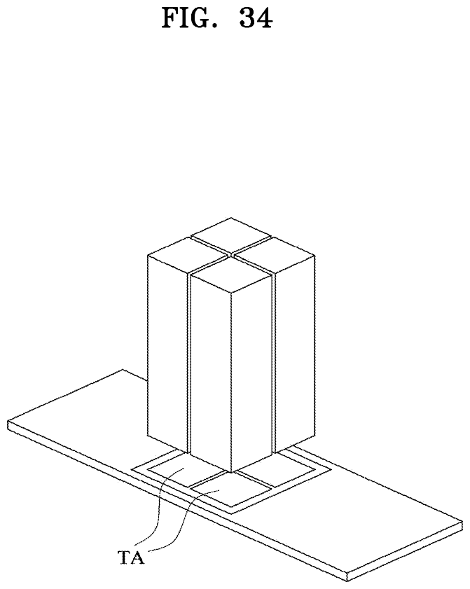

FIG. 33 illustrates a case where a plurality of patches PA are used together, as an embodiment of a patch PA according to the present application. According to FIG. 33, the patches PA according to an embodiment of the present application may be in contact with the target area TA located on the plate PL at the same time. Each patch PA constituting the patches PA may have a standardized form. The patches PA contain a first patch and a second patch and the substance contained in the first patch may be different from the substance contained in the second patch.

FIG. 34 illustrates an embodiment in which a plurality of patches PA are used together, and the plate PL may include a plurality of target areas TA. According to FIG. 34, the patches PA according to an embodiment of the present application may be in contact with the target areas TA located on the plate PL at the same time. The patches PA may include a first patch PA and a second patch PA, the target areas TA may include a first target area and a second target area, and the first patch may contact the first target area and the second patch may contact the second target area.

### 3.3.2 Fifth Embodiment

The patches PA may perform a plurality of functions. As described above, the respective patches PA may perform a plurality of functions at the same time, and in an embodiment, the respective patches PA may perform different functions at the same time. However, the present embodiment is not limited to the above case, and the respective functions may be performed in combination in the patches PA.

First, as a case where the respective patches PA perform a plurality of functions at the same time, each patch PA may perform both storage and release of the substance. For example, the respective patches PA may contain different substances and release the respective substances to the target area TA. In this case, the respective contained substances may be released at the same time or sequentially.

As a case where the respective patches PA perform different functions at the same time, only some of the respective patches PA may perform storage and release of the substance. In this case, only some of the respective patch PAs may contact the target area TA and release a substance to the target area TA.

### 3.3.3 Sixth Embodiment

When a plurality of patches PA are used, as described above, the patches PA may perform a plurality of functions. First, each patch PA may perform containing, releasing and absorbing of the substance at the same time. In an embodiment, some of the patch PA may perform the storage function on the substance, others thereof may perform the release function on the substance, and others thereof may perform the absorption function on the substance. However, the present embodiment is not limited to the above case, and the respective functions may be performed in combination in the patches PA.

For example, at least some of the patches PA may contain a substance and release the contained substance to the target area TA. In this regard, at least others of the patches PA may absorb the substance from the target area TA. Some of the patches PA may release a substance that specifically binds to a substance located in the target area TA. In this regard, the detection of specific binding may be performed by absorbing a substance that does not form the specific binding from among the substances located in the target area TA using another patch PA.

### 3.3.4 Seventh Embodiment

When a plurality of patches PA are used, the respective patches PA may perform storage and release of the substance and providing of the environment to the substance, at the same time. In an embodiment, some of the patch PA may perform the storage function on the substance, others thereof may perform the release function on the substance, and others thereof may perform the providing of the environment to the substance. However, the present embodiment is not limited to the above case, and the respective functions may be performed in combination in the patches PA.

For example, one of the patches PA may release the contained substance into the target area TA. In this regard, another patch PA may provide an environment to the target area TA. In this regard, the providing of the environment may be implemented by delivering the environmental condition of the substance contained in the other patch PA to the target area TA. In an embodiment, a reactive substance may be provided to the target area TA by one patch PA, and the other patch PA may provide a buffer environment by being in contact with the target area TA.

In an embodiment, the patches PA may be in contact with each other. In this regard, at least one patch PA may contain the substance and release the contained substance to the other patch PA providing the environment. In the present embodiment, the patch PA providing the environment may contact each of at least two patch PA that are not in contact with each other and releases a substance, and absorbs a substance from the respective patches PA.

Hereinafter, a case of testing a specimen using the above-described patch will be described. Specifically, embodiments will be described about a method and apparatus in which, in delivering a reagent to a specimen by using the patch, the reagent is efficiently delivered to the specimen, and the test accuracy of the specimen by using the reagent is improved.

### 4. Performing Tests

The patch disclosed in this specification may be used for testing a biological sample. By performing a test of a biological sample using the patch, the accuracy of the test may be improved while preventing waste of reagents used for the test and minimizing damage to the biological sample.

Hereinafter, a method of performing a test of a biological sample using a patch disclosed in this specification will be described.

### 4.1 Test Process

According to an embodiment of the disclosure disclosed in the present specification, the biological sample may be tested using a patch that contains a reagent used for testing the biological sample. Hereinafter, some embodiments of a method of performing a test of the biological sample using a patch containing the reagent will be described.

The test method for the biological sample according to an embodiment of the present disclosure may include preparing a patch containing the reagent and a biological sample as a test target, and providing the reagent to the biological sample.

The test method for the biological sample may be used for testing of various biological samples.

For example, the biological sample may be a liquid sample. For example, the biological sample may be a body fluid sample such as blood, urine, saliva, or a cell suspension. The biological sample to be tested may be spread on the substrate of a surface. The biological sample may be fixed on the surface of the substrate. The biological sample may be spread on the surface of the substrate on which an antibody is fixed.

In an embodiment, the biological sample may be in a solid phase. The biological sample may be a tissue sample or cultured cells. The biological sample may be prepared as a section. The biological sample may be prepared as a paraffin-filled tissue section or a frozen tissue section.

However, the application of the test method disclosed in this specification is not limited to the test of a biological sample, and may be generally applied when a reagent is delivered to test a target material.

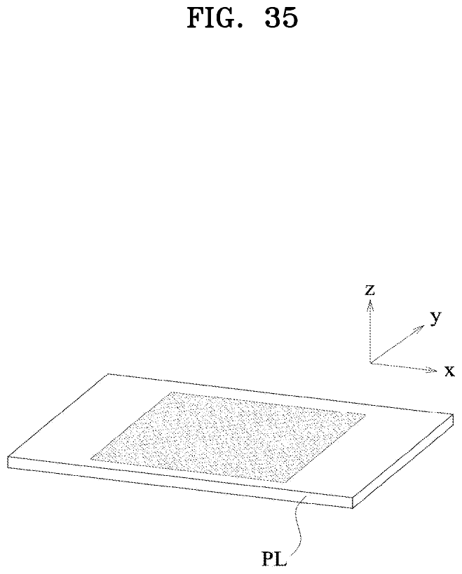

FIG. 35 illustrates examples of a substrate and a biological sample disclosed in the present specification. The substrate may have a width direction, a length direction and a height. The width, length, and height directions of the substrate may be defined as an X-axis direction, a Y-axis direction, and an Z-axis direction, respectively. Hereinafter, the X-axis direction, Y-axis direction, and Z-axis direction are considered to be determined based on the substrate as shown in FIG. 35 unless defined otherwise.

The test method for the biological sample may be performed by using the patch that contains at least one type of various kinds of reagents used for testing the biological sample.

The patch may contain a treatment reagent for changing the biological sample to be suitable for a test. For example, the patch may contain a fixing reagent that fixes the biological sample. The patch may contain a buffer reagent that adjusts the pH of the biological sample.

The patch may contain a detection reagent for detecting a target material included in the biological sample. The patch may contain many kinds of detection reagents for detecting many kinds of target materials.

The patch may contain a detection reagent for detecting a target protein. The patch may contain an antibody for detecting a target antigen included in the biological sample. The patch may contain a probe for detecting the target generic substance.

The detection reagent may be a staining reagent for staining and labeling the target material.

For example, the staining reagent may be a staining reagent for Romanowsky staining, such as a Giemsa staining reagent, a Wright staining) reagent, a Giemsa-Wright staining reagent , or the like.

In some embodiments, the staining reagent may be methylene blue, carmine acetate, eosin, acid fuchsin, safranin, Janus green B, hemotoxylin, and the like.

The staining reagent may contain a substance that decolorizes or mordants cells in addition to a substance that directly stains cells. For example, in the case of performing Gram staining, a patch PA that contains crystal violet as the main staining agent, a patch PA that contains safranin as a contrast staining agent, a patch PA that contains iodine as a mordant, and a patch PA that contains alcohol, which is the bleaching agent may be prepared.

The detection reagent may be a fluorescent reagent for fluorescent-labeling the target material. For example, the patch may contain, for example, an antibody with a fluorophore that binds to the target material and emits fluorescent light as a product.

According to an embodiment of the disclosure disclosed in this specification, the test method for the biological sample may include providing a reagent contained in the patch to the biological sample by using the patch. The test method for the biological sample may include delivering at least some of the reagent contained in the patch to the biological sample by bringing the patch containing the reagent in contact with the biological sample and then separating the patch from the biological sample.

The bringing the patch in contact with the biological sample may be such that one surface of the patch is located close to the biological sample so that the patch is connected to the biological sample. The bringing the patch in contact with the biological sample may be placing the patch to be close to the biological sample. The bringing the patch in contact with the biological sample may be pressing the patch toward the biological sample such that a certain level of force is applied to the biological sample.

The test method for the biological sample may include forming a water film in an area where the biological sample contacts the patch by bringing the patch in contact with the biological sample. According to the test method for the biological sample, due to the contact with the patch, the biological sample may be connected to the patch through the water film. According to the test method for the biological sample, the reagent contained in the patch may be provided to the biological sample through the formed water film.

The test method for the biological sample may include delivering the reagent to the biological sample by spacing the patch from the biological sample. The test method for the biological sample may include delivering the reagent to the biological sample by separating, from the biological sample, at least a portion of the formed water film, together with the patch. In this regard, among the reagent provided to the biological sample from the patch through the water film, only such an amount of the reagent that is necessary for testing the biological sample may remain in the biological sample.

Meanwhile, in general, the description of ‘the delivery function’ described above with reference to FIGS. 5 to 7, among the patch functions, may be applied to the delivery of the reagent contained in the patch to the biological sample.

The test method for the biological sample according to an embodiment of the present disclosure may include preparing a patch containing the reagent and a biological sample as a test target, and bringing the patch in contact with the biological sample.

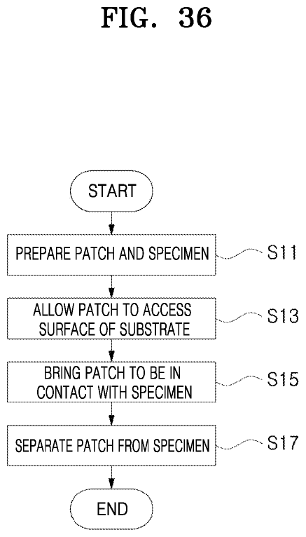

FIG. 36 illustrates a flowchart of a test method according to an embodiment of the present disclosure. Referring to FIG. 36, the test method according to an embodiment of the present disclosure may include preparing the patch and the biological sample (S11), allowing the patch to access the surface of a substrate (S13), and bringing the patch to be in contact with the biological sample (S15), and separating the patch from the biological sample (S17).

The test method for the biological sample may include preparing a patch for containing the reagent on one side of the biological sample (S11). The patch may be prepared such that one side thereof faces the surface of the substrate on which the biological sample is located. The patch may be located above the biological sample, and may have a lower surface facing the biological sample.

The test method for the biological sample may include allowing the patch to access the biological sample (S13). The test method for the biological sample may include lowering the patch toward the substrate on which the biological sample is located. The allowing the patch to access the biological sample may be changing the relative position of the patch with respect to the biological sample.

The test method for the biological sample may include bringing the patch in contact with the biological sample (S15). The test method for the biological sample may include bringing the patch in contact with the biological sample so that a water film is formed in the contact area between the patch and the biological sample. The patch may be kept in contact with the biological sample for a certain period of time.

The test method for the biological sample may include separating or spacing the patch from the biological sample (S17). The spacing of the patch from the biological sample may be changing the relative position of the patch with respect to the biological sample.

The spacing of the patch from the biological sample may be moving the patch upward away from the specimen located under the patch.

The spacing the patch may include spacing the patch from the biological sample such that, together with the patch, at least a portion of the water film formed in the contact area formed when the patch is in contact with the biological sample, is separated from the biological sample.

### 4.2 Test Device

The present specification provides a test device for performing a test of the biological sample by using a patch that contains a reagent used for testing of the biological sample.

The test device may include a patch receiving unit for receiving the patch, a substrate fixing unit for fixing a substrate on which a biological sample to be tested is located, and a control unit.

The patch receiving unit may receive the patch to be partially exposed. The patch receiving unit may be provided in a shape corresponding to the shape of the patch. The patch may be manufactured in a plate shape having a rectangular cross section, and the patch receiving unit may receive the patch such that one surface of the patch in the plate shape is exposed. The patch receiving unit may be provided to enable vertical and/or horizontal movement within a predetermined range therein. The patch receiving unit may raise and lower along a guiding member for guiding the movement of the patch receiving unit such that the patch contacts a biological sample located on the substrate at a predetermined position.

The position of the patch receiving unit may be changed directly or indirectly according to the driving of a driving unit providing the power. The patch receiving unit may be a patch receiving block to be described later. The patch receiving block may change the position and/or posture thereof directly according to the driving of the driving unit. The patch receiving unit may be a patch receiving member to be described later. The patch receiving member may change the position and/or posture thereof directly according to the driving of the driving unit. The receiving member may contact a pressing head whose position and/or posture is directly changed according to the driving of the driving unit, and due to the change in the position and/or posture of the pressing head, the position and/or posture of the receiving member may be changed.

The substrate fixing unit may fix the substrate at a predetermined position. The substrate may be provided as a slide glass or the like. The substrate fixing unit may fix the substrate so that a sample area in which a biological sample included in the substrate is located is at a predetermined position.

The substrate fixing unit may be provided to enable vertical and/or horizontal movement within a predetermined range therein. The substrate fixing unit may be moved up and down along a guiding member that guides the movement of the substrate fixing unit such that a biological sample located on the substrate contacts the patch at a predetermined position.

The test device may further include a control unit. The control unit may control the position of the substrate fixing unit or the patch receiving unit. The control unit may control the relative position of the substrate and the patch.

The control unit may control the patch receiving unit or the substrate fixing unit such that the patch is positioned on one side of the biological sample. The control unit may control the patch receiving unit or the substrate fixing unit such that the exposed surface of the patch faces the biological sample. The controlling of the patch receiving unit by the control unit may include directly or indirectly controlling the position and/or posture of the patch receiving unit. The controlling of the patch receiving unit by the control unit may include controlling the position and/or posture of the patch receiving unit by controlling the position and/or posture of a pressing head in contact with the patch receiving unit.

The test device may bring the patch in contact with the biological sample by controlling the relative position between the substrate and the patch. The test device may adjust the relative position of the patch to the biological sample such that a water film is formed in an area where the patch contacts the biological sample.

The control unit may control the patch receiving unit or the substrate fixing unit such that at least a portion of the exposed surface of the patch contacts the biological sample. The control unit may control the patch receiving unit or the substrate fixing unit such that at least a portion of the reagent contained in the patch is provided to the biological sample by bringing the patch to be in contact with the biological sample. The control unit may control the patch receiving unit or the substrate fixing unit such that the patch receiving unit or the substrate fixing unit moves along a guiding member that guides the movement of the patch receiving unit or the substrate fixing unit, so that the patch contacts the biological sample at a predetermined position.

The test device may space the patch from the biological sample. The test device may adjust the relative position of the patch with respect to the biological sample to space the patch apart from the biological sample. The control unit may control the patch receiving unit or the substrate fixing unit to space the patch apart from the biological sample.

### 4.3 Performing Diagnosis

Test results may be obtained by measuring the biological sample treated according to the above-described test process.

The obtaining of test results may be performed by optically observing the treated biological sample. The obtaining of test results may be performed by observing the biological sample under visible light. The obtaining of test results may be performed by observing the biological sample under fluorescent light.

The obtaining of test results may be performed by optically observing a target material included in the biological sample. The obtaining of test results may be performed by imaging an area in which the biological sample is distributed, and detecting the target material from the obtained image by using a machine-trained algorithm.

The obtaining of test results may be performed by measuring the biological sample by using an electrochemical method. The obtaining of test results may be performed by measuring the amount of the target material contained in the biological sample by using an electrochemical method.

Diagnosis of disease may be performed based on the test results obtained as described above. The performing of the diagnosis may include immunological diagnosis, genetic diagnosis, blood glucose measurement, etc.

### 4.4 Discussion

When a biological sample is tested by using a patch described according to the present specification as described above, an improved result may be obtained compared to a case using the conventional test method. According to an embodiment of the biological sample test method disclosed in the present specification, the amount of reagent used for the test may be reduced by delivering the reagent using the patch. By performing the test using the patch, the time required for the test may be shortened. In addition, by delivering a test reagent using the patch, the test accuracy of the biological sample may be improved. In addition, by delivering the reagent using the patch, the process of washing the biological sample may be omitted.

### 5. First Example of Test Method

### 5.1 Contact Equalization

When a patch disclosed in the present specification is brought into contact with a biological sample, air bubbles are formed between the biological sample and the patch, so that, in the corresponding area, the contact between the patch and the biological sample may be poor. Alternatively, the contact behavior between the patch and the specimen may vary according to the contact direction between the patch and the specimen and the state in which the specimen is smeared, and thus, the contact state between the patch and the specimen may be changed.

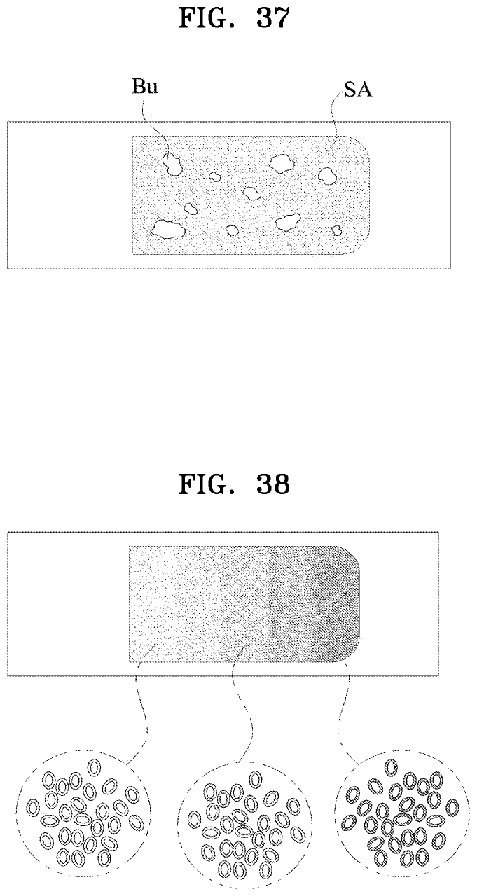

FIG. 37 illustrates an example of improperly staining when blood smeared on a substrate is stained. Referring to FIG. 37, in the case of staining the blood SA smeared on a substrate using a patch according to the present disclosure, air bubbles Bu may be formed between the patch and the specimen. The staining reagent may not be smoothly provided from the patch to the portion where the bubbles Bu are formed. Accordingly, the smeared blood SA may be non-uniformly stained as shown in FIG. 37.

FIG. 38 illustrates an example of improperly staining when blood smeared on a substrate is stained. Referring to FIG. 38, when the time maintained during the patch contacts one area is different from the time maintained during the patch contacts other area, a non-uniform staining result may be obtained as shown in FIG. 38. In other words, when the time when the patch contacts one area is longer than the time when the patch contacts another area, the one area may be exposed to the staining reagent longer, and the one area may be stained darker than the other area.

In order to solve the above-described problems, there is a need to more appropriately implement a contact method between the patch and the specimen. Hereinafter, in order to solve the above-described problems, a description will be given of a method of controlling the patch, which minimizes the generation of air bubbles and makes the patch uniformly contact the specimen.

In this regard, regarding the above-described test method of a biological sample, a test method of a biological sample in which a reagent is uniformly delivered from the patch to the biological sample by bringing the patch to be in contact with the biological sample disclosed in the present specification more uniformly, will be explained. In the following embodiments, the description of the test method provided above may be similarly applied unless described otherwise.

### 5.2 Method

The test method for a biological sample according to an embodiment of the present disclosure may include preparing a patch, allowing the patch to access the biological sample, and bring the patch to be in contact with the biological sample.

The preparing of the patch may include positioning the patch such that one surface of the patch faces the surface of a substrate by spacing the biological sample from the surface of the substrate, on which the biological sample is located, by a predetermined distance. The preparing of the patch may include positioning the patch such that one side of the patch is parallel to the surface of the substrate. The preparing of the patch may include positioning the patch such that one side of the patch is oblique to the surface of the substrate.

The allowing the patch to access the biological sample may include moving the patch toward the surface of the substrate while one surface of the patch is oblique to the surface of the substrate. The allowing the patch to access the biological sample may include moving the patch toward the surface of the substrate while the posture of the patch is changed such that one surface of the patch becomes oblique to the surface of the substrate.

The bringing the patch in contact with the biological sample may be bringing the patch to be in an oblique contact with the biological sample. In an embodiment, the bringing the patch in contact with the biological sample may include bringing the patch in contact with the biological sample such that one end of the patch contacts the biological sample before the other end thereof contacts the same. The bringing the patch in contact with the biological sample may include sequentially contacting the patch with the biological sample such that one area of the patch contacts the biological sample before the other area extending from the one area in a direction contacts the biological sample.

In this way, when areas of ( [symbol] ) the patch are sequentially in contact with the biological sample, the generation of air bubbles described with respect to FIG. 37 may be prevented. Since the patch is sequentially brought in contact with the biological sample, the air layer between the patch and the biological sample may be suppressed in a direction and thus the generation of air bubbles may be prevented.

Meanwhile, the above-described test method for biological samples may be applied to the case of staining a biological sample uniformly by using a patch containing a staining reagent. However, this is only an example of present disclosure, and the patch may be used to deliver a substance other than the staining reagent. Hereinafter, some embodiments of a method of controlling the patch posture to uniformly stain the biological sample will be described.

A method of controlling the posture of a patch according to the present disclosure may be a method of controlling the posture of a patch by using a gel-type patch which includes a staining reagent used to stain a specimen and a net structure forming micro-cavities for containing the staining reagent, and has a contact surface which is brought in contact with the specimen to deliver the staining reagent.

The patch posture control method may include bringing the patch to be in contact with the specimen, allowing the patch to access the patch toward the surface of the substrate, and bringing the patch to be in contact with the specimen.

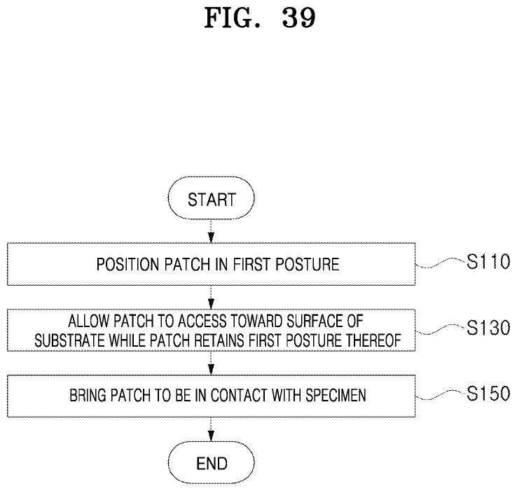

FIG. 39 illustrates a flowchart of a patch posture control method according to an embodiment of the present disclosure. Referring to FIG. 39, the patch posture control method includes positioning the patch in a first posture (S110), allowing the patch to access the surface of the substrate while the patch retains the first posture thereof (S130), and bringing the patch to be in contact with the specimen (S150).

The positioning the patch to be in a first posture (S110) may include positioning the patch to be a first posture in which the contact surface of the patch faces the surface of the substrate on which the specimen is placed and an end of the contact surface is closer to the surface of the substrate on which the specimen is placed than another end of the contact surface. In this regard, the allowing the patch to access the surface of the substrate may include allowing the patch to access the surface of the substrate while the patch retains the first posture as described above.

The positioning the patch to be in a first posture (S110) may include positioning the patch to be in a first posture in which the contact surface of the patch faces the surface of the substrate and the contact surface of the patch is parallel to the surface of the substrate. In this regard, the allowing the patch to access the surface of the substrate may include allowing the patch to access toward the surface of the substrate while the posture of the patch is changed from the first posture to an oblique posture in which the contact surface of the patch faces the surface of the substrate on which the specimen is placed and an end of the contact surface is closer to the surface of the substrate on which the specimen is placed than another end of the contact surface.

The bringing the patch to be in contact with the specimen (S150) may include sequentially bringing the first area and the second area of the patch to be in contact with the specimen.

Specifically, the second area is an area extending from the first area in a direction, and the bringing the patch in contact with the biological sample may include sequentially bringing the patch in contact with the biological sample in a direction.

The bringing the patch to be in contact with the specimen (S150) may include bringing the patch to be in contact with the specimen placed on the surface of the substrate so as to deliver the staining reagent to the specimen.

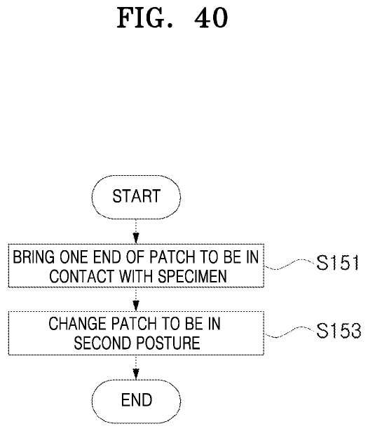

FIG. 40 illustrates contacting of a patch with a specimen in a patch posture control method according to an embodiment of the present disclosure. Referring to FIG. 40, the bringing the patch to be in contact with the specimen may include bringing an end of the patch to be in contact with the specimen while the patch is maintained to be in a first posture (S151), and changing the patch to have a second posture (S153).

The bringing an end of the patch to be in contact with the specimen (S151) may include bringing one end of the patch to be in contact with the specimen such that a water film is formed between the end in contact and the specimen.

The changing the posture of the patch to a second posture (S153) may include changing the posture of the patch such that while the patch is placed oblique to the surface of the substrate, the contact surface of the patch in contact with the specimen is to be parallel to the surface of the substrate. The changing the posture of the patch to a second posture may include changing the posture of the patch from the first posture to a second posture which is parallel to the surface of the substrate such that while the contact surface of the patch is fitted with the surface of the substrate, the contact area of the patch in contact with the specimen extends in a first direction.

The changing of the patch from the first posture to the second posture (S153) includes changing the posture of the patch such that when the contact surface of the patch sequentially accesses the surface of the substrate from the end to the other end of the contact surface, the water film is grown in the first direction.

The changing of the patch from the first posture to the second posture (S153) may further include changing the posture of the patch such that when the contact surface of the patch sequentially accesses the surface of the substrate from the end to the other end of the contact surface, a meniscus surface on one side of the water film moves in the first direction.

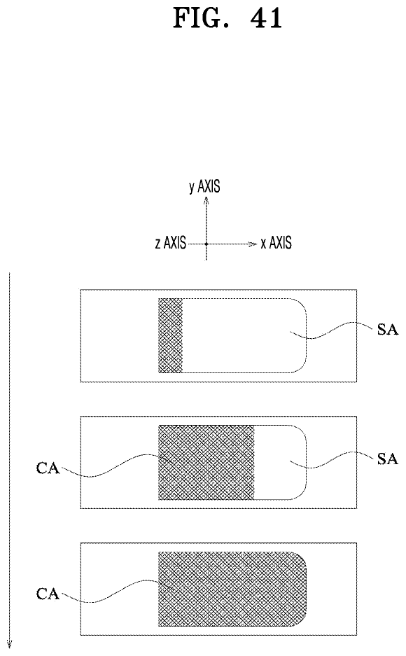

FIG. 41 illustrates an example in which as the patch is brought in contact with the specimen SA sequentially, the contact area CA between the patch and the specimen SA is widened. Referring to FIG. 41, the patch posture control method according to an embodiment of the present disclosure may include sequentially bringing the patch in contact with the specimen SA such that the contact area CA of the patch and the specimen SA is extended in the length direction (that is, the X-axis direction) of the substrate on which the specimen SA is located. The patch posture control method may include sequentially bringing the patch in contact with the specimen SA such that an edge of the water film formed between the patch and the specimen SA moves in the length direction of the substrate.

The widening of the contact area CA may be understood as widening the area in which the reagent contained in the patch is provided to the specimen SA.

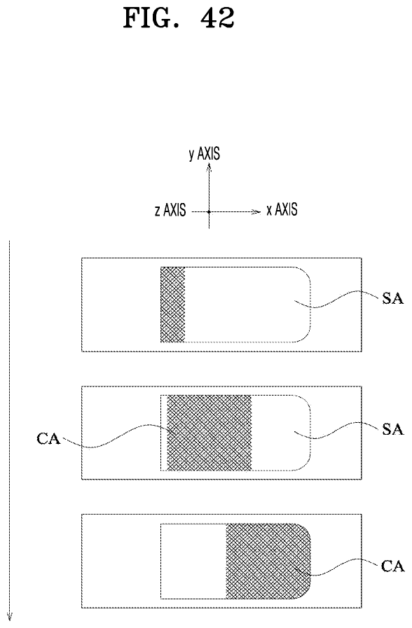

FIG. 41 illustrates an example in which as the patch is brought in contact with the specimen SA sequentially, the contact area CA between the patch and the specimen SA is moved in chronological order. FIG. 42 illustrates an example in which as the patch is brought in contact with the specimen SA sequentially, the contact area CA between the patch and the specimen SA is moved in chronological order. Referring to FIG. 42, a patch posture control method according to an embodiment of the present disclosure may include moving the contact area CA in the length direction (that is, the X-axis direction) of the substrate. In other words, the patch may sequentially contact the specimen SA in the first area and then the second area arranged in one direction, and the second area may contact the specimen SA while the first area is separated from the specimen SA.

Referring to FIG. 42, the patch posture control method may include moving an edge of the contact surface between the patch and the specimen SA in the length direction of the substrate. The contact area CA of the patch with respect to the specimen SA may maintain a certain area. Meanwhile, FIG. 42 illustrates an embodiment in which the edge of the contact surface CA moves in the length direction of the substrate. However, embodiments of the present disclosure are not limited thereto. In an embodiment, the edge may move in a width direction of the substrate.

Meanwhile, in the embodiments described above, the specimen may be blood smeared on the surface of the substrate. The specimen may be blood smeared on the surface of the substrate in one direction, that is, in the smearing direction. The smear direction may be a direction parallel to a first direction in which the first area and the second area of the patch are arranged. The direction in which the blood sample is smeared may be a direction perpendicular to the first direction in which the patch first area and the second area are arranged.

When the specimen is blood smeared on the substrate in one direction, the smear condition of the specimen may vary depending on the smearing direction. In this regard, the patch may be sequentially brought into contact with the specimen in the smearing direction so that the patch and the specimen uniformly contact each other in the smearing direction. In this regard, the bringing the patch into contact may further include bringing the patch to be in contact with the specimen such that the contact area is widened in the direction in which the blood is smeared so that the staining reagent is uniformly provided in the direction in which the blood is smeared.

When the specimen is blood spread on the substrate in one direction, constituents constituting the blood vary in density and volume, and thus the specimen may be smeared non-uniformly on the substrate. In this regard, in particular, in a direction perpendicular to the smearing direction (for example, the X-axis direction) in which the blood is smeared (e.g., the Y-axis direction), the components of the blood may be separated and smeared.

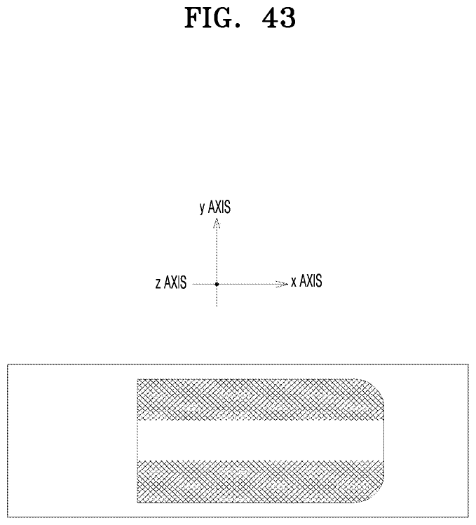

FIG. 43 illustrates an example of blood unevenly smeared on a substrate. Referring to FIG. 43, the blood may be smeared such that the components thereof are non-uniformly distributed in the width direction (that is, Y-axis direction) of the substrate. For example, as the blood sample is smeared in the X-axis direction, the blood sample is separated in the Y-axis direction, and a relatively large number of white blood cells may be distributed in an outer portion of the substrate, and a relatively large number of red blood cells may be distributed in inner portion thereof with respect to the area in which the white blood cells are distributed.

Due to the blood components separated as described above, a step may occur in the smeared blood. In this regard, the patch may be brought into contact with the specimen in a direction perpendicular to the smearing direction. In this case, the bringing the patch into contact may further include bringing the patch in contact with the specimen such that the contact area is perpendicular to the direction in which the blood is smeared so as to uniformly deliver the staining reagent and prevent the formation of air bubbles according to the step caused by components, with respect to the blood components smeared non-uniformly in the direction perpendicular to the direction in which the blood is smeared.

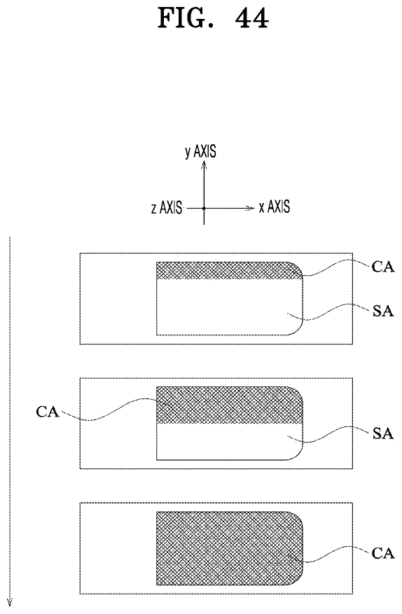

FIG. 44 illustrates an embodiment in which the contact area CA of the patch PA and the specimen SA is expanded, according to the patch control method according to an embodiment of the present disclosure. Referring to FIG. 44, the patch control method according to an embodiment of the present disclosure may include sequentially bringing the patch in contact with the specimen SA such that the contact area CA of the patch and the specimen SA is extended in the width direction (that is, the Y-axis direction) of the substrate on which the specimen SA is located.

The patch posture control method may further include spacing the patch from the surface of the substrate. The spacing the patch from the surface of the substrate may be performed by sequentially separating the patch from the specimen, as described later.

The spacing the patch from the surface of the substrate may include separating at least a portion of the contact surface from the specimen. The spacing the patch from the surface of the substrate may include changing the posture of the patch from the second posture to a third posture that is oblique to the surface of the substrate so that the contact area is reduced in the second direction. In this case, the second direction is a reverse direction of the first direction, and the third posture may be the same as the first posture.

The spacing the patch from the surface of the substrate may further include changing the posture of the patch such that the water film formed between the contact surface and the specimen is reduced in the second direction as at least a portion of the contact surface is separated from the specimen.

The spacing the patch from the surface of the substrate may further include changing the posture of the patch such that the patch is sequentially separated from the specimen from the other end to the end of the contact surface.

The second direction is parallel to the first direction, and the spacing the patch from the surface of the substrate may further include changing the posture of the patch such that the patch is sequentially separated from the specimen from the end to the other end of the contact surface.

The spacing the patch from the surface of the substrate may further include separating the contact surface from the specimen such that, with respect to the whole area of the specimen, the time during which the patch is brought into contact and continued to be in contact is constant, in order to uniformly deliver the staining reagent to the specimen.

The spacing the patch from the surface of the substrate may further include separating the contact area from the specimen such that the time during which the contact area extends from the end to the other end thereof is the same as the time during which the contact area is separated from the other end to the end thereof.

A test method for a biological sample according to another embodiment of the present disclosure may include sequentially bringing the patch containing the reagent to be in contact with the biological sample, and delivering the reagent to the biological sample.

The test method for the biological sample may include bringing the patch in contact with the biological sample such that the contact surface of the patch sequentially contacts the biological sample in one direction. The test method for the biological sample may include bringing the patch in oblique contact with the biological sample such that air bubbles are not formed between the patch and the biological sample. The test method for the biological sample may include bringing the patch in oblique contact with the biological sample such that air bubbles generated between the patch and the biological sample are pushed and removed in one direction. The test method for the biological sample may include bringing the patch in oblique contact with the biological sample such that the patch having a restoring force is deformed to fit the biological sample and placed close to the biological sample.

The delivering the reagent to the biological sample may be bringing the patch to be in contact with the biological sample to deliver the reagent contained in the patch to the biological sample. The delivering the reagent to the biological sample may include guiding at least some of the reagent to be released from the patch by pressing the patch against the biological sample with a certain force.

The test method for the specimen according to the above embodiment may be applied to a method of controlling the posture of the patch to deliver the staining reagent used for staining to the specimen by using a gel-type patch which includes the staining reagent and a net structure forming micro-cavities for containing the staining reagent, and has a contact surface, which is a surface to be brought into contact with the specimen.

The patch posture control method may include bring the patch to be in contact with the specimen. The bringing the patch to be in contact with the specimen may be bringing a side of the patch to be in contact with the specimen and sequentially bringing the patch to be in contact with the specimen from the side of the patch to another side thereof to prevent the formation of air bubbles between the contact surface and the specimen.

The patch posture control method may include delivering the staining reagent to the specimen through the contact surface of the patch.

The specimen may be a blood sample smeared on the surface of the substrate. The blood sample may be smeared on the surface of the substrate in one direction. In this regard, the patch posture control method may include sequentially bringing the contact surface to be in contact with the specimen in the direction in which the blood is smeared. The bringing the contact surface from the side to the other side thereof may include sequentially bringing the contact surface to be in contact in a direction perpendicular to the direction in which the blood is smeared.

The patch posture control method may further include separating the patch from the surface of the specimen. The separating the patch from the specimen may include sequentially separating the contact surface from the specimen from the side to the other side of the contact surface so as to prevent deformation of the specimen.

In this regard, the separating the patch from the specimen may be performed in consideration of the speed and timing at which the patch contacts the specimen to uniformly deliver the reagent to the whole area of the specimen. For example, the sequentially separating the contact surface from the specimen may include separating the contact surface from the specimen at a rate equal to a first speed at which the contact surface is brought into contact with the specimen from the side to the other side of the contact surface.

### 5.3 Device

According to one embodiment, the test of a biological sample using a patch disclosed in the present specification may be performed by a test device that includes a patch receiving unit receiving the patch and a substrate fixing unit fixing the substrate at a certain position. The description provided in connection with the performance of the test will be applied to the description about each of the patch receiving unit, the substrate fixing unit, and the test device including the patch receiving unit and the substrate fixing unit.

The patch receiving unit may have a shape corresponding to the patch. The patch receiving unit may move along a guiding member that guides the movement of the patch receiving unit such that the patch obliquely contacts the biological sample placed on the substrate at a predetermined position.

The patch may have a contact surface which is a curved surface with a length direction and a curve direction. In this regard, the bringing the patch to be in contact with the specimen may include bringing the patch to be in contact with the specimen such that the curved surface sequentially contacts the specimen. The bringing the patch to be in contact with the specimen may include sequentially bringing the patch to be in contact with the specimen in the curve direction.

The patch may have a cylindrical contact surface having a circumferential direction and a length direction. The bringing the patch to be in contact with the specimen may be sequentially bringing the contact surface of the patch in contact with the biological sample in the circumferential direction.

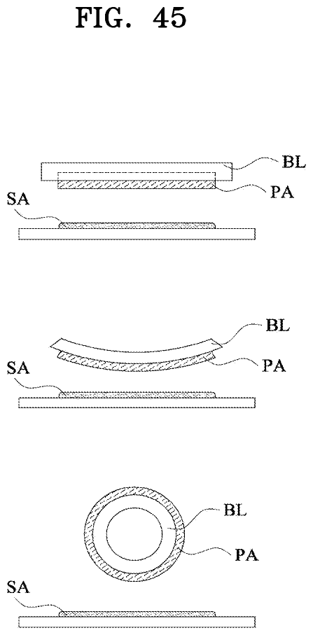

FIG. 45 is a side view of the patch PA, a patch receiving block BL, and a substrate PL according to some embodiments of the present disclosure.

Referring to (a) of FIG. 45A, the patch PA may have a contact surface which is flat. The patch PA may be fixed to the patch receiving block BL such that the contact surface protrudes or is exposed. Referring to (b) of FIG. 45B, the patch PA may have a contact surface which is curved. The patch PA may be fixed to the patch receiving block BL such that the contact surface protrudes or is exposed. Referring to (c) of FIG. 45, the patch PA may be provided in a cylindrical shape. The patch PA may be mounted and placed on a cylindrical patch receiving block BL

The substrate fixing unit may move along a guiding member that guides the movement of the substrate fixing unit such that the patch obliquely contacts the biological sample placed on the substrate at a predetermined position.

The test device may position the patch such that one side thereof is oblique with respect to the substrate. The test device may change the posture of the patch such that one side thereof is oblique with respect to the substrate.

The test device may contact the biological sample in a direction oblique with respect to the substrate. The test device may control the patch such that the exposed contact surface of the patch sequentially contacts the biological sample. The contact surface includes a first area and a second area extending from the first area in one direction, and the test device may sequentially bring the patch to be in contact with the biological sample in one direction such that the first area contacts the biological sample before the second area.

The test device may allow the patch to access obliquely to the biological sample. The test device may control the patch such that the contact surface contacts the biological sample while the patch is fitted with the surface of the substrate. The test device may bring the patch to be in oblique contact with the biological sample such that the contact surface of the patch is deformed corresponding to the biological sample.

The test device may further include a control unit. The description in connection with the test device provided above will be applied in the following embodiments.

The control unit may control the patch receiving unit and/or the substrate fixing unit such that one side of the patch is positioned oblique to the surface of the substrate. The control unit may control the patch receiving unit and/or the substrate fixing unit such that one side of the patch becomes positioned oblique to the surface of the substrate.

The controlling of the patch receiving unit and/or the substrate fixing unit by the control unit may include direct or indirect control of the patch receiving unit and/or the substrate fixing unit.

The control unit may control the patch receiving unit or the substrate fixing unit such that the patch accesses oblique to the surface of the substrate. The control unit may control the patch receiving unit or the substrate fixing unit such that the patch contacts oblique to the surface of the substrate to form a water film between the patch and the biological sample.

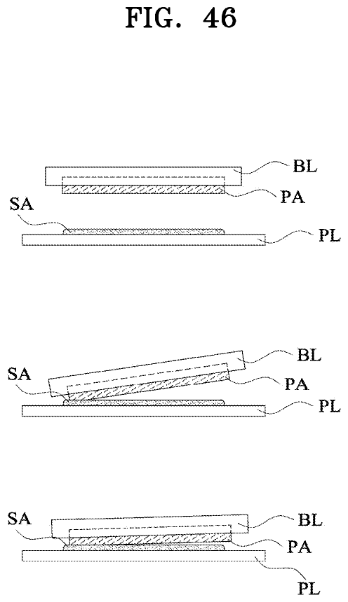

FIG. 46 schematically illustrates a process of delivering a reagent to a biological sample by using a test device according to an embodiment of the present disclosure. Referring to FIG. 46, the patch may be provided in a plate shape having a length, a width and a height. Referring to FIG. 46, in the operation of the test device, a patch receiving block BL having a flat contact surface and receiving the patch is lowered from one side thereof and thus, the patch contacts the biological sample from the side of the patch receiving block BL. The patch receiving block BL may raise and lower along a guiding member providing the path of the patch receiving block BL such that the patch PA may contact the biological sample.

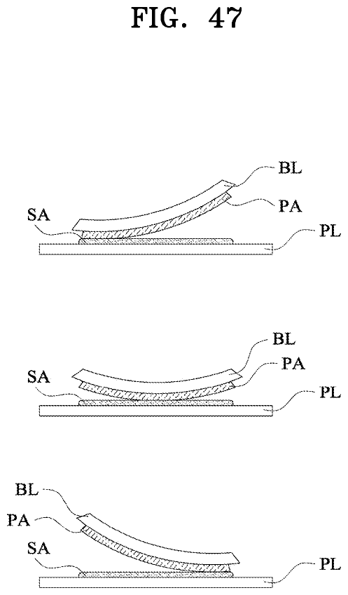

FIG. 47 schematically illustrates a process of delivering a reagent to a biological sample by using a test device according to an embodiment of the present disclosure. Referring to FIG. 47, the patch may be provided as a curved plate. Referring to FIG. 47, in the operation of the test device, the patch receiving block BL having a curved contact surface and containing the patch is lowered from one side thereof so that the patch contacts the biological sample from the side thereof. The patch receiving block BL may raise and lower or move in a curve motion along a guiding member providing the path of the patch receiving block BL such that the patch may contact the biological sample.

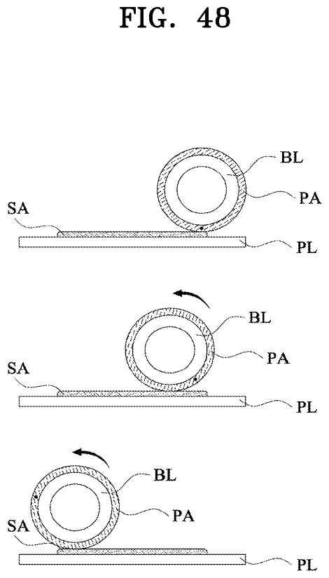

FIG. 48 schematically illustrates a process of delivering a reagent to a biological sample by using a test device according to an embodiment of the present disclosure. Referring to FIG. 48, in the operation of the test device, the patch receiving block BL containing a cylindrical patch is brought into contact with the biological sample from one side to the other side of the patch receiving block BL while rolling so that the reagent is delivered to the reagent from the side to the other side of the patch receiving block BL. The patch receiving block BL may raise and lower or move back and forth along a guiding member providing the path of the patch receiving block BL such that the patch PA may contact the biological sample.

Meanwhile, in FIGS. 46 to 48, the patch is described based on the case where the patch receiving unit is a patch receiving block BL, but the disclosure disclosed in the present specification is not limited thereto. The patch receiving unit described in FIGS. 46 to 48 may be a patch receiving member whose position and/or posture is not directly controlled by a power source.

According to one embodiment of the present disclosure, provided is a patch posture control device in which a staining reagent used for staining a specimen is contained, the patch posture control device for staining the specimen using a patch having a contact surface that is brought into contact with the specimen to deliver the staining reagent to the specimen. The description of the test device described above may be applied to the patch posture control device.

According to one embodiment of the present disclosure, the patch posture control device may include a substrate fixing unit that fixes a substrate on which the specimen is placed, a patch receiving block that receives the patch such that at least a portion of the contact surface of the patch is exposed, and a contact surface for controlling the relative position of the patch receiving block with respect to the substrate.

The control unit may control the patch receiving block to position the patch to be in a first posture at which the contact surface faces the surface of the substrate on which the specimen is placed and an end of the contact surface is closer to the surface of the substrate on which the specimen is placed than another end of the contact surface. The control unit may control the patch receiving block such that while the patch is maintained at the first posture, the patch is allowed to access the surface of the substrate and the patch is brought into contact with the specimen to deliver the staining reagent to the specimen.

The control unit may control the patch receiving block such that, at the first posture, an end of the patch contacts the specimen placed on the surface of the substrate, and when the posture of the patch is changed from the first posture to a second posture at which the contact surface is parallel to the surface of the substrate, the contact surface of the patch is fitted with the surface of the substrate and the contact area of the surface of the substrate in contact with the specimen may extend in a first direction.

The controlling the patch receiving block to bring an end of the patch at the first posture to be in contact with the specimen placed on the surface of the substrate, may include bringing the end of the patch to be in contact with the specimen to form a water film between the contact end and the specimen.

The control unit may control the posture of the patch receiving block such that the posture of the patch is changed to the second posture.

The controlling of the patch receiving block to change the posture of the patch from the first posture to the second posture by the control unit may include controlling the patch receiving block such that when the posture of the patch is changed to the second posture, the contact surface is sequentially allowed to access the surface of the substrate from the end of the contact surface to the other end thereof to grow the water film formed between the end and the specimen in the first direction.

The control unit may control the patch receiving block to space the patch apart from the surface of the substrate. In this regard, the controlling the patch receiving block by the control unit may include controlling the posture of the patch receiving block such that the posture of the patch is changed from the second posture to a third posture at which the patch is placed oblique to the surface of the substrate and thus at least a portion of the contact surface is separated from the specimen and the contact area is reduced in a second direction.

In the above embodiment, the second direction is the reverse direction of the first direction, and the third posture may be implemented in the same way as the first posture. In this regard, the control unit may control the patch receiving block such that the patch is sequentially separated from the specimen from the other end to the end of the contact surface.

The second direction may be the same direction as the first direction. In this regard, the control unit may control the patch receiving block such that the patch is sequentially separated from the specimen from the end of the contact surface to the other end thereof.

The controlling, by the control unit, the posture of the patch receiving block to make the patch to be spaced apart from the surface of the substrate may include controlling the patch receiving block to make the time during which the patch contacts the whole area of the patch and retains the contact state thereof to be constant so as to uniformly deliver the staining reagent to the whole area of the specimen.

According to one embodiment of the present disclosure, provided is a patch posture control device including a substrate on which the specimen is located, a patch receiving block receiving the patch such that at least a portion of the contact surface is exposed, and a control unit controlling the relative position of the patch receiving block with respect to the substrate, wherein the control unit controls the posture of the patch receiving block to bring an side of the patch to be in contact with the specimen.

The control unit may control the posture of the patch receiving block such that, to prevent the formation of air bubbles between the contact surface and the specimen, the contact surface is sequentially brought into contact the specimen from one side to another side of the specimen, and through the contact surface of the patch, the staining reagent is delivered to the specimen.

The control unit may control the posture of the patch receiving block such that the contact surface of the patch is sequentially separated from the side to the other side of the specimen so that the staining reagent is uniformly delivered to the area where the specimen is distributed.

The patch receiving block may have a deformed edge to prevent the edge from being caught by the substrate or the specimen in the process of bringing the patch in oblique contact to the surface of the substrate. The patch receiving block may have at least one rounded edge so that the edge of the patch is not caught by the substrate or the specimen.

The control unit may control the posture of the patch receiving block such that, to minimize the friction between the patch receiving block and the substrate or the specimen when the patch contacts the specimen sequentially, the patch receiving block is allowed to access the substrate from the rounded edge thereof.

The controlling, by the control unit, the posture of the patch receiving block to bring the contact surface into contact with the specimen sequentially may further include controlling the posture of the patch receiving block such that the contact surface contacts the specimen from a side to another side of the patch at a first speed.

The controlling, by the control unit, the posture of the patch receiving block to separate the contact surface from the side to the other side of the patch, may further include separating the contact surface from the specimen at the first speed to uniformly deliver the staining reagent to the specimen.

The controlling, by the control unit, the posture of the patch receiving block to separate the contact surface from the side to the other side of the patch, may further include controlling the posture of the patch receiving block such that the time during which the patch is in contact and retains the contact state thereof is constant with respect to the whole area of the specimen to uniformly deliver the staining reagent to the specimen.

According to an embodiment of the present disclosure, the patch posture control device may include a kit receiving unit including a kit which includes at least one of a substrate fixing unit fixing the substrate on which the specimen is located and a patch receiving member which receives the patch such that at least a portion of the contact surface is exposed.

The description of the patch receiving unit described above may be applied to the patch receiving member.

The configuration and operation of the patch receiving member and/or the pressing head may be implemented in a similar manner to the patch receiving block described above. In other words, in the present embodiment, the patch receiving member and/or the pressing head may perform a function similar to that of the patch receiving block in the embodiments described above. The patch receiving member may be sequentially allowed to access the specimen from one side to another side of the specimen by being pressed by the pressing head. The contact area of the specimen and the patch received in the patch receiving member may extend in one direction.

The patch receiving member may be obliquely pressed by the pressing head as the pressing head accesses obliquely to the surface of the substrate or the upper surface of the patch receiving member. The patch receiving member may access obliquely to the surface of the substrate as being obliquely pressed by the pressing head. The patch receiving member may sequentially contact the specimen positioned on the surface of the substrate from one side of the specimen as being pressed by the pressing head.

According to one embodiment of the present disclosure, the patch posture control device may include the substrate fixing unit, the kit receiving unit, and a control unit for controlling the substrate fixing unit and/or the kit receiving unit.

The control unit may control the position of the patch receiving member relative to the substrate.

The control unit may locate the patch to be in a first posture at which an end of the contact surface is closer to the surface of the substrate on which the specimen is placed than another end of the contact surface while the patch faces the surface of the substrate on which the specimen is placed, allow the patch to access the surface of the substrate while maintaining the patch to be at the first posture, and bring the patch to be in contact with the specimen placed on the surface of the substrate to deliver the staining reagent to the specimen.

The control unit may further control the position of the patch receiving member relative to the surface of the substrate such that, at the first posture, an end of the patch contacts the specimen placed on the surface of the substrate, and when the posture of the patch is changed from the first posture to a second posture at which the contact surface is parallel to the surface of the substrate, the contact surface of the patch is fitted with the surface of the substrate and the contact area of the surface of the substrate in contact with the specimen may extend in a first direction.

The controlling, by the control unit, the patch receiving member to bring an end of the patch at the first posture to be in contact with the specimen placed on the surface of the substrate, may include bringing the end of the patch to be in contact with the specimen to form a water film between the end in contact and the specimen.

The changing the patch from the first posture to the second posture by the control unit may include changing the posture of the patch to the second posture so as to grow the water film formed between the end of the contact surface and the specimen in the first direction by allowing the contact surface to sequentially access the surface of the substrate from the end to the other end of the contact surface.

The control unit may control the posture of the patch receiving member to space the patch apart from the surface of the substrate. The controlling, by the control unit, the posture of the patch receiving member to space the patch from the surface of the substrate may further include controlling the posture of the patch receiving member such that the patch is changed from the second posture to a third posture in which the patch is oblique to the surface of the substrate so that at least a portion of the contact surface is separated from the specimen and the contact area is reduced in the second direction.

The second direction is the reverse direction of the first direction, the third posture is the same as the first posture, and the control unit may control the posture of the patch receiving member such that the patch is sequentially separated from the specimen from the other end to the end of the contact surface.

The second direction is a direction parallel to the first direction, and the control unit may control the posture of the patch receiving member to sequentially separate the patch from the specimen from the end to the other end of the contact surface.

The controlling, by the control unit, the posture of the patch receiving member to make the patch to be spaced apart from the surface of the substrate may further include controlling the posture of the patch receiving member to make the time, during which the patch contacts the whole area of the patch and retains the contact state thereof, to be constant so as to uniformly deliver the staining reagent to the whole area of the specimen.

Meanwhile, according to one embodiment of the present disclosure, the patch posture control device may further include a pressing head that contacts the patch receiving member and provides a pressing force on the patch receiving block.

The controlling, by the control unit, the position of the patch receiving member relative to the substrate, may include controlling, by the control unit, the relative position of the patch receiving member which contacts the pressing head by controlling the position of the patch receiving member by the pressing head.

Hereinafter, the operation of the patch posture control device in the case where the patch posture control device includes the pressing head, will be described.

According to one embodiment of the present disclosure, the patch posture control device may include the substrate fixing unit, the kit receiving unit, the pressing head, and a control unit for controlling the substrate fixing unit, the kit receiving unit, and/or the pressing head.

The control unit may perform various operations to be described later by using the patch receiving member and/or the pressing head. The control unit may control the patch receiving member or the pressing head. In this regard, controlling the patch receiving member by the control unit may include indirectly controlling, by using the pressing head, the patch receiving member which is pressed by the pressing head.

The control unit may control the patch receiving member or the pressing head to position the patch to be in the first posture at which the contact surface faces the surface of the substrate on which the specimen is placed and the end of the contact surface is closer to the surface of the substrate on which the specimen is placed than the other end of the contact surface. The control unit may control the pressing head or the patch receiving member to position the patch to be with the first posture by controlling the position and/posture of the pressing head.

The control unit may control the patch receiving member or the pressing head to gradually extend the contact area as the patch changes from the first posture to the second posture, which is a posture parallel to the surface of the substrate. The control unit may control the pressing head contacting the patch receiving member to extend the contact area in the first direction as the position and/or posture of the patch receiving member is changed and the posture of the patch is changed to the second posture.

The control unit may control the patch receiving member or the pressing head to form a water film between the specimen and the contact area of the patch. The control unit may control the patch receiving member pressed by the pressing head by controlling the pressing head to form the water film.

The control unit may control the patch receiving member or the pressing head to change the posture of the patch from the first posture to the second posture. The control unit may control the patch receiving member or the pressing head to change the posture of the patch to the second posture to grow the water film in the first direction.

The control unit may control the patch receiving member or the pressing head to space the patch apart from the surface of the substrate. The control unit may control the pressing head or the patch receiving member to change the posture of the patch from the second posture to the third posture which is oblique to the surface of the substrate, resulting in the decrease in the contact area in the second direction.

The second direction may be the reverse direction of the first direction. The third posture may be implemented as an inclined posture similar to the first posture. The control unit may control the patch receiving member or the pressing head to sequentially separate the patch from the specimen from the other end of the contact surface.

The second direction may be the same direction as the first direction. The control unit may control the patch receiving member or the pressing head to sequentially separate the patch from the specimen from the end of the contact surface.

The controlling, by the control unit, the patch receiving member or the pressing head to make the patch to be spaced apart from the surface of the substrate may further include controlling the patch receiving member or the pressing head to make the time, during which the patch contacts the whole area of the patch and retains the contact state thereof, to be constant so as to uniformly deliver the staining reagent to the whole area of the specimen.

### 6. Second Example of Test Method

### 6.1 Stabilization of Separation

In the case of performing a test of a biological sample using the patch, negative pressure acts on the biological sample in the process of separating the patch from the biological sample, which may damage or deform the biological sample and negatively affect the test result. In order to prevent such deformation of the sample, it is required to implement a more appropriate test method.

In this regard, a method of testing a biological sample in which a reagent is delivered to a biological sample using a patch disclosed in the present specification, and damage or deformation of the biological sample is minimized in the process of delivering the reagent to the biological sample, will be described. In the following embodiments, the description of the test method provided above may be similarly applied unless described otherwise.

### 6.2 Method

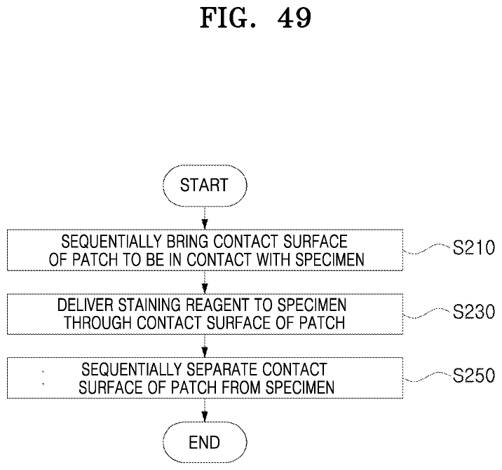

FIG. 49 illustrates a flowchart of a test method according to an embodiment of the present disclosure. Referring to FIG. 49, in the test method according to an embodiment of the present disclosure may include sequentially bringing a contact surface of the patch to be in contact with a specimen (S210), delivering a staining reagent to the specimen through the contact surface of the patch (S230), and sequentially separating the contact surface of the patch from the specimen (S250).

The bringing a contact surface of the patch to be in contact with a specimen (S210) and delivering a staining reagent to the specimen through the contact surface of the patch (S230) may be implemented in the similar manner to the embodiments described above. FIG. 42 illustrates an embodiment in which the contact surface of the patch is sequentially, obliquely brought into contact with the specimen. However, embodiments of the present disclosure are not limited thereto. In some embodiments, even when the contact surface is brought into contact with the specimen while the contact surface is parallel to the specimen, the contact surface may be sequentially separated from the specimen.

The separating the patch from the biological sample (S250) may refer to spacing the contact surface of the patch in contact with the biological sample away from the biological sample. The spacing of the patch away from the biological sample may include returning the patch to be in the position thereof at which the patch has been before contacting the biological sample. The spacing of the patch from the biological sample may refer to changing the relative position of the patch with respect to the substrate on which the biological sample is located. The spacing the patch from the biological sample may include separating the patch from the biological sample to break the connection between the patch and the biological sample.

The spacing the patch from the biological sample may include spacing, from the biological sample, the patch in contact with the biological sample sequentially from one side to another side of the patch. The spacing the patch from the biological sample may include spacing the patch from the biological sample obliquely to prevent deformation of the biological sample. The spacing the patch from the biological sample may include spacing the patch from the biological sample sequentially from one side thereof to minimize the occurrence of negative pressure in the biological sample.

According to one embodiment of the test method, the patch may be spaced from the biological sample to reduce the contact area of the patch in contact with the biological sample. In this regard, the contact area of the patch in contact with the biological sample may be reduced in one direction. The spacing the patch may be performed by spacing the contact surface away from the biological sample sequentially in one direction to reduce the contact area in one direction.

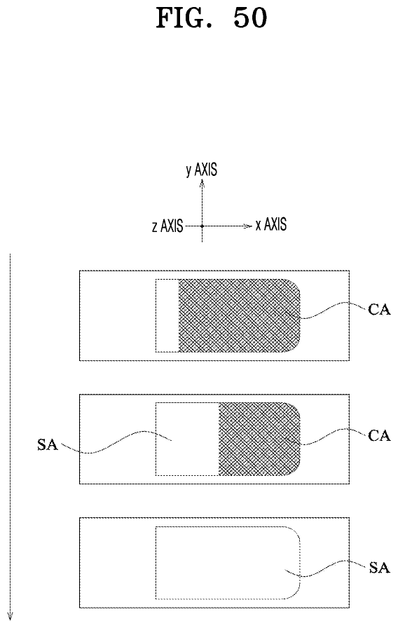

FIG. 50 illustrates an embodiment in which the contact area CA between the patch and the biological sample SA is reduced, according to the patch control method according to an embodiment of the present disclosure. Referring to FIG. 50, the test method according to an embodiment of the present disclosure may include sequentially separating the patch from the specimen SA to reduce the contact area CA of the patch with respect to the biological sample SA in the length direction (that is, an X-axis direction) of the substrate on which the specimen is located.

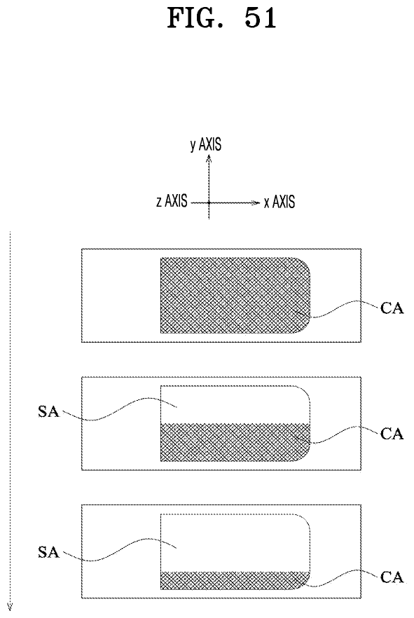

FIG. 51 illustrates another embodiment in which the contact area CA between the patch and the biological sample SA is reduced, according to the test method according to an embodiment of the present disclosure. Referring to FIG. 51, the test method may include sequentially separating the patch from the specimen to reduce the contact area CA between the patch and the specimen in the width direction of the substrate on which the specimen is located.

The spacing the patch may include spacing the patch such that an edge of the contact area which is in contact with the biological sample SA, is moved in one direction.

The spacing of the patch may be performed by sequentially spacing the contact area CA from the biological sample SA in one direction so that an edge of the contact area moves in one direction.

According to one embodiment, the areas constituting the contact area CA may be sequentially separated from the biological sample SA. The contact area CA may include a first area and a second area located in one direction with respect to the first area, and the test method may include obliquely spacing the patch from the biological sample SA to separate the first area from the biological sample SA and then the second area from the biological sample SA. In this regard, the first area may be separated from the specimen while the second area is in contact with the specimen.

According to an embodiment, the spacing of the patch from the biological sample SA refers to spacing of a portion of the contact area CA from the biological sample SA while another portion of the contact area CA is in contact with the biological sample SA. In other words, according to an embodiment of the test method for the biological sample SA, a portion of the patch may be spaced from the biological sample SA such that the contact area moves in one direction as shown in FIG. 42.

Meanwhile, according to an embodiment of the present disclosure, the biological sample may be blood smeared on the substrate. In this regard, considering the direction in which the blood is smeared on the substrate, the direction in which the patch is sequentially brought into contact with the biological sample.

In the case in which blood is smeared on the substrate in a first direction (for example, an X-axis direction), spacing the patch from the biological sample may involve controlling the patch to reduce the contact surface in the first direction. The patch may be spaced in the same direction as the direction in which the blood is smeared to uniformly deliver the reagent to the whole area of the smeared blood. In this regard, the test method may further include, prior to the spacing from the patch from the biological sample, sequentially bring the contact surface of the patch into contact with the biological sample in the first direction or a second direction perpendicular to the first direction.

The spacing the patch from the biological sample may include controlling the patch to reduce the contact surface in the second direction perpendicular to the first direction (for example, a Y-axis direction). By spacing the patch in a direction perpendicular to the direction in which the blood is smeared, the influence of the steps formed due to separated components which may occur when the blood is smeared non-uniformly as illustrated in FIG. 40 may be minimized. The direction in which the patch is spaced from the biological sample may be parallel or perpendicular to the direction in which the patch is sequentially brought in contact with the biological sample.

According to an embodiment of the present disclosure, the test method described above may be implemented as a patch posture control method to stain the specimen.

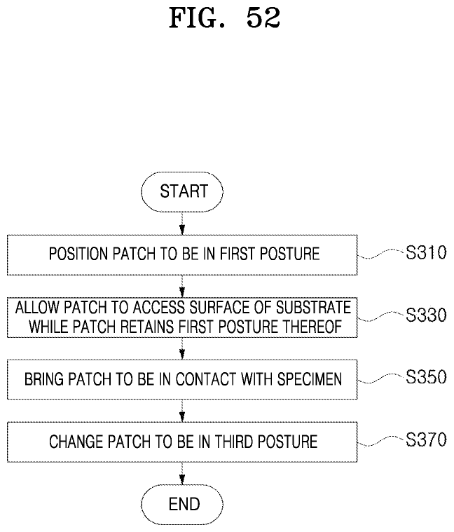

FIG. 52 illustrates a flowchart of a patch posture control method according to an embodiment of the present disclosure. Referring to FIG. 52, the patch posture control method includes placing the patch in a first posture on one side of the surface of the substrate (S310), allowing the patch to access the surface of the substrate (S330), bringing the patch to be in contact with the specimen (S350), and changing the patch to be in a third posture (S370).

The positioning the patch to be in the first posture (S310) is to position the patch to be on one side of the surface of the substrate in a first posture at which the contact surface of the patch faces the surface of the substrate on which the specimen is placed.

The positioning the patch to be in a first posture (S310) may include positioning the patch to be a first posture in which the contact surface of the patch faces the surface of the substrate on which the specimen is placed and an end of the contact surface is closer to the surface of the substrate on which the specimen is placed than another end of the contact surface.

The allowing the patch to access the surface of the substrate may include allowing the patch to access the surface of the substrate while the patch retains the first posture as described above.

The bringing the patch to be in contact with the specimen (S350) may include changing the posture of the patch from the first posture to the second posture in which at least a portion of the contact surface contacts the specimen to bring the patch to be in contact with the specimen, thereby delivering the staining reagent to the specimen.

The bringing the patch to be in contact with the specimen (S350) may further include changing the patch from the first posture to the second posture by bringing the end of the contact surface to be in contact with the specimen before the other end thereof contacts the specimen so that the contact area in which the contact surface contacts the specimen extends and the patch sequentially contacts the specimen, in the direction from the end to the other end of the contact surface. In one or more embodiments, the patch may be changed to the second posture to extend the contact area from the other end in the end thereof and to sequentially bringing the patch to be in contact with the specimen.

The bringing the patch to be in contact with the specimen (S350) may further include the bringing the patch to be in contact with the specimen to form a water film between the contact surface and the specimen.

The changing the patch to the third posture (S370) may include spacing the patch from the surface of the substrate to separate the end of the contact surface from the surface of the substrate than the other end of the contact surface, so that the patch is in the third posture in which the patch is oblique to the surface of the substrate.

The changing the patch to the third posture (S370) may include changing the patch from the second posture to the third posture to reduce the contact area in which the contact surface contacts the specimen in one direction.

The changing the patch to the third posture (S370) may further include changing the posture of the patch to reduce the water film in one direction as the contact surface is sequentially separated from the specimen from the end to the other end of the contact surface.

The changing the patch to the third posture may further include changing the posture of the patch to the third posture such that, as the contact surface is sequentially spaced from the surface of the substrate from the end to the other end thereof, the meniscus surface of the water film is moved toward the other end of the contact surface.

The spacing the patch from the surface of the substrate, may be implemented by moving the patch upwards away from the substrate located under the patch.

The spacing the patch from the surface of the substrate may further include separating the contact surface from the specimen such that, with respect to the whole area of the specimen, the time during which the patch is brought into contact and continued to be in contact is constant, in order to uniformly deliver the staining reagent to the specimen.

In the patch posture control method, the specimen may be blood smeared on the surface of the substrate. The blood may be smeared in one direction.

In this case, the bringing the patch to be in contact with the specimen may include bringing the patch to be in contact with the specimen such that the contact area between contact surface contacts the specimen extends in a direction perpendicular to the direction in which the blood is smeared on the substrate so as to uniformly deliver the staining reagent to the blood components smeared non-uniformly in the direction perpendicular to the direction in which the blood is smeared.

In this regard, the bringing the patch to be in contact with the specimen may further include bringing the patch to be in contact with the specimen such that the contact area in which the contact surface contacts the specimen extends in the direction in which the blood is smeared so that the staining reagent is uniformly provided in the direction in which the blood is smeared.

### 6.3 Device

According to one embodiment, the test of a biological sample using a patch disclosed in the present specification may be performed by a test device that includes a patch receiving unit and a substrate fixing unit. Each of the patch receiving unit, the substrate fixing unit, and the test device including the patch receiving unit and the substrate fixing unit may be understood in the similar manner to those described above.

The test device may separate the patch that contacts the biological sample from the biological sample and deliver a reagent to the biological sample.

The separating the patch from the biological sample by using the test device may be performed in the reverse order of the contact of the patch described with reference to FIGS. 46 to 48. The separating of the patch from the biological sample by the test device may be performed in the reverse order of those shown in FIGS. 46 to 48. Referring to FIGS. 46 to 48, the separating the patch from the biological sample by the test device may include separating the patch from the biological sample to sequentially space the contact surface of the patch away from one side to another side of the patch.

According to the present specification, a patch control device to deliver the staining reagent to the specimen by using a gel-type patch which includes a staining reagent used for staining a specimen and a net structure forming micro-cavities for containing the staining reagent, and has, as a surface to be brought into contact with the specimen, the contact surface, is provided as an example of the test device.

The patch control device may include a substrate fixing unit for fixing the substrate on which the specimen is placed, a kit including at least one patch receiving member which receives the patch such that at least a portion of the contact surface is exposed to the outside, and a control unit to control the position of the patch receiving member with respect to the substrate.

The control unit may control the posture of the patch receiving member such that one side of the patch is brought in contact with the specimen, the contact surface is sequentially brought in contact with the specimen from one side to the other of the patch to prevent the formation of air bubbles between the contact surface and the specimen, the staining reagent is delivered to the specimen through the contact surface of the patch, and the contact surface of the patch is sequentially separated from one side to the other side of the patch to uniformly deliver the staining reagent to the area in which the specimen is distributed.

The patch receiving member has a rounded edge, and the control unit controls the posture of the patch receiving member such that the patch receiving member is allowed to access the substrate from the rounded edge of the patch receiving member so as to minimize the friction between the patch receiving member and the substrate or the specimen when the patch is sequentially brought into the specimen.

The controlling, by the control unit, the posture of the patch receiving member to bring the contact surface into contact with the specimen sequentially may further include controlling the posture of the patch receiving member such that the contact surface contacts the specimen from a side to another side of the patch at a first speed.

The controlling, by the control unit, the posture of the patch receiving member to separate the contact surface from the side to the other side of the patch, may further include separating the contact surface from the specimen at the first speed to uniformly deliver the staining reagent to the specimen.

The controlling, by the control unit, the posture of the patch receiving member to separate the contact surface from the side to the other side of the patch, may further include controlling the posture of the patch receiving member such that the time during which the patch is in contact and retains the contact state thereof is constant with respect to the whole area of the specimen to uniformly deliver the staining reagent to the specimen.

The patch posture control device may further include a pressing head which is in contact with the patch receiving member to provide a pressing force to the patch receiving member.

In this regard, the controlling, by the control unit, the position of the patch receiving member relative to the substrate, may include controlling, by the control unit, the relative position of the patch receiving member which contacts the pressing head by controlling the position of the patch receiving member by the pressing head.

According to one embodiment of the present disclosure, the patch posture control device may include the substrate fixing unit, the kit receiving unit, the pressing head, and a control unit for controlling the substrate fixing unit, the kit receiving unit, and/or the pressing head.

The control unit may control the patch posture control device such that the contact surface sequentially contacts the specimen in one direction to deliver the staining reagent to the specimen through the contact surface. The control unit may control the substrate fixing unit, the patch receiving member, or the pressing head such that one side of the patch is brought in contact with the specimen, the contact surface is sequentially brought in contact with the specimen from one side to the other of the patch to prevent the formation of air bubbles between the contact surface and the specimen, the staining reagent is delivered to the specimen through the contact surface of the patch, and the contact surface of the patch is sequentially separated from one side to the other side of the patch to uniformly deliver the staining reagent to the area in which the specimen is distributed.

The controlling, by the control unit, the posture of the patch receiving member or the pressing head to bring the contact surface into contact with the specimen sequentially may further include controlling the posture of the patch receiving member or the pressing head such that the contact surface contacts the specimen from a side to another side of the patch at a first speed.

The controlling, by the control unit, the posture of the patch receiving member or the pressing head to separate the contact surface from the side to the other side of the patch may further include controlling the patch receiving member or the pressing head such that the contact surface is separated from the specimen to separate the contact surface from the specimen at the first speed to uniformly deliver the staining reagent to the specimen.

The controlling, by the control unit, the posture of the patch receiving member to separate the contact surface from the side to the other side of the patch, may further include controlling the posture of the patch receiving member such that the time during which the patch is in contact and retains the contact state thereof is constant with respect to the whole area of the specimen to uniformly deliver the staining reagent to the specimen.

According to the present specification, a patch control device to deliver the staining reagent to the specimen by using a gel-type patch which includes a staining reagent used for staining a specimen and a net structure forming micro-cavities for containing the staining reagent, and has a contact surface to be brought into contact with the specimen to allow the staining reagent to be delivered therethrough, may be provided as an example of the test device.

The patch control device may include a substrate fixing unit that fixes a substrate on which the specimen is placed, a patch receiving block that receives the patch such that at least a portion of the contact surface is exposed to the outside. The operation of the patch control device may be understood in a similar manner as described above in previous embodiments.

The patch control device may space the contact surface of the patch from the specimen sequentially from one side to the other of the patch. The patch control device may space the patch from the specimen such that the contact area of the patch and the specimen is reduced to one side of the patch. The patch control device may space the patch from the specimen such that an edge of the contact area of the patch and the specimen is moved in one direction.

The patch control device may further contain a control unit that controls the position of the patch receiving block relative to the substrate.

The control unit may control the posture of the patch receiving block such that the patch is located in a first posture on one side of the surface of the substrate while the contact surface of the patch faces the surface of the substrate on which the specimen is placed, the patch accesses the surface of the substrate while retaining the first posture thereof, the posture of the patch is changed from the first posture to a second posture in which at least a portion of the contact surface contacts the specimen so that the patch contacts the specimen placed on the surface of the substrate, thereby delivering the staining reagent to the specimen, separating an end of the contact surface from the surface of the substrate, and then another end of the contact surface therefrom so that the patch is changed to a third posture in which the patch is positioned oblique to the surface of the substrate.

The controlling, by the control unit, the patch to change the posture of the patch receiving block to the third posture may further include changing the patch from the second posture to the third posture to reduce the contact area in which the contact surface contacts the specimen in one direction.

The controlling, by the control unit, the posture of the patch receiving block to bring the patch to be in contact with the specimen may include controlling the posture of the patch receiving block such that a water film is formed between the contact surface and the specimen.

The controlling, by the control unit, the posture of the patch receiving block to change the patch to be in the third posture may further include controlling the posture of the patch receiving block such that, as the contact surface is sequentially separated from the specimen from the end to the other end of the contact surface, the water film formed between the contact surface and the specimen is reduced in one direction.

The controlling, by the control unit, the posture of the patch receiving block to change the patch to be in the first posture may include controlling, by the control unit, the posture of the patch receiving block such that while the contact surface of the patch faces the surface of the substrate on which the specimen is placed, the patch is positioned in the first posture, that is, an oblique posture in which an end of the contact surface is closer to the surface of the substrate on which the specimen is placed than another surface of the contact surface.

The controlling, by the control unit, the posture of the patch receiving block to bring the patch to be in contact with the specimen may further include controlling the posture of the patch receiving block such that the end of the contact surface is in contact with the specimen before the other end thereof, and as the patch is changed from the first posture to the second posture, the contact area in which the contact surface contacts the specimen extends from the end to the other end of the contact surface to bring the patch to be in contact with the specimen sequentially.

The controlling, by the control unit, the posture of the patch receiving block to space the patch from the surface of the substrate may further include controlling the posture of the patch receiving block such that the time taken for the contact area to extend from the end to the other end thereof is the same as the time taken for the contact area to be reduced from the end to the other end thereof to uniformly deliver the staining reagent to the whole area of the specimen.

The controlling, by the control unit, the posture of the patch receiving block to bring the patch to be in contact with the specimen may further include controlling the posture of the patch receiving block such that the patch is changed from the first posture to the second posture to bring the other end of the contact surface to be in contact with the specimen before the end thereof, and thus, the contact area in which the contact surface contacts the specimen extends from the other end to the end of the contact surface to bring the patch to be in contact with the specimen sequentially.

According to the present specification, a patch posture control device to deliver the staining reagent to the specimen by using a gel-type patch which includes a staining reagent used for staining a specimen and a net structure forming micro-cavities for containing the staining reagent, and has a contact surface to be brought into contact with the specimen to allow the staining reagent to be delivered therethrough, may be provided as another example of the test device.

The patch posture control device may include a substrate fixing unit for fixing the substrate, a kit including at least one patch receiving member to receive the patch such that at least a portion of the contact surface of the patch is exposed to the outside, and a control unit to control the position of the patch receiving member with respect to the substrate. The descriptions about the test device, the patch, and the patch control device may be applied in a similar manner to the patch posture control device.

The control unit may control the posture of the patch receiving member such that the patch is located in a first posture on one side of the surface of the substrate while the contact surface of the patch faces the surface of the substrate on which the specimen is placed, the patch accesses the surface of the substrate while retaining the first posture thereof, the posture of the patch is changed from the first posture to a second posture in which at least a portion of the contact surface contacts the specimen so that the patch contacts the specimen placed on the surface of the substrate, thereby delivering the staining reagent to the specimen, separating an end of the contact surface from the surface of the substrate, and then another end of the contact surface therefrom so that the patch is changed to a third posture in which the patch is positioned oblique to the surface of the substrate.

The controlling, by the control unit, the patch to change the posture of the patch receiving member to the third posture may further include changing the patch from the second posture to the third posture to reduce the contact area in which the contact surface contacts the specimen in one direction.

The controlling, by the control unit, the posture of the patch receiving member to bring the patch to be in contact with the specimen may include controlling the posture of the patch receiving member such that a water film is formed between the contact surface and the specimen.

The controlling, by the control unit, the posture of the patch receiving member to change the patch to be in the third posture may further include controlling the posture of the patch receiving member such that, as the contact surface is sequentially separated from the specimen from the end to the other end of the contact surface, the water film formed between the contact surface and the specimen is reduced in one direction.

The controlling, by the control unit, the posture of the patch receiving member to change the patch to be in the first posture may include controlling, by the control unit, the posture of the patch receiving member such that while the contact surface of the patch faces the surface of the substrate on which the specimen is placed, the patch is positioned in the first posture, that is, an oblique posture in which an end of the contact surface is closer to the surface of the substrate on which the specimen is placed than another surface of the contact surface.

The controlling, by the control unit, the posture of the patch receiving member to bring the patch to be in contact with the specimen may further include controlling the posture of the patch receiving member such that the end of the contact surface is in contact with the specimen before the other end thereof, and as the patch is changed from the first posture to the second posture, the contact area in which the contact surface contacts the specimen extends from the end to the other end of the contact surface to bring the patch to be in contact with the specimen sequentially.

The controlling, by the control unit, the posture of the patch receiving member to space the patch from the surface of the substrate may further include controlling the posture of the patch receiving member such that the time taken for the contact area to extend from the end to the other end thereof is the same as the time taken for the contact area to be reduced from the end to the other end thereof to uniformly deliver the staining reagent to the whole area of the specimen.

The controlling, by the control unit, the posture of the patch receiving member to bring the patch to be in contact with the specimen may further include controlling the posture of the patch receiving member such that the patch is changed from the first posture to the second posture to bring the other end of the contact surface to be in contact with the specimen before the end thereof, and thus, the contact area in which the contact surface contacts the specimen extends from the other end to the end of the contact surface to bring the patch to be in contact with the specimen sequentially.

The patch posture control device may further include a pressing head which is in contact with the patch receiving member to provide a pressing force to the patch receiving member.

The controlling, by the control unit, the position of the patch receiving member relative to the substrate, may include controlling, by the control unit, the relative position of the patch receiving member which contacts the pressing head by controlling the position of the patch receiving member by the pressing head.

According to one embodiment of the present disclosure, the patch posture control device may include the substrate fixing unit, the kit receiving unit, the pressing head, and a control unit for controlling the substrate fixing unit, the kit receiving unit, and/or the pressing head.

The control unit may control the pressing head or the patch receiving member to perform the operation of the patch posture control device according to the embodiments described above.

### 7. Third Example of Test Method

### 7.1 Identification of Accuracy of Test Results

In order to efficiently deliver a sample, the patch may be pressed with a certain pressure to release at least a portion of a reagent contained in the patch. In this regard, when the patch is separated from the sample in a conventional manner, some of the released reagent may remain in the sample. Since the reagent remaining in the sample may interfere with the test of the reagent, a separate process is required to reabsorb excess reagent.

Hereinafter, some embodiments of a method of removing residue from the specimen using the patch will be described.

### 7.2 Method

In performing a test of a biological sample using a patch disclosed in the present specification, a process of reabsorbing a substance may be added. The reabsorbing the substance may be used to reabsorb the substance released from the patch or to remove the substance from the biological sample.

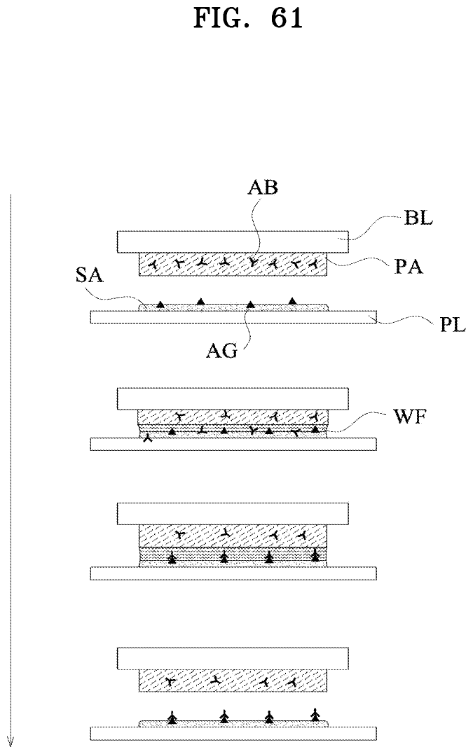

FIG. 61 schematically illustrates an embodiment of a test method according to a present disclosure in the order of time. Referring to FIG. 61, a test method according to an embodiment of the present disclosure may include detecting an antigen AG from a blood sample SA smeared on a substrate PL by using a patch PA containing an antibody AB.

Referring to FIG. 61, the test method may include preparing a substrate on which blood is smeared and a patch containing an antibody AB. The patch PA may be provided being fixed to the patch receiving block BL. The blood may be provided being fixed to the substrate.

Referring to FIG. 61, the test method may include providing the antibody AB to the blood sample SA by bringing the patch PA containing the antibody AB to be in contact with the blood sample SA. The providing the antibody AB may be performed by bringing the patch PA to be in contact with the blood sample SA so that the antibody AB may move to the blood sample SA through a water film formed in a contact area between the patch PA and the blood sample SA. The providing the antibody AB to the blood sample SA by bringing the patch PA to be in contact with the blood sample SA may include pressing the patch PA toward the substrate to release the antibody AB from at least a portion of the patch PA.

Referring to FIG. 61, the test method may further include reabsorbing a substance. The reabsorbing the substance may include maintaining the connection between the patch PA and the blood sample SA for a certain period of time while the substance is not released from the patch PA.

Referring to FIG. 61, the test method may further include raising the patch PA by a certain distance so that the patch PA moves away from the substrate while the water film is maintained (the third picture in FIG. 61). The raising the patch PA by a certain distance may be performed by reducing the force applied to the patch receiving block BL. The test method may further include raising the patch PA by a certain distance so that due to the decrease in the pressure applied to the patch PA, a reagent, for example, the antibody AB released through the water film is reabsorbed by the patch PA.

According to the present disclosure, when the patch PA is not immediately spaced from the sample and undergoes the process of reabsorbing, the excess release of the reagent from the patch PA to the sample may be prevented. In detail, the patch PA may prevent a portion of the reagent which is released from the patch PA and does not react with the target material included in the sample, from remaining in the sample. For example, due to the reabsorbing, a portion of the antibody AB which is released from the patch PA and is not bound to the antigen AG may be removed from the blood sample SA, thereby increasing the accuracy of the test.

Meanwhile, although not shown in FIG. 61, the bringing the patch PA to be in contact with the blood sample SA may be performed in a similar manner to the descriptions provide in connection with first example. The bringing the patch PA to be in contact with the blood sample SA may be implemented by sequentially bringing the patch PA to be in contact with the blood sample SA from one side of the patch PA, as described in the first example of the test method provided above.

Referring to FIG. 61, the test method may further include spacing the patch PA from the blood sample SA. The test method may further include spacing the patch PA from the blood sample SA such that a portion of the antibody AB which is released from the patch PA and is not bound to the antigen AG included in the blood sample SA, is separated, together with at least a portion of the patch PA, from the blood sample SA.

Meanwhile, although not shown in FIG. 61, the spacing the patch PA from the blood sample SA may be performed in a similar manner to the descriptions provide in connection with the second example. The spacing the patch PA from the blood sample SA may be implemented by sequentially spacing the patch PA from the blood sample SA from one side of the patch PA, as described in the second example of the test method provided above.

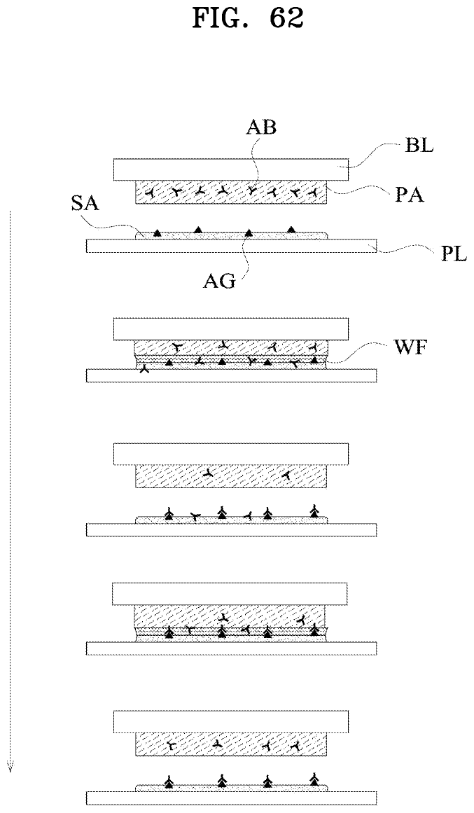

FIG. 62 schematically illustrates an embodiment of a test method according to a present disclosure in the order of time. Referring to FIG. 62, the test method according to an embodiment of the present disclosure may be implemented by adding, to the test method described in FIG. 61, the process of reabsorbing the residue from the blood sample SA by re-contacting after the spacing of the patch PA. The test method described in connection with FIG. 62 will be described based on the features distinguishable from the test method described in FIG. 61.

Referring to FIG. 62, like the test method described in connection with FIG. 61, the test method may include preparing a substrate and a patch PA, bringing the patch PA to be in contact with a blood sample SA located on the substrate to deliver the antibody AB to the blood sample SA, reabsorbing the residual antibody AB by using the patch PA, and spacing the patch PA from the blood sample SA.

Referring to FIG. 62, unlike the test method shown in FIG. 61, the test method may further include, after the bringing the patch PA to be in contact with the blood sample SA to deliver the antibody AB to the blood sample SA and before the reabsorbing the residual antibody AB by using the patch PA, spacing the patch PA from the blood sample SA to separate the patch PA therefrom.

In other words, the reabsorbing the residual antibody AB by using the patch PA may further include spacing the patch PA from the blood sample SA to separate the patch PA from the blood sample SA and then re-bringing the patch PA to be in contact with the blood sample SA to reabsorb foreign substances or the residual.

Referring to FIG. 62, the re-bringing the patch PA to be in contact with the blood sample SA may be implemented by lowering the patch PA toward the substrate by applying a smaller pressure than that applied to the patch PA when the patch PA is brought into contact with the blood sample SA to release the antibody AB from the patch PA.

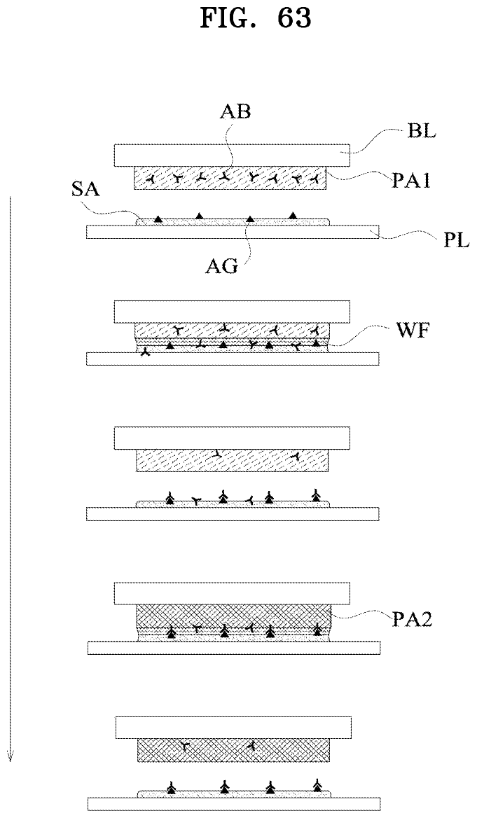

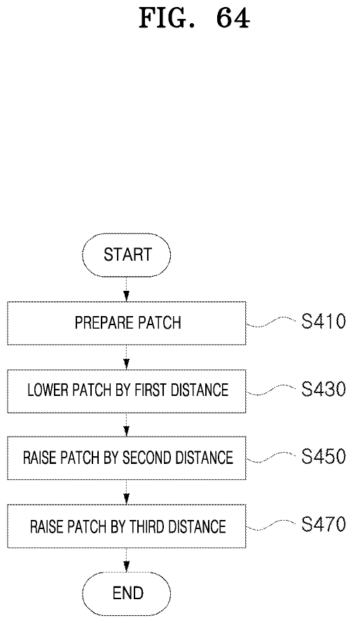

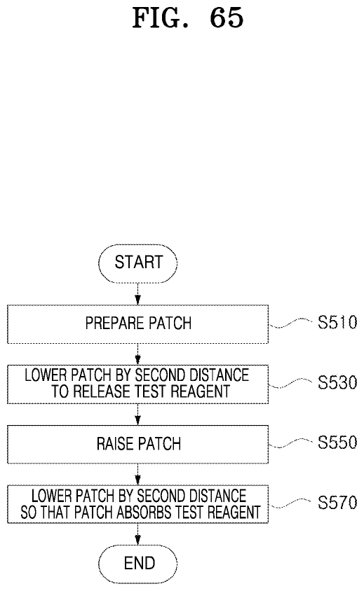

FIG. 63 schematically illustrates an embodiment of a test method according to a present disclosure in the order of time. Referring to FIG. 63, the test method according to an embodiment of the present disclosure may be implemented by adding, to the test method described in connection with FIG. 61, using an absorbing patch and reabsorbing the residual antibody AB using the absorbing patch. The test method described in connection with FIG. 63 will be described based on the features distinguishable from the test method described in FIG. 61.

Referring to FIG. 63, like the test method described in connection with FIG. 61, the test method may include preparing a substrate and a patch, bringing the patch to be in contact with a blood sample SA located on the substrate to deliver the antibody AB to the blood sample SA, and spacing the patch from the blood sample SA.

Referring to FIG. 63, the test method may further include using a separate absorbing patch for containing an absorbing reagent. The absorbing patch may be brought into contact with the blood sample SA and then separated therefrom to absorb and remove reaction residues and impurities from the plate PL.

The absorbing patch may contain a water-soluble or fat-soluble absorbing reagent to absorb reaction residues or foreign substances located on the specimen.

The absorbing patch may contain a washing solution. For example, the absorbing patch may contain TBS or PBS supplemented with tween-20. However, the absorbing patch is not limited to the embodiments described above. The absorbing patch is distinguished from a patch that delivers a reagent, and may be selected from various types of patches that contain a smaller amount of target reagent than that the patch to deliver the reagent.

Meanwhile, in the embodiments described in connection with FIGS. 61 to 63, in testing a biological sample, a patch containing the antibody AB is used to detect the antibody AB included in the biological sample. However, the present disclosure described in the present specification is not limited thereto. The test method according to the present disclosure may be applied to various cases of performing a test of a sample using one or more of the aforementioned patches.

On the other hand, the present disclosure provides a test method for a specimen, in which a gel-type patch including a net structure forming micro-cavities which contain a test reagent that is used for the test by reacting with a target material included in the specimen, is used to minimize the residual of a substance that does not react with the target in the specimen. Hereinafter, some embodiments of the test method will be described with reference to FIGS. 64 to 67.

FIG. 64 is a flowchart illustrating an embodiment of a test method according to the present disclosure. Referring to FIG. 64, a test method according to an embodiment of the present disclosure may include preparing a patch (S410), lowering the patch by a first distance (S430), and raising the patch by a second distance (S450) and raising the present disclosure by a third distance (S470).

The preparing the patch (S410) may be implemented by preparing a patch containing the test reagent above the specimen. The preparing the patch (S410) may be implemented by using a method that is similar to those described in connection with the previous embodiments.

The lowering the patch by a first distance (S430) may include lowering the patch toward the specimen by a predetermined first distance by applying a predetermined intensity of pressure to the patch to release at least a portion of the test reagent from the patch.

The lowering the patch by a first distance (S430) may further include lowering the patch such that the patch is brought into contact with the specimen to form a water film in the contact area.

The lowering the patch may include lowering the patch obliquely so that one side of the patch is brought into contact with the specimen before another side of the patch. In this regard, the lowering the patch obliquely may be implemented by using a method that is similar to that described in the first example.

The raising the patch by a second distance (S450) may include raising the patch by a predetermined second distance in a direction away from the specimen so that at least a portion of the pressure acting on the patch is reduced to allow the patch to absorb at least a portion of the test reagent that is provided to the specimen and does not react with the target material.

In the raising the patch by a second distance (S450), the patch is connected to the specimen via the water film, and the water film may include at least a portion of the test reagent that is provided to the specimen and does not react with the target material.

The raising the patch by the second distance (S450) may further include raising the patch by the second distance while the water film formed between the patch and the specimen is maintained.

The raising the patch by a third distance (S470) may include raising the patch by a predetermined third distance in a direction away from the specimen so that the patch is spaced from the specimen.

The raising the patch by a third distance may be obliquely raising the patch away from the specimen so that one side of the patch is spaced from the specimen before the other side of the patch to prevent deformation of the specimen.

The target material and the test reagent may perform specific reactions. The target material is an antigen, and the test reagent may include an antibody that reacts with the antigen. The test reagent may contain a staining reagent that labels the target material so that the target material is optically detectable.

FIG. 65 is a flowchart illustrating an embodiment of a test method according to the present disclosure. Referring to FIG. 65, a test method according to an embodiment of the present disclosure may include preparing the patch (S510), lowering the patch by a first distance to release the test reagent from the patch (S530), raising the patch (S550), and lowering the patch by a second distance to allow the patch to absorb the test reagent (S570).

The preparing the patch (S510) may include preparing a patch containing the test reagent above the specimen. The preparing the patch (S510) may be implemented by using a method that is similar to those described in connection with the previous embodiments.

The lowering the patch by a first distance to release the test reagent from the patch (S530) may include lowering the patch toward the specimen by a predetermined first distance to apply a predetermined first pressure to the patch to release at least a portion of the test reagent from the patch.

The raising the patch (S550) may be raising the patch in a direction away from the specimen so that the patch is spaced from the specimen. The raising the patch may be obliquely raising the patch away from the specimen so that one side of the patch is spaced from the specimen before the other side of the patch to prevent deformation of the specimen.

The lowering the patch by a second distance to allow the patch to absorb the test reagent (S570) may further include lowering the patch toward the second distance by a predetermined second distance so that a second pressure, which is weaker than the first pressure, is applied to the patch so that the patch absorbs at least a portion of the test reagent that is provided to the specimen and does not react with the target material.

In the lowering the patch by a second distance to allow the patch to absorb the test reagent (S570), the patch is connected to the specimen via the water film, and the water film includes at least a portion of the test reagent that is provided to the specimen and does not react with the target material.

The lowering the patch by a second distance to allow the patch to absorb the test reagent (S570) may further include lowering the patch by a second distance to connect the patch to the specimen via the water film while the test reagent is not released from the patch for a certain period of time.

The test method may further include, after the lowering the patch by the second distance (S570), raising the patch to separate, from the specimen, at least a portion of the test reagent that is provided to the specimen and does not react with the target material, together with the patch. in this regard, the lowering the patch may include lowering the patch obliquely so that one side of the patch is brought into contact with the specimen before another side of the patch.

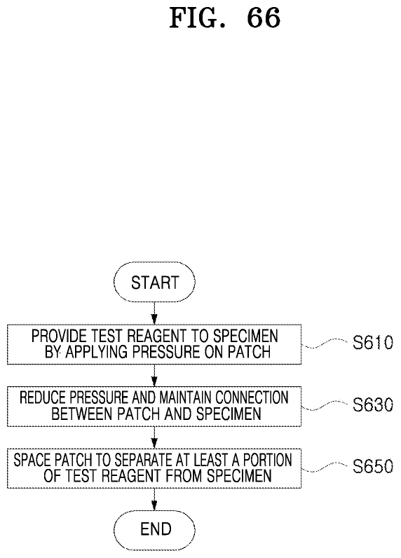

FIG. 66 is a flowchart illustrating an embodiment of a test method according to the present disclosure. Referring to FIG. 66, a test method according to an embodiment of the present disclosure may include, applying pressure to the patch to provide a test reagent to the specimen (S610), reducing the pressure acting on the patch to maintain the state in which the patch is connected with the specimen (S630) and spacing the patch from the specimen so that at least a portion of the test method from the specimen (S650).

The applying pressure to the patch to provide the test reagent to the specimen (S610) may be applying pressure to the patch to release the test reagent from the patch to the specimen, thereby providing the test reagent to the specimen. The providing the test reagent to the specimen (S610) may be performed with a method that is similar to those described in connection with the previous embodiments.

The reducing the pressure acting on the patch to keep the patch connected to the specimen (S630) is achieved by reducing the pressure applied to the patch to maintain the connection between the patch and the specimen via the water film while the water film includes at least a portion of the test reagent that is provided to the specimen and does not react with the target material. The state in which the patch is connected to the specimen refers to the state in which the test reagent is not released from the patch.

The keeping the patch connected to the specimen (S630) may further include raising the patch a certain distance away from the specimen so that the pressure applied to the patch is reduced.

The spacing the patch from the specimen to separate at least a portion of the test reagent from the specimen (S650) may include spacing the patch from the specimen to separate, from the specimen, at least a portion of the test reagent which is included in the water film and does not react with the target material.

The spacing the patch to separate at least a portion of the test reagent from the specimen (S650) may further include raising the patch obliquely away from the specimen to space the patch away from the specimen so that one side of the patch is spaced from the specimen than another side thereof.

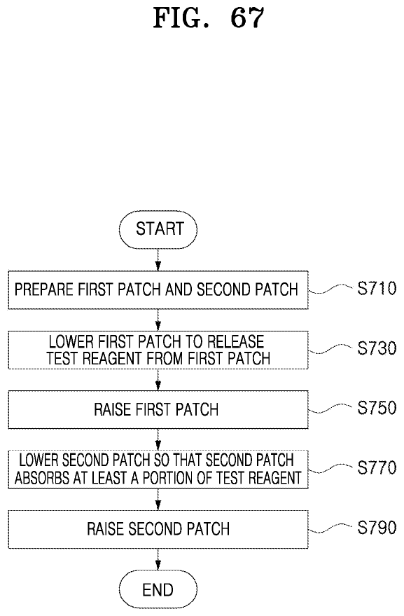

FIG. 67 is a flowchart illustrating an embodiment of a test method according to the present disclosure. Referring to FIG. 67, a test method according to an embodiment of the present disclosure may include preparing a first patch and a second patch (S710), lowering the first patch to release a test reagent from the first patch (S730), raising the first patch (S750), lowering the second patch to allow the patch to absorb the test reagent (S770), and raising the second patch (S790).

The preparing a first patch and a second patch (S710) may be preparing a first patch containing the test reagent and a second patch absorbing the test reagent. The test reagent contained in the first patch may be the same as described in connection with the previous embodiments. The second patch that absorbs the reagent may contain the absorbing reagent. The absorbing reagent may be the same as described in the previous embodiments.

The lowering the first patch to release the test reagent from the first patch (S530) may be implemented by lowering the first patch toward the specimen to apply a predetermined pressure to the first patch to release at least a portion of the test reagent from the first patch. The lowering the first patch may further include lowering the first patch obliquely so that one side of the first patch is brought into contact with the specimen before another side of the first patch.

The raising the first patch (S750) may be selectively performed after the lowering the first patch toward the specimen. The raising the first patch (S750) may be raising the first patch in a direction away from the specimen so that the first patch is spaced from the specimen.

The lowering a second patch (S770) may be implemented by lowering the second patch toward the specimen such that the second patch is connected to the specimen to form a water film between the second patch and the specimen and through the water film, at least a portion of the test reagent that is provided to the specimen and does not react with the target material is absorbed by the second patch.

The raising the second patch (S790) may be raising the second patch in a direction away from the specimen such that the second patch is separated from the specimen. The raising the second patch (S790) may further include spacing the second patch from the specimen to separate, from the specimen, the second patch which absorbs the test reagent, so as to prevent the test reagent which is provided to the specimen and does not react with the target material from remaining in the specimen.

The raising the second patch may be obliquely raising the second patch away from the specimen so that one side of the second patch is spaced from the specimen before the other side of the second patch to prevent deformation of the specimen.

### 7.3 Device

The embodiments of the third example of the test method may be implemented by using, for example, the test method disclosed in the present specification. The operation of the test device may be understood in a similar manner as described above in previous embodiments.

According to an embodiment of the present disclosure, provided is a test device for a specimen, in which a gel-type patch including a net structure forming micro-cavities which contain a test reagent that is used for the test by reacting with a target material included in the specimen, is used to minimize the residual of a substance that does not react with the target in the specimen.

The test device may include a substrate fixing unit that fixes a substrate on which a specimen is placed, a patch receiving block that receives the patch such that at least a portion of the contact surface is exposed to the outside.

The test device may prepare a patch for containing the test reagent above the specimen.

The test device may lower the patch toward the specimen such that a predetermined pressure acts on the patch to release at least a portion of the test reagent from the patch.

The test device may raise the patch by a predetermined second distance in a direction away from the specimen so that at least a portion of the pressure acting on the patch is reduced to allow the patch to absorb at least a portion of the test reagent that is provided to the specimen and does not react with the target material.

The test device may raise the patch by a predetermined third distance away from the specimen such that the patch is spaced from the specimen.

The test device may further include a control unit. The control unit may control a patch receiving block that receives the patch and/or a substrate fixing unit to which the substrate is fixed.

The control unit may control the position of the patch receiving block or the substrate fixing unit to place the patch containing the test reagent above the specimen.

The control unit may lower the patch toward the specimen by a predetermined first distance such that a predetermined pressure acts on the patch to release at least a portion of the test reagent from the patch. The control unit may control the position of the patch receiving block such that the patch lowers toward the specimen by a predetermined first distance.

The control unit may raise the patch by a predetermined second distance in a direction away from the specimen so that at least a portion of the pressure acting on the patch is reduced to allow the patch to absorb at least a portion of the test reagent that is provided to the specimen and does not react with the target material. The control unit may control the position of the patch receiving block such that the patch is raised in a direction away from the specimen by a predetermined second distance.

The control unit may raise the patch by a predetermined third distance away from the specimen such that the patch is spaced from the specimen. The control unit may control the position of the patch receiving block to space the patch apart from the surface of the specimen.

According to another embodiment of the present disclosure, the test device may prepare a first patch containing the test reagent and a second patch absorbing the test reagent.

The test device may lower the first patch toward the specimen such that a predetermined pressure acts on the first patch to release at least a portion of the test reagent from the first patch.

The test device may be implemented by lowering the second patch toward the specimen such that the second patch is connected to the specimen to form a water film between the second patch and the specimen and through the water film, at least a portion of the test reagent that is provided to the specimen and does not react with the target material is absorbed by the second patch.

The test device may raise the second patch in a direction away from the specimen such that the second patch is separated from the specimen.

The test device may further include a control unit. To perform the operations described above, the test device may use the control unit to control the position of the patch receiving block receiving the patch or the substrate fixing unit on which the substrate is fixed.

### 8. Examples of Test Device

### 8.1 Test Device

The test method and the patch control method described above may be performed by using a test device or a patch control device, respectively. The test device or the like may commonly include a patch receiving block or a patch receiving member, as a patch receiving unit that receives a patch, and a substrate fixing unit that fixes a substrate on which a sample is fixed.

Hereinafter, some embodiments of a test device or a patch posture control device for implementing embodiments of the test method will be described. The respective embodiments described below do not necessarily have to be individually implemented, and it is obvious that one or more embodiments may be combined.

### 8.2 First Embodiment: One Rotating Shaft

A test device according to one embodiment of the present disclosure may include the patch receiving block and an elastic member. The patch receiving block may receive the patch and directly access a substrate. The test device according to the present embodiment may test, for example, a patch by using the patch receiving block, wherein the patch receiving block is manufactured to be rotatable about one rotating shaft.

The patch receiving block may include a patch receiving block which is arranged to be rotatable about a rotating shaft parallel to a Y-axis direction. The rotating shaft may be located to pass through the center of the patch receiving block. The rotating shaft may be located on the outside of the patch receiving block. The rotating shaft may be located on one side of the patch receiving block.

The elastic member may be provided to be in contact with one side of the posture of the patch receiving block depending on the posture of the patch receiving block. The elastic member may be connected to one side of the patch receiving block. The elastic member may apply a tensile or compressive force on one side of the patch receiving block to keep the posture of the patch receiving block oblique to the substrate on which the sample is located.

The elastic member may be a metallic or non-metallic spring. The elastic member may be a compression spring or a tension spring. The elastic member may be a torsion spring or a leaf spring. The elastic member may be made of an elastic material such as rubber or silicone.

The test device may further include a support member which is connected to the patch receiving block and on which one side of the elastic member is fixed. The support member may provide up and down movements of the patch receiving block.

The test device may further include a guiding member that provides the movement path of the patch receiving block. The guiding member may provide a movement path of the support member to guide the patch to move to a predetermined location.

The test device may further include a motor. The motor may change the position of the patch receiving block in the Z-axis direction. The motor may change the position of the support member in the Z-axis direction. As the motor, a linear step motor (or stepping motor or stepper motor) may be used.

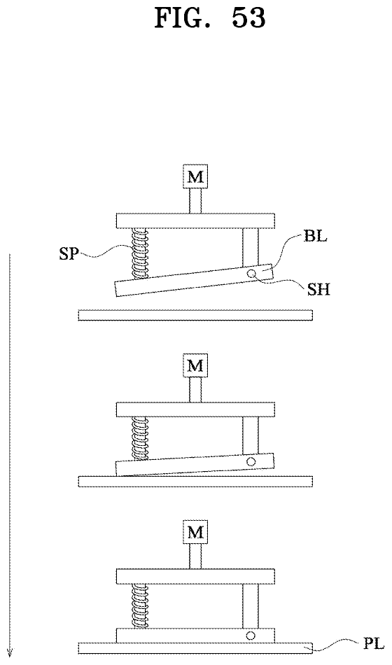

FIG. 53 illustrates an operation of a test device according to an embodiment of the present disclosure in the order of time. Referring to FIG. 53, a test of a biological sample located on a substrate PL may be performed by using a test device including a motor M, a spring SP, a patch receiving block BL, and a support member SP. Although not shown in FIG. 53, the patch receiving block may include a patch receiving unit in which the patch is located, and the patch receiving block may receive the patch such that one surface of the patch protrudes or is exposed.

Referring to FIG. 53, the test device may prepare the patch receiving block BL to be oblique to the substrate PL. The patch receiving block BL may be prepared with one side thereof closer to the substrate PL than the other side by the elastic force of the spring SP.

Referring to FIG. 53, the test device may drive the motor to lower the patch receiving block BL. The test device may drive the motor to lower a rotating shaft SH connected to the patch receiving block BL. The test device may drive the motor to allow the patch receiving block to access the substrate PL.

As the test device drives the motor, the patch receiving block BL lowers toward the substrate so that one side thereof may be in contact with the substrate PL. As the test device drives the motor, the rotating shaft SH lowers and one side of the patch receiving block may be in contact with the substrate directly or indirectly. In this regard, as the patch receiving block lowers, a patch of which one surface protrudes or is exposed downward may be in contact with the sample located on the substrate PL.

As one side of the patch receiving block is in contact with the substrate, a force may be applied to the one side thereof in a direction perpendicular to the substrate. Due to the force applied to the one side of the patch receiving block in the direction perpendicular to the substrate, the patch receiving block may rotate so as to be aligned with the substrate around the rotation axis SH. In this regard, as the patch receiving block rotates, the patch may sequentially be in contact with the sample from one side thereof as illustrated in FIG. 53.

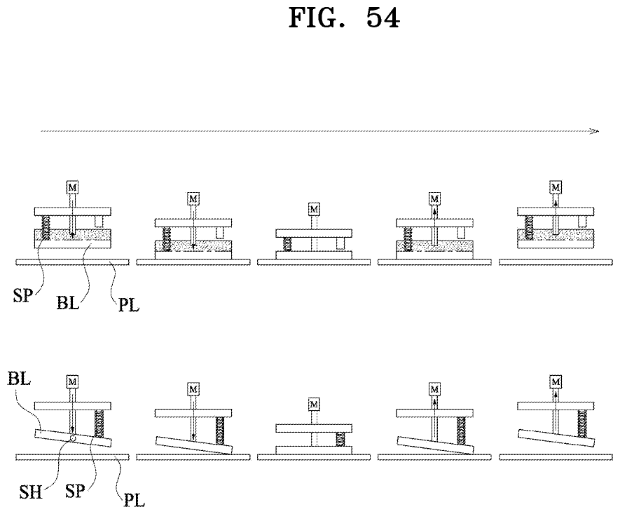

FIG. 54 illustrates an operation of a test device according to an embodiment of the present disclosure in the order of time. As illustrated in FIG. 54, a test of the sample located on the substrate PL may be performed by using a test device that includes a motor M, a patch receiving block BL, and a spring SP connected to one side of the patch receiving block BL. As illustrated in FIG. 54, since the patch receiving block BL is in oblique contact with the substrate PL and then separated therefrom, the reagent may be uniformly delivered from the patch to the sample and the damage on the sample may be minimized.

Referring to FIG. 54, in the test device, one side of the patch receiving block BL may be positioned closer to another side thereof due to the tensile force of the spring SP. Referring to FIG. 54, the patch receiving block BL may be prepared such that, due to the tensile force of the spring SP, the right side of the patch receiving block BL is oblique to the substrate PL than the left side thereof.

The preparing the patch receiving block BL by the test device may be embodied in a similar manner as used to locate the patch receiving block BL to be in the first posture by the test device.

Referring to FIG. 54, the test device may lower the patch receiving block BL toward the substrate PL to bring the patch held by the patch receiving block BL in contact with the specimen located on the substrate PL. Referring to FIG. 54, the test device may lower the patch receiving block BL toward the substrate PL to bring the patch held by the patch receiving block BL in contact with the specimen located on the substrate PL.

Referring to FIG. 54, the test device may move the patch receiving block BL to be parallel to the substrate PL. The test device may drive the motor M to make the patch receiving block BL rotate about the rotating shaft SH to be parallel to the substrate PL as the rotating shaft SH lowers. In this regard, the spring SP may be pressed as the patch receiving block BL rotates.

The moving, by the test device, the patch receiving block BL to be parallel to the substrate PL may be embodied in a similar manner as used to locate the patch receiving block BL to be in the second posture by the test device.

Referring to FIG. 54, the test device rotates the patch receiving block BL in a counterclockwise direction about the rotating shaft SH and lowers the same, at the same time, thereby sequentially bringing the patch held in the patch receiving block BL in contact with the sample located on the substrate PL from one side of the patch receiving block BL. Referring to FIG. 54, the test device may drive the motor M to sequentially bring the patch into contact with the sample from the right to the left of the patch.

Referring to FIG. 54, the test device may drive the motor M to sequentially space the patch receiving block BL from one side of the substrate PL. Referring to FIG. 54, the test device may drive the motor M to raise the rotating shaft SH upward so that the patch receiving block BL rotates clockwise with respect to the rotating shaft SH and is raised at the same time. Accordingly, the patch fixed on the patch receiving block BL may be sequentially separated from the specimen from the left side thereof.

The spacing, by the test device, the patch receiving block BL from the substrate PL sequentially may be embodied in a similar manner as used to locate the patch receiving block BL to be in the third posture by the test device.

Meanwhile, although not illustrated in FIG. 54, the test device according to the present disclosure may perform the test process according to the third example of the test method described above. The test device may perform a test process that includes the reabsorbing of the substance described in the third example of the test method.

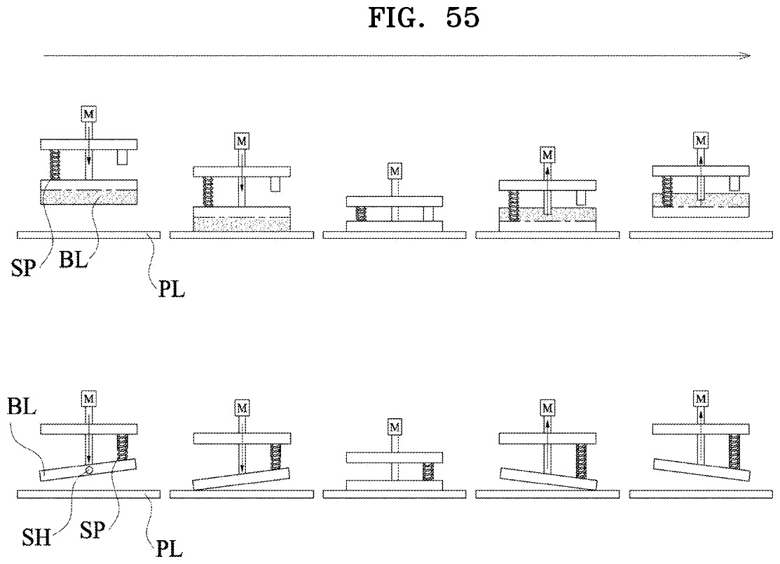

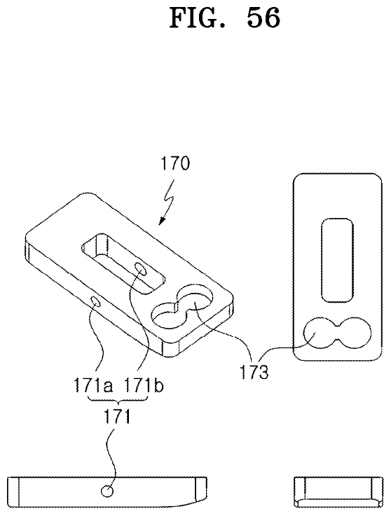

FIG. 55 illustrates an operation of a test device according to an embodiment of the present disclosure in the order of time. As illustrated in FIG. 55, a test may be performed on a sample located on the substrate PL by using a test device including a motor M, a patch receiving block BL, and a spring SP.

Referring to FIG. 55, the patch receiving block BL may be prepared such that one side thereof is closer to the substrate PL than the other side by the compressive force of the spring SP. Referring to FIG. 54, the patch receiving block BL may be prepared such that, due to the compressive force of the spring SP, the left side of the patch receiving block BL is oblique to the substrate PL than the right side thereof.

Referring to FIG. 55, the test device may lower the patch receiving block BL toward the substrate PL so that one side (for example, left side) of the patch receiving block BL is supported by the substrate PL or the sample. The test device may lower the patch receiving block BL to the substrate PL so that the left end of the patch receiving block BL which is not connected to the spring SP is supported by the substrate PL.

Referring to FIG. 55, the test device may drive the motor M to make the patch receiving block BL rotate in a clockwise direction about the rotating shaft SH to be parallel to the substrate PL as the rotating shaft SH lowers. In this regard, the spring SP may be stretched as the patch receiving block BL rotates.

Referring to FIG. 55, the test device rotates the patch receiving block BL in a clockwise direction about the rotating shaft SH and lowers the same, at the same time, thereby sequentially bringing the patch held in the patch receiving block BL in contact with the sample located on the substrate PL from the left side of the patch receiving block BL.

Referring to FIG. 55, the test device may drive the motor M to sequentially space the patch receiving block BL from one side of the substrate PL. In this regard, the patch receiving block BL may be spaced from the substrate PL sequentially from the left side of the patch receiving block BL as illustrated in FIG. 55. In this regard, the test device may drive the motor to stretch the spring SP so that the patch receiving block BL is spaced from the left side thereof. The spring SP may be stretched such that the patch receiving block BL is spaced from the left side thereof and fixed.

Meanwhile, in the embodiment described in connection with FIG. 55, the rotation direction when the patch receiving block BL moves toward the substrate is the same as the rotation direction when the patch receiving block BL is spaced away therefrom. However, embodiments of the present disclosure are not limited thereto. As illustrated in FIG. 55, even when the spring SP is a compression spring, like the embodiment illustrated in FIG. 54, the direction in which the patch is brought into contact with the sample may be different from the direction in which the patch is spaced away from the sample.

FIG. 56 illustrates a patch receiving block 170 according to an embodiment of the present disclosure. As illustrated in FIG. 65, the patch receiving block 170 may be provided as a plate having a flat bottom surface. The patch receiving block 170 may be provided as a plate having a width direction, a length direction, and a height direction.

The patch receiving block 170 may include a through hole 171 through which the rotating shaft passes.

Referring to FIG. 56, the through hole 171 may include a left through hole 171a and a right through hole 171b.

The through hole 171 may be located in the central portion of the patch receiving block 170. The through hole 171 may be located on one side of the patch receiving block 170. The through hole 171 may pass through the patch receiving block 170 in a direction parallel to the width direction of the patch receiving block 170. The through hole 171 may be located through the inside of the body part of the patch receiving block 170 or may protrude outside the body part thereof.

The patch receiving block 170 may include an elastic member contact portion 173. The elastic member contact portion 173 is located on one side of the patch receiving block 170, and may contact the elastic member. The elastic member contact portion 173 may be concave to the inside of the patch receiving block 170. The elastic member contact portion 173 is formed to have a certain depth inside the patch receiving block 170, thereby stably supporting the elastic member so as not to slip.

Referring to FIG. 56, a surface of the patch receiving block contacting the substrate may be rounded. The patch receiving block may be controlled to obliquely access the substrate from one side thereof, and in this regard, an edge of the patch receiving block accessing the substrate first may be caught by the substrate or the sample located on the substrate. Therefore, by making the edge of the patch receiving block accessing the substrate first to be rounded, this problem may be address.

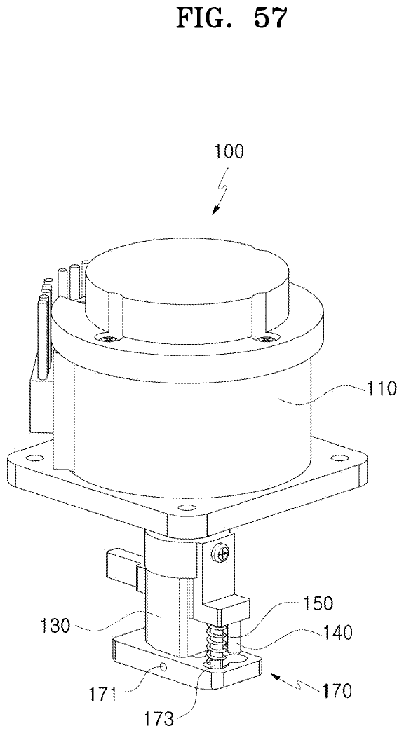

FIG. 57 illustrates a specific example of a test device 100 according to an embodiment of the present disclosure. Referring to FIG. 57, the test device 100 according to an embodiment of the present disclosure may include a motor 110, a support member 130, an elastic member 150, and a patch receiving block 170.

The motor 110 may provide power. The motor 110 may provide power in the vertical direction, that is, in the Z-axis direction. The motor 110 may provide power through the support member 130 to allow the patch receiving block 170 to move in the Z-axis direction.

Referring to FIG. 57, the support member 130 may connect the motor 110 with the patch receiving block 170. The support member 130 may be connected, on one side thereof, to a rotating shaft passing through the through hole 171 of the patch receiving block 170. The support member 130 may have one side connected to the motor 110 and extend from the motor 110 in the Z-axis direction. The support member 130 may be stretched in the Z-axis direction according to the operation of the motor 110.

Referring to FIG. 57, one side of the elastic member 150 may be connected to the support member 130. The elastic member 150 may contact one side of the patch receiving block 170 to provide a tensile force or a compressive force. The elastic member 150 may be supported by an elastic member support 140. The elastic member 150 may be detached from the elastic member support 140 as needed. The elastic member 150 may contact the elastic member contact portion 173 of the patch receiving block 170 to guide the patch receiving block 170 to be in a posture oblique to the ground.

Meanwhile, in the embodiment illustrated in FIG. 57, the elastic member 150 is a spring. However, the elastic member 150 may be any member that provides an elastic force to one side of the patch receiving block 170 to guide the patch receiving block 170 to be in an oblique posture.

The patch receiving block 170 may be the same as described on the patch receiving block 170 illustrated in FIG. 56. Referring to FIG. 57, the patch receiving block 170 may rotate around a rotating shaft penetrating the through hole 171. The patch receiving block 170 may be connected to the support member 130 through the rotating shaft and may move up and down.

Referring to FIG. 57, the elastic member is spaced from the patch receiving block and a bottom surface of the patch receiving block is parallel to the ground. However, in some embodiments, according to the control of the patch receiving block or the kind of the elastic member, the patch receiving block may be oblique to the rotating shaft in the right side or left side.

Meanwhile, although not illustrated in FIG. 57, a substrate on which the biological sample to be tested is located may be disposed under the patch receiving block 170. The substrate may be placed parallel to the ground. The controlling of the position of the patch receiving block 170 relative to the substrate may be embodied in a similar manner to that in the previous embodiments.

An embodiment of the present disclosure provides a patch control device to deliver a staining reagent used for staining to a specimen by using a gel-type patch which includes the staining reagent and a net structure forming micro-cavities for containing the staining reagent, and has a contact surface which is brought in contact with the specimen to deliver the staining reagent.

The patch control device may include a kit fixing portion to fix a kit that includes at least one patch receiving member that receives the patch in such a state that a portion of the patch is exposed, a driving portion that provides a driving force for the Z-axis direction movement of the patch receiving member, a support member extending in the Z-axis direction from the driving portion, and a spring member fixed such that one end of the support member moves integrally with the support member.

The description of the patch receiving block described above may be applied to the patch receiving member. The bottom surface of the patch receiving block may be provided with an edge thereof on the one side which is rounded to guide the bottom surface of the patch is sequentially brought into contact with the specimen form the one side thereof.

The driving portion may be the same as described in connection with the motor above. The driving portion may lower the support member in the Z-axis direction such that the patch contacts the specimen. The driving portion may raise the support member in the Z-axis direction such that the patch is separated from the specimen.

The support member may be connected, at an end thereof, to a point of the patch receiving member such that, due to the driving force of the driving portion, the position of the point of the patch receiving member with respect to the X axis may be changed. The support member may be connected, at the end thereof, to the patch receiving member such that the patch receiving member freely rotates about the rotating shaft in the X-axis direction perpendicular to the Z-axis direction.

The rotating shaft may be connected to one side of the support member and provided to pass through the center of the patch receiving member. The rotating shaft may be connected to one side of the support member and may be connected to a surface of the patch receiving member such that the patch receiving member rotates around an external axis.

The spring member has another end which is fixed at a point spaced apart from the rotating shaft of the patch receiving member at a certain distance. The spring member may apply a spring force between the one end and the other end of the patch receiving member so that the patch receiving member is positioned oblique to the substrate while the patch held in the patch receiving member is not in contact with the specimen.

The spring member may be deformed such that a surface of the patch receiving member is parallel to the substrate as the patch is in contact with the specimen as the patch receiving member rotates along the substrate on which the specimen is located.

Due to the rotation of the patch receiving member about the X axis-direction rotating shaft during the patch is not in contact with the specimen, the spring member may provide a tensile force in the Z-axis direction so that the patch receiving member is maintained in the oblique posture with respect to the Y axis perpendicular to the Z axis and the X axis. The spring member may be pressed such that a surface of the patch receiving member is parallel to the substrate as the patch receiving member rotates along the substrate on which the specimen is located by the driving of the driving portion. In this regard, a side of the patch receiving member which is connected to the spring member may be provided to have a round edge.

Due to the rotation of the patch receiving member about the X axis-direction rotating shaft during the patch is not in contact with the specimen, the spring member may provide a compressive force in the Z-axis direction so that the patch receiving member is maintained in the oblique posture with respect to the Y axis perpendicular to the Z axis and the X axis. The spring member may be stretched such that a surface of the patch receiving member is parallel to the substrate as the patch receiving member rotates along the substrate on which the specimen is located by the driving of the driving portion. In this regard, a side of the patch receiving member which is opposite to the side thereof connected to the spring member may be provided to have a round edge.

The spring member may be separated from the patch receiving member according to the degree of rotation of the patch receiving member. In this regard, the spring member is brought in contact with a spring contact portion, which is concaved inward the patch receiving member and is located a certain distance away from the rotating shaft of the patch receiving member in the direction perpendicular to the X axis according to the degree of rotation of the patch receiving member, so as to provide a tensile force to the patch receiving member in the Z-axis direction.

### 8.3 Second Embodiment: Two Rotating Shafts

A test device according to one embodiment of the present disclosure may include a patch receiving block and a motor. The test device may test a sample using a patch receiving block provided to be rotatable with respect to a first rotating shaft and a second rotating shaft.

The patch receiving block may include a patch receiving block arranged to be rotatable about each of the first rotating shaft and the second rotating shaft which are parallel to the Y-axis direction. The first rotating shaft and the second rotating shaft may be located at an end of one side of the patch receiving block and an end of the other side thereof, respectively.

The position of each of the first rotating shaft and the second rotating shaft may be controlled by the motor. The first rotating shaft and the second rotating shaft are connected to a support member, and may receive power from the motor through the support member.

The motor may control the position of each of the first rotating shaft and the second rotating shaft in the Z-axis direction. The motor may control positions of the first and second rotation shafts to prepare the patch receiving block to be oblique with respect to the substrate.

The motor may include the first motor and the second motor. The first motor may be connected to the first rotating shaft through a first support member, and may control the position of the first rotating shaft through the support member. The second motor may be connected to the second rotating shaft through a second support member and control the position of the second rotating shaft through the second support member.

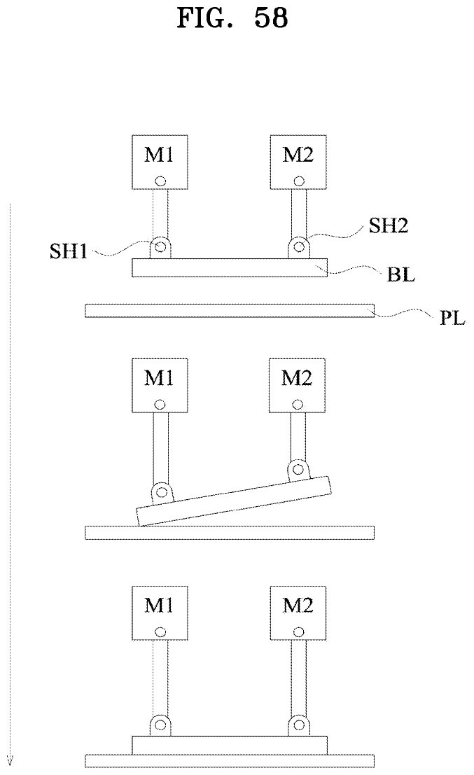

FIG. 58 illustrates an operation of a test device according to an embodiment of the present disclosure.

Referring to FIG. 58, a sample may be tested by using a test device including a first motor M1, a second motor M2, a first rotating shaft SH1, a second rotating shaft SH2, and a patch receiving block BL.

Referring to FIG. 58, the test device may be prepared such that a bottom surface of the patch receiving block BL is to be parallel to the substrate PL or the ground.

Referring to FIG. 58, the test device may drive the first motor M1 to lower the first rotating shaft SH1. The test device may cause the patch receiving block BL to rotate in a counterclockwise direction with respect to the second rotating shaft SH2 by a certain distance by lowering the first rotating shaft SH1. Referring to FIG. 58, the test device, by driving the first motor M1, may rotate the patch receiving block BL to allow an end of the left side of the patch receiving block BL to be supported by the substrate PL.

Referring to FIG. 58, the test device may drive the second motor M2 to lower the second rotating shaft SH2. The test device may lower the second rotating shaft SH1 so that the patch receiving block BL rotates in a clockwise direction with respect to the first rotating shaft SH1 by a certain distance. Referring to FIG. 58, the patch receiving block BL may rotate with respect to the first rotating shaft SH1 and lower at the same time as the second motor M2 is driven. The posture of the patch receiving block BL may be changed such that the bottom surface thereof is parallel to the substrate PL when the second motor M2 is driven. The test device may drive the second motor M2 such that the patch receiving block BL is brought into contact with the specimen located on the substrate PL from one side of the patch receiving block BL, for example, the left side thereof.

Although not illustrated in FIG. 58, the test device may space the patch receiving block BL from the substrate PL. The test device may drive the first motor M1 or the second motor M2 to space the patch receiving block BL from the substrate PL from one side of the patch receiving block BL. In an embodiment, the test device may drive the second motor M2 to space the patch receiving block BL from the substrate PL from the right side of the patch receiving block BL. In this regard, the test device may control the driving of each of the first motor M1 and the second motor M2 such that with respect to the whole area of the sample, the time duration in which the sample is in contact with the patch is constant.

In the embodiment illustrated in FIG. 58, the bottom surface of the patch receiving block BL is arranged to be parallel to the substrate PL or the ground, and then, due to the driving of the motor, the bottom surface of the patch receiving block BL is changed to be oblique with respect to the substrate PL or the ground by driving the motor. However, embodiments of the present disclosure are not limited thereto. The test device may prepare the patch receiving block BL to have such a bottom surface that is oblique to the substrate PL or the ground.

Although not illustrated in FIG. 58, the test device according to the present disclosure may perform a test process according to the third example of the test method described above. The test device may perform a test process that includes the reabsorbing of the substance described in the third example of the test method.

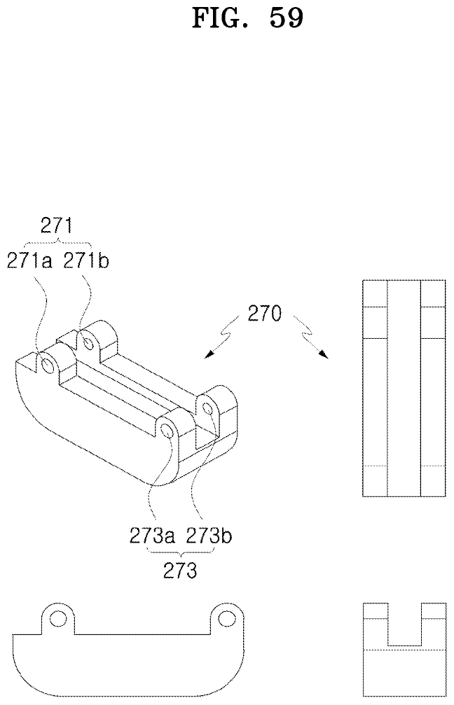

FIG. 59 illustrates a patch receiving block 270 according to an embodiment of the present disclosure. Like the patch receiving block described in the embodiments, the patch receiving block 270 may receive a patch containing a reagent in such a way that one surface of the patch is exposed or protrudes.

Referring to FIG. 59, the patch receiving block 270 may include a first through-hole 271 through which a first rotating shaft passes and a second through-hole 273 through which a second rotating shaft passes. The first through-hole 271 may include a left first through-hole 271a and a right first through-hole 271b. The second through-hole 273 may include a left second through-hole 273a and a right second through-hole 273b.

Referring to FIG. 59, the bottom surface of the patch receiving block may have round edges. The round edges may be used to prevent the edges from being caught by, for example, the substrate or scratching the specimen when the patch receiving block is obliquely brought into contact with the substrate and is separated therefrom.

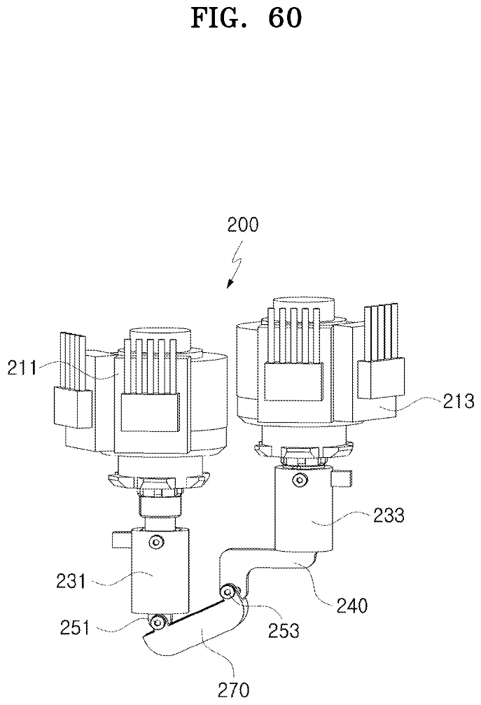

FIG. 60 illustrates a specific example of a test device 200 according to an embodiment of the present disclosure. Referring to FIG. 60, the test device 200 according to an embodiment of the present disclosure includes a first motor 211, a second motor 213, a first support member 231, a second support member 233, a first rotating shaft 251, a second rotating shaft 253, and a patch receiving block 270.

The first motor 211 may provide a driving force to the first support member 231. The first motor 211 may provide power to move the first support member 231 in the Z-axis direction. The first motor 211 may provide power to stretch the first support member 231 in the Z-axis direction. The first motor 211 may generate a displacement in the Z-axis direction to the first rotating shaft 251 through the first support member 231.

The second motor 213 may provide a driving force to the second support member 233. The second motor 213 may provide power to move the second support member 233 in the Z-axis direction. The second motor 213 may provide power to stretch the second support member 233 in the Z-axis direction. The second motor 213 may generate a displacement in the Z-axis direction to the second rotating shaft 253 through the second support member 233.

Referring to FIG. 60, the test device 200 may further include a connection member 240 for connecting the second support member 233 with the patch receiving block 270. In this regard, the second motor 213 may provide power to change the position of the second rotating shaft 253 connected to the patch receiving block 270 through the connection member 240 connected to the second support member 233.

However, the functions of the first motor 211 and the second motor 213 are not limited to the embodiments described above, and the first motor 211 and the second motor 213 may provide power necessary for controlling the patch receiving block 270 in various manners.

Referring to FIG. 60, the patch receiving block 270 may be connected to the first support member 231 and the connection member 240 to receive power. The patch receiving block 270 may rotate about the second rotating shaft as the first support member 231 moves up and down. The patch receiving block 270 may rotate about the second rotating shaft as the second support member 233 moves up and down.

Meanwhile, although not illustrated in FIG. 60, a substrate on which the biological sample to be tested is located may be disposed under the patch receiving block 270. The substrate may be placed parallel to the ground. The controlling of the position of the patch receiving block 270 relative to the substrate may be embodied in a similar manner to that in the previous embodiments.

According to an embodiment of the present disclosure, the test device may include a patch receiving member that receives the patch of which at least a portion is exposed, a driving portion which is connected to a support member and provides a driving force for the movement of the support member in the Z-axis direction, a first support member extending from the driving portion in the Z-axis direction, and a second support member extending from the driving portion in the Z-axis direction.

The patch receiving member may move toward the substrate on which the specimen is located according to the motion of the driving portion to bring the contact surface of the patch into contact with the specimen.

When the first support member lowers in the Z-axis direction by the driving portion, the patch receiving member may rotate about the second rotating shaft in a first direction so that a side of the patch receiving member is oblique toward the substrate on which the specimen is located. When the second support member lowers in the Z-axis direction, the patch receiving member may rotate about the first rotating shaft in a second direction so that another side of the patch receiving member is oblique toward the substrate on which the specimen is located and thus the contact surface of the patch is brought into contact with the specimen from the side to the other side of the patch receiving member.

The first support member may extend from the driving portion in the Z-axis direction, and may be connected to one side of the patch receiving member at an end thereof so that, due to the driving force of the driving portion, the first support member changes the position of the side of the patch receiving member in the Z axis. The first support member may be connected, at the end thereof, to the patch receiving member such that the patch receiving member freely rotates about the first rotating shaft in the X-axis direction perpendicular to the Z-axis direction.

The second support member may extend from the driving portion in the Z-axis direction, and may be connected to another side facing the side of the patch receiving member at an end thereof so that, due to the driving force of the driving portion, the second support member changes the position of the other side of the patch receiving member in the Z axis. The second support member may be connected, at the end thereof, to the patch receiving member such that the patch receiving member freely rotates about the second rotating shaft in the X-axis direction perpendicular to the Z-axis direction.

The driving portion may lower the first support member in the Z-axis direction such that, during the other side of the patch receiving member is spaced from the substrate by a certain distance, the side of the patch receiving member is oblique to the substrate so that a side of the contact surface is brought into contact with the specimen first and then another side thereof.

The driving portion may lower the second support member in the Z-axis direction such that, during the side of the patch receiving member is supported by the substrate, the patch receiving member rotates about the first rotating shaft by a certain distance to allow the other side of the patch receiving member to access the substrate.

Meanwhile, the driving portion may include a first driving portion and a second driving portion.

In this regard, the patch receiving member obliquely accesses the substrate from one side of the patch receiving member according to the operation of the first driving portion, and the obliquely accessing the substrate by the patch receiving member may include rotating the patch receiving member about the second rotating shaft such that one side of the patch receiving member is oblique to the substrate on which the specimen is located as the first support member lowers in the Z-axis direction by the first driving portion.

The patch receiving member, while obliquely accessing the substrate according to the operation of the first driving portion, rotates such that one side thereof is parallel to the substrate according to the operation of the second driving portion and brings the patch to be in contact with the specimen. In this regard, the bringing, by the patch receiving member, the patch to be in contact with the specimen may include rotating, about the first rotating shaft, the patch receiving member such that the other side of the patch receiving member accesses the substrate on which the specimen is located so that the contact surface of the patch is brought into contact with the specimen from one side to the other side, as the second support member lowers in the Z-axis direction by the second driving portion.

The rotating the patch receiving member such that, during the patch is in contact with the specimen, the patch receiving member rotates to be oblique to the substrate according to the operation of the first driving portion, may include rotating the patch receiving member around the second rotating shaft away from the substrate on a side thereof, so that the contact surface of the patch is separated from the specimen from the side to another side of the patch.

When the first support member is raised in the Z-axis direction by the first driving portion during the other side of the contact surface of the patch is in contact with the specimen, the patch receiving member may rotate around the second rotating shaft in the second direction such that the contact surface is separated from the specimen from the side to the other side of the contact surface of the patch.

The rotating around the second rotating shaft in the second direction may be controlling the rotation of the patch receiving member such that the contact time during which the specimen is in contact with the patch is constant in the whole area of the specimen.

When one side of the patch receiving member accesses the substrate and makes the contact with the specimen from the side of the contact surface, the edge of the patch receiving member on the side thereof may be rounded to allow the contact surface of the patch is brought into the contact with the specimen from the side to the other side of contact surface of the patch.

When the patch receiving member is spaced from the specimen from the side thereof, the edge of the patch receiving member on the other side thereof may be rounded so that the contact surface of the patch is separated from the specimen from the side to the other side thereof without being caught by the specimen.

### 8.4 Third Embodiment: Device Using Kit

The test devices described above may include a test kit that includes a patch receiving member that receives the patch and a pressing head that presses the patch receiving member, and may perform the test method.

According to an embodiment of the present disclosure, the patch receiving block does not receive the patch, and may compress a patch receiving member in which the patch is placed to make the patch receiving block to be in indirect contact with the specimen or the substrate. Hereinafter, as described above, a member that delivers a pressing force to the patch receiving member and is in an indirect contact with the specimen or substrate through the patch receiving member, may be defined as a pressing head.

Hereinafter, the configuration and operation of the kit, the pressing head, and a test device including the kit and the pressing head will be described.

### 8.4.1 Test Kit

According to an embodiment of the present disclosure, the test device described above may be used to test a specimen by using a test kit (hereinafter, referred to as a kit) that includes at least one patch receiving member.

The kit may include a frame supporting a patch receiving member which is inserted into the test device and receives the patch.

The frame may include at least one patch receiving member housing portion in which the patch receiving member is positioned. The patch receiving member housing portion may include a patch receiving member support that supports the patch receiving member. The patch receiving member housing portion may have a recess that engages with the protrusion of the patch receiving member and prevents disengagement of the patch receiving member.

The kit may include at least one patch receiving member to receive a patch that contains a reagent required for testing the specimen. The kit may include a plurality of patch receiving members. The patch receiving members may respectively include patches containing a plurality of types of reagents required for testing the specimen.

The kit may include a patch receiving member that is fixed to the frame and receives a patch that contains a test reagent used for testing of the specimen.

The patch receiving member may include a patch fixing portion to fix the patch. The patch receiving member may have an open bottom surface. The patch receiving member may fix the patch such that a bottom surface of the patch is exposed by the open bottom surface of the patch receiving member.

The patch receiving member may further include an elastic portion. The patch receiving member may include the elastic portion on both sides thereof. Regarding the elastic portion, when a force is applied to the patch receiving member downward the kit, the elastic force may act on the patch receiving member upward the kit. The elastic portion may support the patch receiving member against the kit so that the patch receiving member is fixed in a certain position of the kit.

The elastic portion may be formed to extend from both sides of the top plate of the patch receiving member. The elastic portion may be formed to extend from one side and another side of each of edges on opposite sides of a top plate of the patch receiving member toward the center of a bottom portion of a side surface of each of opposite sides of the patch receiving member. The elastic portion may be provided as a leaf spring.

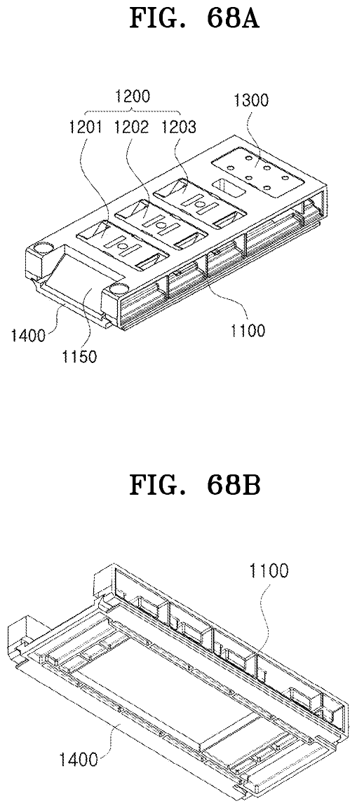

FIG. 68 illustrates an example of a frame 1100 according to an embodiment of the present disclosure.

Referring to FIG. 68, the frame 1100 may include a first patch receiving member housing portion 1111a, a second patch receiving member housing portion 1111b, and a third patch receiving member housing portion 1111c. A first patch receiving member, a second patch receiving member, and a third patch receiving member may be fixed to the first patch receiving member housing portion 1111a, the second patch receiving member housing portion 1111b, and the third patch receiving member housing portion 1111c, respectively. The first to third patch receiving members may receive patches that contain different reagents.

The first patch receiving member housing portion 1111a, the second patch receiving member housing portion 1111b, and the third patch receiving member housing portion 1111c may each have a recess 1115 that engages with the protrusions provided in the first to third patch receiving members. The patch receiving member housing portion 1110 may fix the patch receiving members not to disengage from the frame 1110 by using the recess.

The patch receiving member housing portion 1110 may include a patch receiving member support 1113. The patch receiving member support 1113 may support a patch receiving member. The patch receiving member support 1113 may be positioned on opposite sides of the patch receiving member housing portion 1110 in the Y-axis direction to support the opposite sides of the patch receiving member.

The patch receiving member support 1113 may support elastic portions provided on opposite sides of the patch receiving member. The patch receiving member support 1113 may limit the range of movement of the patch receiving member within the frame 1100.

Referring to FIG. 68, the frame 1100 may further include a medium receiving member housing portion 1130. The medium receiving member housing portion 1130 may receive a medium receiving member.

The medium receiving member housing portion 1130 may include a medium receiving member support. The medium receiving member support may be provided as a step with respect to the bottom surface of the frame.

The medium receiving member support may support the medium receiving member. The medium receiving member support may support a storage medium coupled to the medium receiving member.

The medium receiving member support may be positioned on opposite sides of the medium receiving member housing portion 1130 in the Y direction to support opposite sides of the medium or the medium receiving member. The medium receiving member support may be provided symmetrically on opposite sides of the medium receiving member housing portion 1130.

The medium receiving member housing portion 1130 may include at least one hole 1131. The medium receiving member support may be positioned on opposite sides of the at least one hole 1131.

The medium receiving member housing portion 1130, the medium receiving member, the medium, and the hole 1131 will be described in more detail with reference to a fifth embodiment to be described later.

Referring to FIG. 68, the frame 1100 may further include a film attachment portion 1150. The film attachment portion 1150 may be provided as an inclined surface extending from the top surface of the frame. A smear film for smearing a sample to be tested may be attached on the film attachment portion 1150.

Referring to FIG. 68, the frame 1100 may include a sliding rail 1170. The frame 1100 may include the sliding rail 1170 for sliding along a separately provided guide rail.

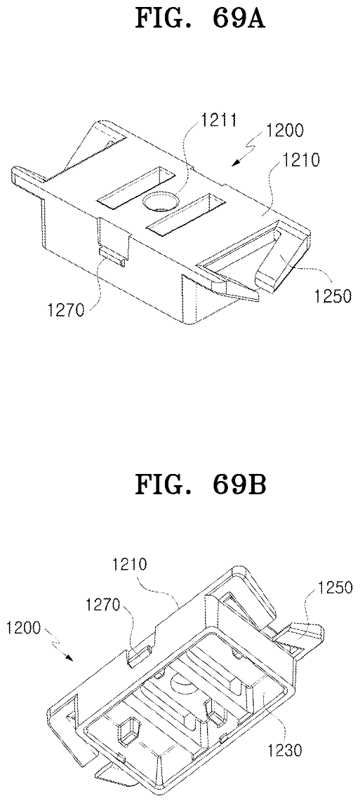

(a) and (b) of FIG. 69 illustrate an example of a patch receiving member 1200 according to an embodiment of the present disclosure. The patch receiving member 1200 may be provided as being coupled to the kit described above. Referring to FIG. 69, the patch receiving member 1200 may have a top surface 1210 which is flat.

A discharge port 1211 may be provided on the top surface 1210 of the patch receiving member 1200.

Referring to FIG. 69, the patch receiving member 1200 may have a bottom surface which is open. The patch receiving member 1200 may receive the patch such that at least a portion of the patch is exposed to the bottom surface thereof.

Referring to FIG. 69, the patch receiving member 1200 may have a cavity 1230 surrounded by the top surface 1210 and side walls. The patch may be placed in the cavity 1230. The cavity 1230 may have at least one protruding structure protruding from the inner surfaces of the sidewalls to prevent the disengagement of the patch. The patch may be discharged as a liquid through the discharge port 1211 of the patch receiving member 1200, and solidified in the cavity 1230.

Referring to FIG. 69, the patch receiving member 1200 may have an elastic portion 1250 provided as a flat spring. The elastic portion 1250 may be provided on the frame such that when the shape of the elastic portion 1250 is deformed by a pressing force, the contact surface of the patch protrudes further than the bottom surface of the frame of the kit.

The elastic portion 1250 may include a support portion extending from the edge of each of opposite sides of the top surface 1210 and an elastic portion extending from the support portion to the center of a lower portion of the side surface of the patch receiving member 1200. The elastic part may exert an elastic force upward when the patch receiving member 1200 is pressed downward.

Referring to FIG. 69, the patch receiving member 1200 may include a protrusion 1270. The protrusion 1270 may be combined with a recess formed in the frame of the kit. The protrusion 1270 may be formed on the outer surface of the sidewall forming the cavity.

In the embodiment illustrated in FIG. 69, the protrusion 1270 is formed on the patch receiving member 1200. However, the embodiment is an example only, and the protrusion 1270 may be replaced with other structure for fixing the patch receiving member 1200 on the frame.

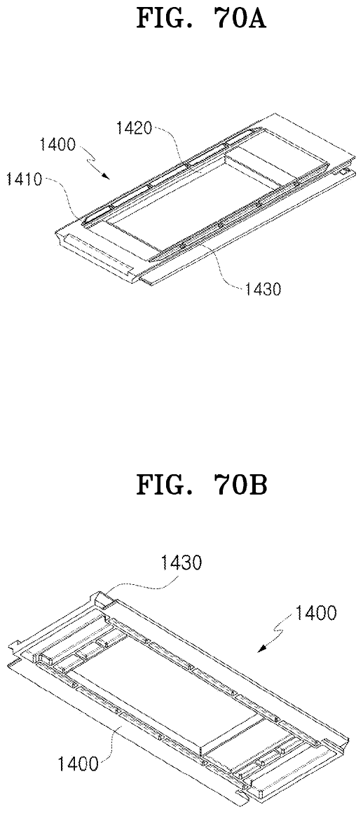

FIG. 70 illustrates a view of a base 1400 according to an embodiment of the present disclosure. The base 1400 may be included in the kit described above. The base 1400 may be combined with the frame. Referring to (a) and (b) of FIG. 70, the base 1400 may include a substrate receiving portion 1410, a window 1420, and a guide rail 1430.

The substrate receiving portion 1410 may receive a substrate on which the specimen is positioned. The substrate receiving portion 1410 may fix a substrate on which the specimen is located.

The window 1420 may be formed under the substrate receiving portion 1410. The window 1420 may be formed to expose a portion of the substrate placed in the substrate receiving portion 1410 on which the specimen is located. The window 1420 may be formed in the center of the substrate receiving portion 1410 so that the portion of the substrate on which the specimen is located is exposed.

The guide rail 1430 may be located on opposite sides of the substrate receiving portion 1410. The guide rail 1430 may be formed parallel to the length direction of the base 1400. The guide rail 1430 may be formed on opposite sides of the base 1400. The guide rail 1430 may be coupled to the sliding rail 1170 of the frame 1100. The guide rail 1430 may be coupled to the sliding rail 1170 to provide a movement path for the base 1400 of the frame 1100.

The base 1400 may be located above an optical module of the test device disclosed according to the present specification. The base 1400 may be located adjacent to the optical module to facilitate optical observation of the specimen positioned on the substrate through the window 1420.

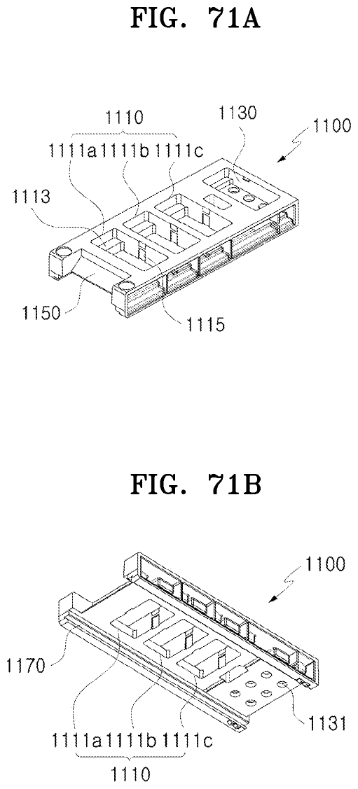

FIG. 71 illustrates a view of a kit according to an embodiment of the present disclosure. Referring to FIG. 71, a kit according to an embodiment of the present disclosure may include the frame 1100, the patch receiving member 1200 which is coupled to the frame 1100 and receives a patch for containing a reagent used when a specimen is tested, the medium receiving member which is coupled to the frame 1100 and receives a storage medium for containing a fixing reagent, and the base 1400 which is coupled to the frame 1100 and receives a substrate.

The patch receiving member 1200 may include a first patch receiving member 1201 to receive a first patch, a second patch receiving member 1202 to receive a second patch, and a third patch receiving member 1203 to receive a third patch.

The first patch receiving member 1201, the second patch receiving member 1202, and the third patch receiving member 1203 may include patches containing different reagents. The first patch receiving member 1201, the second patch receiving member 1202, and the third patch receiving member 1203 may be arranged in order in the frame 1100 to correspond to the order in which the respective reagents are used.

The frame 1100 may slide against the base 1400. The frame 1100 may be coupled such that the sliding rail described above engages with the guide rail of the base 1400. The frame 1100 may slide against the base 1400 as the sliding rail moves back and forth along the guide rail.

The frame may further include the smear film attachment portion 1150 described above. The smear film attachment portion 1150 may have an inclined surface. On the smear film attachment portion 1150, a smear film in which a specimen (for example, blood) is smeared on the substrate, may be attached with a downward slope along the inclined surface thereof.

### 8.4.2 3-1 Embodiment: Kit+Pressing Head+Spring

In this regard, some embodiments of a device for testing a specimen using the kit described above will be described.

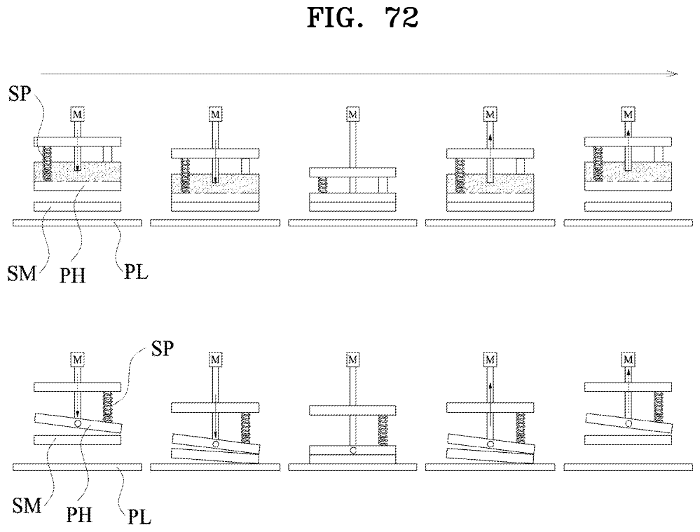

FIG. 72 illustrates an operation of a test device according to an embodiment of the present disclosure in the order of time. Referring to FIG. 72, the test device may include a motor M, a patch receiving member SM, a pressing head PH, and a spring SP connected to one side of the pressing head PH.

The test device may test the specimen located on the substrate PL by using the motor M providing power, the patch receiving member SM to receive the patch, the pressing head PH, and the spring SP. In detail, the test device may obliquely accesses the patch receiving member to the substrate PL by using the motor M and the pressing head PH, and then separate the patch receiving member from the substrate PL, thereby uniformly delivering, to the specimen, the reagent contained in the patch and increasing the efficiency of the test.

The patch receiving member SM described in the present embodiment may be included in the kit described above. The patch receiving member SM is fixed to the frame of the kit, and the relative position thereof with respect to the frame may change as the pressing head PH lowers.

Referring to FIG. 72, the test device may be prepared such that one side of the pressing head PH is closer to the substrate PL or the patch receiving member SM than the other side thereof. The pressing head PH may be prepared as having a posture oblique to the substrate PL or the patch receiving member SM by the spring SP. Referring to FIG. 71, the pressing head PH may be prepared as being oblique to the substrate PL such that the right side of the pressing head PH is closer to the substrate PL than the left side thereof.

For example, the pressing head PH and/or the patch receiving member SM may be prepared as having the first posture described in the previous embodiments of the test method described above.

Referring to FIG. 72, the test device may lower the pressing head PH toward the substrate PL. The test device may control the pressing head PH such that as the pressing head PH lowers obliquely, the patch receiving member SM lowers obliquely toward the substrate by the pressing head PH. Referring to FIG. 71, the test device may control the pressing head PH such that one side of the pressing head PH applies a pressing force on one side of the patch receiving member SM to make the patch receiving member SM to be inclined.

When the pressing force is applied to the side (for example, the right side) of the patch receiving member SM by the pressing head PH, an elastic portion on the side of the patch receiving member SM may be deformed.

Referring to FIG. 72, the test device may control the pressing head PH and the patch receiving member SM to be parallel to the substrate PL. Referring to FIG. 72, the test device may lower the pressing head PH such that the pressing head PH is supported by the patch receiving member SM and the substrate PL to rotate in a counterclockwise direction with respect to the rotating shaft of the pressing head PH. Referring to FIG. 72, the test device may control the pressing head PH such that the patch receiving member SM accesses the substrate from the right side thereof to bring the patch in contact with the specimen from the right side to the left side of the patch. As the pressing head PH lowers, the pressing head PH and the patch receiving member SM may rotate along the substrate PL. The spring SP may be pressed as the pressing head PH rotates. The pressing head PH and/or the patch receiving member SM may be provided in the second posture described in the embodiments of the test method described above.

Since the pressing head PH rotates in the state in which one side (for example, the right side) of the patch receiving member SM is pressed toward the substrate and thus the elastic portion of the side thereof is deformed, the elastic portion on the other side (for example, the left side) of the patch receiving member SM may be deformed.

Referring to FIG. 72, the test device may raise the pressing head PH such that the patch receiving member SM is sequentially spaced from the substrate PL from the right side of the patch receiving member SM. The test device may control the pressing head PH such that, in the state in which one side (for example, the right side) of the patch receiving member SM is supported by the substrate PL, the patch receiving member SM rotates in one direction (for example, a clockwise direction) to sequentially separate the patch form the specimen in one direction (for example, from the left side to the right side). The pressing head PH and/or the patch receiving member SM may be changed to have the third posture described in the embodiments of the test method described above.

In the state in which the elastic portion on the side of the patch receiving member SM is deformed, the pressing head PH rotates. Accordingly, the elastic portion on the other side (for example, the left side) of the patch receiving member SM may return to the original state.

Meanwhile, the operation of the test device may be understood in a similar manner as described in connection with the embodiments described with reference to FIG. 54. However, in the embodiments described in FIG. 54, the test device directly controls the patch receiving block that receives the patch, whereas in the embodiment described in FIG. 72, the test device controls the pressing head, which does not receive the patch, such that the patch receiving member receiving the patch is lowered through the pressing head. Accordingly, the controlling the pressing head PH by the test device may be embodied in a similar manner to the embodiment described in connection with FIG. 54 in which the test device controls the patch receiving block.

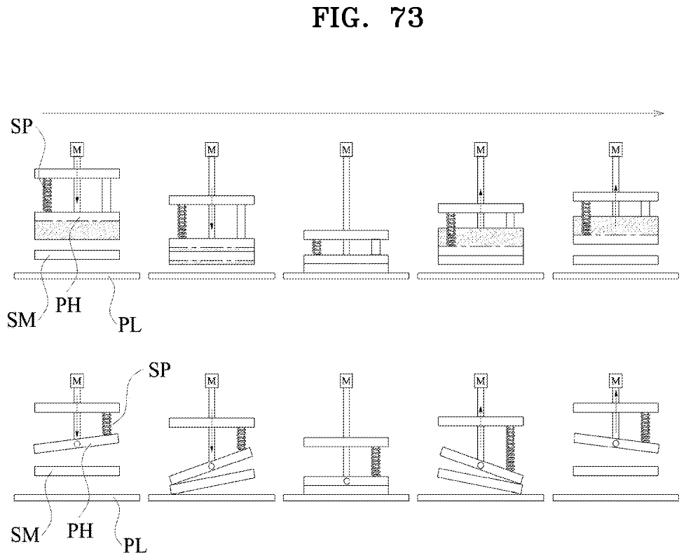

FIG. 73 illustrates an operation of a test device according to an embodiment of the present disclosure in the order of time. Referring to FIG. 73, the test device may include a motor M, a patch receiving member SM, a pressing head PH, and a spring SP connected to one side of the pressing head PH. Hereinafter, the embodiment illustrated in FIG. 73 will be described with reference to FIGS. 55 and 72.

Referring to FIG. 73, the pressing head PH may operate in a similar manner as the patch receiving block described in the embodiment of the device described in connection with FIG. 55 according to the first embodiment. In other words, the test device may control the pressing head PH to obliquely access the patch receiving member SM. In this regard, the controlling the pressing head PH by the test device may be performed in a similar manner as used for the device illustrated in FIG. 55 to control the patch receiving block to be brought into oblique contact with the specimen.

Referring to FIG. 73, like the test device illustrated in FIG. 72, the test device may obliquely lower the pressing head PH toward the substrate PL. The test device may be prepared such that one side of the pressing head PH (for example, the left side) is closer to the substrate PL or the patch receiving member SM than the other side (for example, the right side) thereof, and may control such that the pressing head PH lowers toward the substrate PL, the patch receiving member SM is supported by the substrate from one side (for example, left side) thereof, and the pressing head PH and the patch receiving member SM rotate to be parallel with the substrate PL.

Referring to FIG. 73, the test device may control the pressing head PH and the patch receiving member SM to rotate to be parallel to the substrate PL, thereby stretching the spring SP.

When the test device lowers the pressing head PH, a pressing force may be applied to one side (for example, the left side) of the patch receiving member SM by the pressing head PH, so that the elastic portion of the side of the patch receiving member SM may be deformed. Since the pressing head PH rotates in the state in which one side (for example, the left side) of the patch receiving member SM is pressed toward the substrate and thus the elastic portion of the side thereof is deformed, the elastic portion on the other side (for example, the right side) of the patch receiving member SM may be deformed.

Referring to FIG. 73, the test device may control the pressing head PH such that, to uniformly deliver the sample to the specimen, the patch receiving member SM is spaced from the substrate PL from the side (for example, the left side) of the patch receiving member SM which accesses the substrate PL first).

The spring SP may be maintained as being stretched so that the patch receiving block BL is spaced from the left side thereof.

As illustrated in FIG. 73, even when the spring SP is a compression spring, like the embodiment illustrated in FIG. 72, the direction in which the patch is brought into contact with the sample may be different from the direction in which the patch is separated away from the sample.

An embodiment of the present disclosure provides a patch control device to deliver a staining reagent used for staining to a specimen by using a gel-type patch which includes the staining reagent and a net structure forming micro-cavities for containing the staining reagent, and has a contact surface which is brought in contact with the specimen to deliver the staining reagent.

The patch control device may include a substrate fixing unit, a kit receiving unit, a pressing head, a driving portion, a support member, and a spring member. The general configuration and operation of the substrate fixing unit, the driving portion, the support member, and the spring member may be implemented in a similar manner to that described in connection with the device in the first embodiment described above.

The kit receiving unit may receive a patch receiving kit that includes at least one patch receiving member that receives the patch of which at least a portion is exposed. The kit receiving unit may fix the patch receiving kit to a position which is spaced from the substrate by a certain distance.

The patch receiving member may further include an elastic portion, and the elastic portion may exert an elastic force on the patch receiving member in a direction away from the specimen. The elastic portion may be provided as a leaf spring positioned on opposite sides of the patch receiving member and may apply, to the patch receiving member, an elastic force acting in one direction against the patch receiving kit.

The pressing head may provide a pressing force to the patch receiving member by being in contact with the patch receiving member. The pressing head may be brought into contact with one side of the patch receiving member to provide a pressing force, so that as the driving portion is driven, the patch receiving member obliquely accesses the surface of the substrate on which the specimen is located to make the patch be in contact with the specimen from one side of the patch.

The bottom surface of the pressing head may be provided with an edge rounded to prevent the edge of the pressing head from getting caught by the patch receiving member.

The driving portion may provide the driving force for movement of the pressing head in the Z-axis direction. The driving portion may lower the support member in the Z-axis direction so that the pressing head and the patch receiving member in contact with the pressing head lower and the patch is brought in contact with the specimen.

The driving portion may raise the support member in the Z-axis direction such that the patch is separated from the specimen as the pressing head lowered by the support member is raised and thus the patch receiving member pressed by the pressing head is raised, thereby separating the patch from the specimen.

The support member may be connected to the pressing head such that the support member extends from the driving portion in the Z-axis direction, may be connected, at an end thereof, to one point of the pressing head so that due to the driving force of the driving portion, the position of the point of the pressing head with respect to the Z axis is changed, and at the end, the pressing head freely rotates around the rotating shaft in the X-axis direction perpendicular to the Z-axis direction.

The spring member is fixed such that one end thereof moves integrally with the support member and another end thereof is fixed at a position spaced a certain distance from the rotating shaft of the pressing head, and, while the patch placed in the pressing heads is not in contact with the specimen, the spring member may apply a spring force between the end and the other end to make the pressing head be oblique to the substrate.

The spring member may be deformed to make one surface of the pressing head be parallel to the surface of the substrate, as the side of the pressing head is supported by the substrate on which the specimen is located through the patch receiving member, and due to the pressing force provided by the pressing head, the patch receiving member rotates to be parallel to the surface of the substrate.

Due to the rotation of the pressing head about the X axis-direction rotating shaft during the pressing head is not in contact with the patch receiving member, the spring member may provide a tensile force in the Z-axis direction so that the spring member is maintained in the oblique posture with respect to the Y axis perpendicular to the Z axis and the X axis.

The spring member may be compressed such that one surface of the pressing head is parallel to the surface of the substrate, due to the rotation of the pressing head together with the patch receiving member following the top surface of the surface of the substrate on which the specimen is located by the driving of the driving portion.

Due to the rotation of the pressing head about the X axis-direction rotating shaft during the pressing head is not in contact with the patch receiving member, the spring member may provide a compressive force in the Z-axis direction so that the spring member is maintained in the oblique posture with respect to the Y axis perpendicular to the Z axis and the X axis.

The spring member may be stretched such that one surface of the pressing head is parallel to the surface of the substrate, due to the rotation of the pressing head together with the patch receiving member following the top surface of the surface of the substrate on which the specimen is located by the driving of the driving portion.

The spring member may be spaced from the pressing head depending on the degree of rotation of the pressing head. The spring member is brought in contact with a spring contact portion, which is concaved inward the pressing head and is located a certain distance away from the rotating shaft of the pressing head in the direction perpendicular to the X axis according to the degree of rotation of the pressing head, so as to provide a tensile force to the pressing head in the Z-axis direction.

The rotating shaft may be connected to one side of the support member and provided to pass through the center of the pressing head. The rotating shaft may be connected to one side of the support member and may be connected to a surface of the pressing head such that the pressing head rotates around an external axis.

### 8.4.3 3-2 Embodiment: Kit+Pressing Head+First and Second Motors

In this regard, some embodiments of a device for testing a specimen using the kit described above will be described.

According to an embodiment of the present disclosure, in the test device, the patch receiving member included in the kit is pressed by using a pressing head connected to a rotating shaft located on opposite sides to deliver the reagent included in the patch receiving member to the specimen.

The test device may control the pressing head to operate in a similar manner to the patch receiving block of the device according to the second embodiment. The test device may control the position and/or posture of the pressing head such that the obliquely accessing the patch receiving member by the pressing head to allow the patch receiving member to access the substrate obliquely is similar to the oblique contact of the patch receiving block to the specimen.

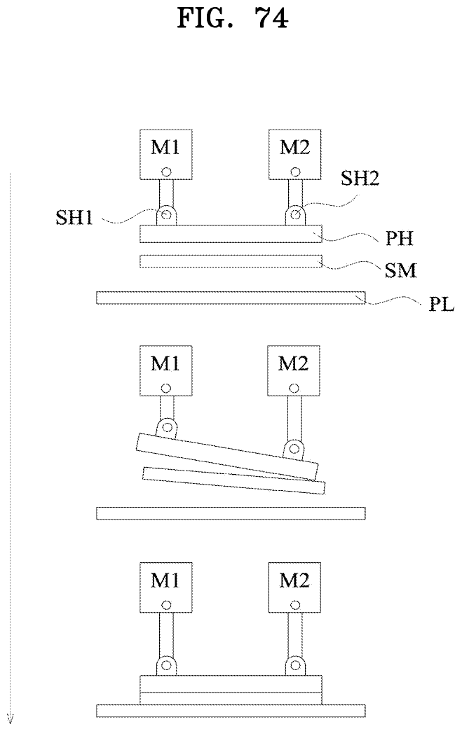

FIG. 74 illustrates an operation of a test device according to an embodiment of the present disclosure in the order of time. Referring to FIG. 74, the test device includes a motor M, a patch receiving member SM, a pressing head PH, a first rotating shaft, and a second rotating shaft, and changes the position and/or posture of the patch receiving member SM to test a specimen located on the substrate PL.

The present embodiment illustrated in FIG. 74 will be implemented in a similar manner to the description about the device in the second embodiment, unless defined otherwise.

Referring to FIG. 74, the test device may be prepared such that a bottom surface of the pressing head PH is to be parallel to the substrate PL or the patch receiving member SM.

Referring to FIG. 74, the test device drives the first motor M1 to lower the first rotating shaft SH1 so that one side (for example, right side) of the pressing head PH contacts the patch receiving member SM. The test device may lower the first rotating shaft SH1 such that the pressing head PH rotates by a certain distance in a counterclockwise direction with respect to the second rotating shaft SH2. The test device may lower the first rotating shaft SH1 connected to the pressing head PH such that the patch receiving member SM is pressed by the pressing head PH and thus one side (for example, the right side) is supported by the substrate PL.

Referring to FIG. 74, the test device drives the second motor M2 to lower the second rotating shaft SH2 so that another side (for example, the left side) of the pressing head PH contacts the patch receiving member SM. The test device may lower the second rotating shaft SH2 such that the pressing head PH rotates about the second rotating shaft SH2 by a certain distance. The test device may lower the second rotating shaft SH2 connected to the pressing head PH such that the patch receiving member SM is pressed by the pressing head PH and thus one side (for example, the right side) is supported by the substrate PL.

Referring to FIG. 74, as the pressing head PH lowers obliquely, one side (for example, the right side) of the patch receiving member SM accesses the substrate, and then, the other side (for example, the left side) thereof accesses the substrate. The posture of the patch receiving member SM may be changed such that the bottom surface thereof is parallel to the substrate PL as the second rotating shaft SH2 lowers. As the patch receiving member SM accesses the substrate PL from one side thereof and is then parallel to the substrate PL, the patch may sequentially contact the specimen from one side of the patch.

Although not illustrated in FIG. 74, the test device may space the patch receiving member SM from the substrate PL. The test device may drive the first motor M1 or the second motor M2 to raise the pressing head PH from one side of the pressing head PH. The test device may drive the second motor M2 to raise the pressing head PH from the right side of the pressing head PH. In this regard, the test device may control the driving of each of the first motor M1 and the second motor M2 such that with respect to the whole area of the sample, the time duration in which the sample is in contact with the patch is constant.

An embodiment of the present disclosure provides a patch control device to deliver a staining reagent used for staining to a specimen by using a gel-type patch which includes the staining reagent and a net structure forming micro-cavities for containing the staining reagent, and has a contact surface which is brought in contact with the specimen to deliver the staining reagent.

The patch control device may include a substrate fixing unit, a kit receiving unit, a pressing head, a driving portion, a first support member, and a second support member. The general configuration and operation of the substrate fixing unit, the driving portion, and the first and second support members may be implemented in a similar manner to that described in the first embodiment described above. The description of the kit receiving unit and the pressing head may be implemented in a similar manner to that described in the 3-1 embodiment described above, unless defined otherwise.

The first support member may be connected to the pressing head such that the support member extends from the driving portion in the Z-axis direction, may be connected, at an end thereof, to one side of the pressing head so that due to the driving force of the driving portion, the position of the side of the pressing head with respect to the Z axis is changed, and at the end, the pressing head freely rotates around the first rotating shaft in the X-axis direction perpendicular to the Z-axis direction.

The second support member may be connected to the pressing head such that the support member extends from the driving portion in the Z-axis direction, may be connected, at an end thereof, to another side opposite to the side of the pressing head so that due to the driving force of the driving portion, the position of the other side of the pressing head with respect to the Z axis is changed, and at the end, the pressing head freely rotates around the second rotating shaft in the X-axis direction perpendicular to the Z-axis direction.

The pressing head may be brought into contact with one side of the patch receiving member to provide a pressing force, so that as the driving portion is driven, the patch receiving member obliquely accesses the substrate on which the specimen is located to make the patch be in contact with the specimen from one side of the patch.

When the first support member lowers in the Z-axis direction by the driving portion, the pressing head may rotate about the second rotating shaft in a first direction so that a side of the pressing head is oblique toward the substrate on which the specimen is located. When the second support member lowers in the Z-axis direction by the driving portion, the pressing head may rotate about the first rotating shaft in a second direction so that another side of the pressing head is oblique toward the substrate on which the specimen is located and thus the contact surface of the patch is brought into contact with the patch receiving member from the side to the other side of the pressing member.

The driving portion may lower the first support member in the Z-axis direction such that, during the side of the pressing head is spaced from the substrate by a certain distance, the side of the pressing head is oblique to the substrate so that a side of the contact surface is brought into contact with the patch receiving member first and then another side thereof.

The driving portion may lower the second support member in the Z-axis direction such that, during the side of the pressing head is supported by the patch receiving member, the pressing head rotates about the first rotating shaft by a certain distance to allow the other side of the pressing head to access the substrate.

The driving portion may include a first driving portion and a second driving portion.

The pressing head may obliquely access the one side to the top surface of the patch receiving member according to the operation of the first driving portion. The oblique accessing, by the pressing head, the top surface of the patch receiving member may include bringing the side of the pressing head in contact with the patch receiving member when the pressing head rotates around the second rotating shaft as the first support member lowers in the Z-axis direction by the first driving portion.

The pressing head may contact the patch receiving member to press one side of the patch receiving member. The patch receiving member may access the substrate from the side pressed by the pressing head and bring one side of the patch into contact with the specimen.

The pressing head rotates around the first rotating shaft according to the operation of the second driving portion while the side thereof is in contact with the patch receiving member, and the rotating about the first rotating shaft by the pressing head may further include rotating following the top surface of the patch receiving member such that the other side thereof accesses the top surface of the patch receiving member.

The rotating the pressing head around the first rotating shaft may include bringing the pressing head to be in contact with the top surface of the patch receiving member sequentially to induce the rotation of the patch receiving member together with the pressing head.

The patch receiving member may rotate along the substrate such that as the pressing head rotates about the first rotating shaft, the patch is sequentially brought into contact with the specimen from one side of the patch.

The patch receiving member may rotate to be oblique with respect to the substrate according to the operation of the first driving portion, while the patch is in contact with the specimen.

The oblique rotation of the patch receiving member with respect to the substrate may include separating the patch receiving member from the substrate sequentially from one side to another side thereof so that the contact surface of the patch is separated from the specimen from one side to another side thereof as the first support member is raised in the Z-axis direction by the first driving portion.

When the first support member is raised in the Z-axis direction by the driving portion during the other side of the contact surface of the patch is in contact with the specimen, the pressing block may rotate around the second rotating shaft in the second direction such that the contact surface of the patch is separated from the specimen from the side to the other side of the contact surface of the patch.

The rotating around the second rotating shaft in the second direction may be controlling the rotation of the patch receiving member such that the contact time during which the specimen is in contact with the patch is constant in the whole area of the specimen.

When one side of the pressing head accesses the patch receiving member and the pressing head provides the pressing force to the patch receiving member, an edge of the side of the pressing head may be rounded to prevent the edge of the pressing head from being caught by the patch receiving member.

To prevent the pressing head from being caught by the patch receiving member when the pressing head is away from the substrate from the side thereof, an edge of another side of the pressing head may be rounded.

### 8.4.4 3-3 Embodiment: Kit+Pressing Head→Specific Operation

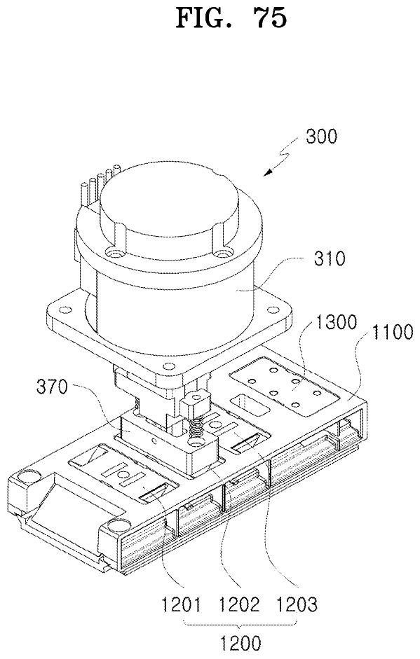

FIG. 75 illustrates a view of a test device according to an embodiment of the present disclosure. According to an embodiment of the present disclosure, provided is a test device for performing a test of a specimen by using a pressing head 370 that presses the test kit and elements which constitute the kit.

Referring to FIG. 75, the test device may include a motor 310 and the pressing head 370 driven by the motor 310. The test device may perform a test using a kit that includes a frame 1100, a patch receiving member 1200, and a medium receiving member 1300. Details of the kit may be implemented as described in the embodiments provided above.

The test device may move the frame 1100 in one direction.

The test device may smear the specimen on the substrate by moving the frame 1100 against the substrate placed in the base 1400. The test device may locate the medium receiving member 1300 on the smeared specimen by moving the frame 1100 against the substrate placed in the base 1400.

The test device may drive the motor 310 to cause the pressing head 370 to press the medium receiving member 1300.

The test device may change a receiving member located between a reaction area where the specimen is located and the pressing head 370, by moving the frame 1100 against a substrate placed in the base 1400.

The test device may drive the motor 310 to cause the pressing head 370 to press the patch receiving member 1200 couple to the kit. The test device may drive the motor 310 to press the patch receiving member 1200 in the order arranged.

On the other hand, in FIG. 75, it is described based on the case where the test device performs a function by moving the frame 1100 with respect to the base 1400. However, the test device may perform the same function by moving the base 1400 and/or motor 310 with respect to the frame 1100.

In addition, the shape of the pressing head 370 illustrated in FIG. 75 is only an example, and according to the disclosure disclosed in the present specification, the shape and operation of the pressing head 370 may vary as described in 3-1 embodiment, 3-2 embodiment, and 4 embodiment to be described later.

### 8.5 Fourth Embodiment: Damper

### 8.5.1 4-1 Embodiment: Damping Only

The test device according to an embodiment of the present disclosure may include a buffer structure that prevents the patch receiving member from excessively pressing the substrate or the specimen when pressing the patch receiving member using the pressing head disclosed in the present specification.

Meanwhile, according to an embodiment of the present disclosure, even when the test device delivers a reagent to a specimen by using the patch receiving block, other than the case where the test device presses the patch receiving member by using the pressing head, the test device may be implemented to include the buffer structure described above not to excessively press the specimen or the substrate by the patch receiving block.

However, for convenience of explanation, the following description is based on a case where the test device presses the patch receiving member using the pressing head and the patch receiving member accesses the substrate to deliver the reagent to the specimen.

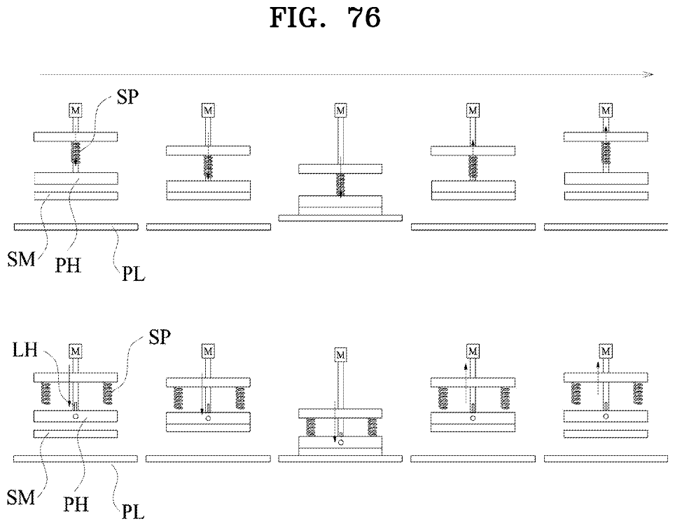

FIG. 76 illustrates an operation of a test device according to an embodiment of the present disclosure in the order of time. Referring to FIG. 76, the test device may include a motor M, a patch receiving member SM, a pressing head PH, an auxiliary member, and a spring SP connected to opposite sides of the auxiliary member. The auxiliary member may behave integrally with a support member connecting the pressing head PH with the motor M.

Referring to FIG. 76, in the test device, the pressing head PH may be positioned above and spaced from the patch receiving member SM by a certain distance. In this regard, the spring SP connected to the auxiliary member may be provided without contact with the pressing head PH.

The test device may lower the pressing head PH. The test device may lower the pressing head PH connected to the support member by lowering the support member.

The test device may lower the pressing head PH such that the pressing head PH contacts with the patch receiving member SM and the pressing head PH and the patch receiving member SM lower together.

The test device may lower the pressing head PH such that the pressing head PH and the patch receiving member SM lower and thus the contact surface of the patch exposed by the bottom surface of the patch receiving member SM is brought into contact with the specimen located on the substrate PL.

The test device may include a spring SP connected to the auxiliary member and a long hole LH formed in the support member, as a buffer structure that prevents applying of excess pressure to the patch and the specimen when the pressing head PH lowers to bring the contact surface of the patch into contact with the specimen located on the substrate PL.

The pressing head PH may be connected to the support member by a rod that passes through the long hole LH formed in the support member. The rod may move along the length direction of the long hole LH within the long hole LH.

The pressing head PH may move relative to the support member when the support member lowers while the pressing head PH and the patch receiving member SM are supported by the substrate PL. The pressing head PH may move relative to the support member such that when the support member lowers while being supported by the substrate PL, the rod may move upward in the long hole LH formed in the support member. The support member may lower further while the pressing head PH and the patch receiving member SM are supported on the substrate PL.

The support member may further lower to bring the spring SP into contact with the pressing head PH, while the pressing head PH and the patch receiving member SM are supported by the substrate PL. The test device may operate such that since the pressing head PH is supported by the spring SP, the pressing force incurring as the support member lowers does not directly act on the patch receiving member SM.

The spring SP may exert an elastic force, for example, and compressive force against the pressing head PH by being in contact with the pressing head PH while the pressing head PH is supported by the substrate PL through the patch receiving member SM.

The test device may raise the pressing head PH to separate the patch from the specimen. The test device may raise the pressing head PH to raise the patch receiving member SM by raising the support member.

In the case of using the test device according to the above embodiment, while the pressing head PH and the patch receiving member SM are supported by the substrate PL, the pressing force generated as the support member lowers is not directly delivered to the pressing head PH. Accordingly, the applying of excess pressure on the specimen and/or the patch may be prevented.

In addition, in the case of using the test device according to the above embodiment, it may be prevented from deteriorating the accuracy of the test result due to damage to the specimen and/or patch as excess pressure is applied to the specimen and/or patch.

### 8.5.2 4-2 Embodiment: Damping+Oblique Contact/Separation

According to an embodiment of the present disclosure, the test device may sequentially bring the patch into contact with the specimen from one side to another side thereof so that the reagent is uniformly delivered to the specimen, even in the case of including the buffer structure described above. Hereinafter, some embodiments of a test device in which the buffer structure described above is included and the contact surface of the patch sequentially contacts the specimen will be described.

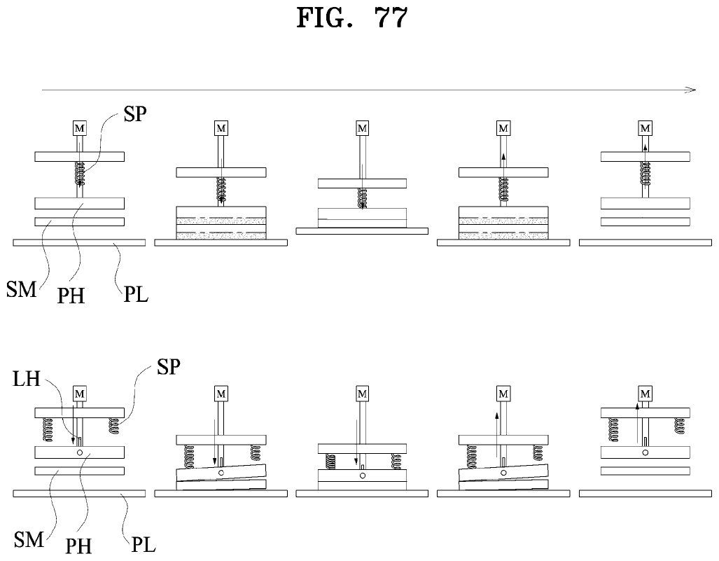

FIG. 77 illustrates an operation of a test device according to an embodiment of the present disclosure in the order of time. Referring to FIG. 77, the test device may include a motor M, a patch receiving member SM, a pressing head PH, an auxiliary member, and a spring SP connected to opposite sides of the auxiliary member. The motor M, the patch receiving member SM, the pressing head PH, and the spring SP, which constitute the test device may be implemented in a similar method to the description according to 4-1 embodiment.

Referring to FIG. 77, in the test device illustrated in FIG. 77, spring SP connected to opposite sides of the auxiliary member have different lengths. The test device may control the patch receiving member SM to obliquely access the substrate PL by using the springs SP having different lengths, so that the patch sequentially access the patch from one side to the other side to the specimen.

Referring to FIG. 77, in the test device, the pressing head PH may be positioned above and spaced from the patch receiving member SM by a certain distance. In this regard, the springs SP connected to the auxiliary member may be provided without contacting the pressing head PH. The springs SP may be arranged such that an end of each of the springs SP is fixed to the auxiliary member and other ends of the springs SP are spaced from the pressing head PH or the substrate PL by different distances.

The test device may lower the pressing head PH. The test device may lower the pressing head PH in a similar manner to the method described in the previous embodiment. The test device may include, as a buffer structure, the springs SP which are connected to opposite sides of the auxiliary member and have different lengths and a long hole LH formed in the support member.

When the support member lowers by the driving of the motor M, the position of the pressing head PH relative to the support member may be changed. The pressing head PH may move relative to the support member such that when the support member lowers while being supported by the substrate PL, the rod may move upward in the long hole LH formed in the support member. Accordingly, the distance between the auxiliary member and the pressing head PH may be reduced.

The test device may lower the support member such that the longer one of the springs SP contacts the pressing head PH. The test device may lower the support member such that, as the rod of the pressing head PH moves into a top portion of the long hole LH, the longer one of the springs SP is brought into contact with the pressing head PH.

The test device may lower the support member while the longer one of the springs SP is in contact with the pressing head PH. As the support member lowers, the pressing head PH may access the substrate PL from one side (for example, the left side) thereof which has been in contact with the spring SP. As the support member lowers, the pressing head PH may be changed to have a posture oblique to the substrate PL.

The test device may lower the support member such that one side (for example, the left side) of each of the pressing head PH and the patch receiving member SM is supported by the substrate PL.

The test device may lower the support member such that while one side of each of the pressing head PH and the patch receiving member SM is supported by the substrate PL, the pressing head PH and the patch receiving member SM rotate to bring the patch to be in contact with the specimen. As the support member lowers, the shorter one of the springs SP may contact the pressing head PH. Accordingly, the patch may contact the specimen sequentially.

The test device may operate such that since the pressing head PH is supported by the spring SP, the pressing force incurring as the support member lowers does not directly act on the patch receiving member SM.

The spring SP may exert an elastic force, for example, and compressive force against the pressing head PH by being in contact with the pressing head PH while the pressing head PH is supported by the substrate PL through the patch receiving member SM.

The test device may raise the pressing head PH to separate the patch from the specimen. The test device may raise the pressing head PH to raise the patch receiving member SM by raising the support member.

As the support member raises, one end of each of the springs SP attached to the auxiliary member may raise. The other end of the shorter one of the springs SP is separated from the pressing head PH and then, the longer one of the springs SP may be separated therefrom. The pressing head PH may be spaced away from the substrate PL from the side in contact with the short spring SP (for example, the right side). As the pressing head PH raises, the patch receiving member SM may be spaced from the substrate PL from one side (for example, the right side) of the patch receiving member SM, and the patch may be sequentially separated from the specimen from the side.

In the case of using the test device according to the above embodiment, in addition to the effect according to the test device described in the 4-1 embodiment described above, the reagent contained in the patch may be evenly delivered to the specimen by bringing the patch to be in oblique contact with the specimen. In addition, according to the present embodiment, damage to the specimen to be tested may be minimized by separating the patch obliquely from the specimen.

### 8.5.3 Examples of Device

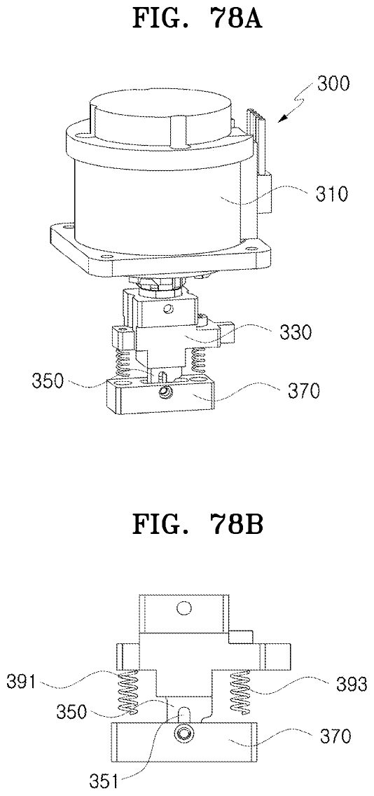

FIG. 78 illustrates a specific example of a test device according to an embodiment of the present disclosure. Referring to FIG. 78, the test device 300 according to the present disclosure may include a motor 310, an auxiliary member 330, a support member 350, and a pressing head 370.

(a) of FIG. 78 illustrates a portion of the test device 300 including the motor 310. (b) of FIG. 78 illustrates a portion of the test device 300 connected to the pressing head 370.

Referring to FIG. 78, the test device 300 may lower the support member 350 by using the motor 310. The support member 350 may lower together with the auxiliary member 330. The test device 300 may lower the support member 350 and the auxiliary member 330 to lower the pressing head 370.

A first spring 391 and a second spring 393 may be connected to opposite sides of the auxiliary member 330, respectively. The first spring 391 and the second spring 393 may come into contact with the pressing head 370 depending on the condition. The bottom end of each of the first spring 391 and the second spring 393 may come into contact with the pressing head 370 as the auxiliary member 330 lowers while the bottom surface of the pressing head 370 is supported.

A long hole 351 may be formed in the support member 350. A rod that supports the pressing head 370 and functions as a rotating shaft of the pressing head 370 may be coupled to the long hole 351. The rod may pass through the long hole 351.

The rod may move from a first position of the long hole 351 to a second position of the long hole 351 as the auxiliary member 330 lowers while the bottom surface of the pressing head 370 is supported. The second position may be closer to the top end of the long hole 351 than the first position.

A spring contact portion may be formed on the pressing head 370. The spring contact portion may contact the spring and support the end of the spring depending on the condition. The spring contact portion may be formed concave to the inside of the pressing head 370.

### 8.6 Fifth Embodiment: Medium Receiving Member

According to an embodiment of the present disclosure, a medium receiving member may be provided to receive a storage medium carrying a reagent used for a test and deliver the reagent to the specimen. The medium receiving member may not directly bring the storage medium to be in contact with the specimen, and may provide the reagent released from the storage medium to the specimen.

The medium receiving member may include a top plate and a pillar.

The top plate may be provided in the form of a flat plate. A pressing force may be applied to the top plate. The top plate may be formed with a protrusion to prevent the medium receiving member from being disengaged from the frame.

There are a plurality of pillars. The pillars may be formed to protrude from the top plate in a direction perpendicular to the top plate. Each of the pillars may be provided with a cylindrical shape having rounded edges. The respective pillars may be provided in the form of a cylinder having a thin end.

The pillars may include a first pillar and a second pillar disposed adjacent to each other. The first pillar and the second pillar may be spaced from each other by a predetermined distance. The pillars may further include a third pillar adjacent to the first pillar. The distance between the first pillar and the second pillar may be equal to the distance between the first pillar and the third pillar.

The storage medium may include a plurality of through-holes arranged to correspond to the pillars. The storage medium may be coupled to the medium receiving member so that the pillars pass through the through-holes, respectively.

The storage medium may include an elastic material. The storage medium may include a material with a restoring force. For example, the storage medium may be provided as a sponge.

The storage medium may carry a reagent used for testing the specimen. The storage medium may be used to carry a large amount of reagent required for testing the specimen.

For example, the storage medium may carry a fixation solution to fix the specimen. The storage medium may be compressed by an external force. The storage medium may be compressed by external force to release the reagent.

The storage medium may expand. When the external force is removed while the storage medium is compressed by external force, the storage medium may expand and restore to its original shape. The storage medium may expand to its original form and reabsorb the reagent.

The medium receiving member may be coupled into the test kit described above. The medium receiving member may be coupled to the frame of the test kit. The medium receiving member may, depending on the condition, be coupled to the frame such that the storage medium is positioned inside the frame. The medium receiving member may, depending on the condition, be coupled to the frame such that ends of the pillars protrude the frame and thus are outside thereof.

The frame may include a plurality of holes formed to correspond to the pillars. The holes may be formed in the lower surface of the medium receiving member housing portion for housing the medium receiving member of the frame. Each of the holes may have a greater diameter than the outer diameter of the corresponding pillar of the pillars.

The medium receiving member may be coupled to the storage medium, and may be coupled to the frame such that the pillars face the substrate on which the specimen is located. The medium receiving member may be prepared such that ends of the pillars are to be exposed through the holes of the frame.

When a pressing force is applied to the top plate, the medium receiving member may be positioned in the lowering position within the frame. When a pressing force is applied to the top plate, the medium receiving member may lower within the frame.

When a pressing force is applied to the top plate, the storage medium may be in a compressed state. When a pressing force is applied to the top plate, the storage medium may be compressed.

When the storage medium is compressed, the reagent carried in the storage medium may be released from the storage medium. When the storage medium is compressed, the reagent released from the storage medium may remain connected to the storage medium.

When the storage medium is compressed, the reagent released from the storage medium may leak out of the frame through a gap between a plurality of holes and a plurality of pillars that pass through a plurality of holes. The reagent leaking out of the frame may move along the outer surfaces of a plurality of pillars.

The medium receiving member may have an end which is located adjacent to the specimen so that the released reagent moves along the outer surfaces of the pillars and is delivered to the specimen. The medium receiving member may have an end which is located adjacent to the specimen so that the reagent moves along the outer surfaces of the pillars to merge on the substrate. The reagent may move along the outer surface of each of the pillars and merge on the substrate to form a reagent layer. The reagent layer may cover the test area of the specimen. The released reagent may be maintained connected to the storage medium or a reagent in the storage medium.

The released reagent may be maintained connected to the storage medium or the reagent in the storage medium by surface tension.

When the pressing force on the top plate is removed, the medium receiving member may be positioned in the raising position within the frame. The medium receiving member may be raised within the frame when the pressing force on the top plate is removed.

When the pressing force on the top plate is removed, the storage medium may expand. The storage medium may expand and restore to its original shape.

When the storage medium expands, the storage medium may reabsorb at least a portion of the reagent released outside the frame. When the storage medium expands, the storage medium may reabsorb the reagent which has been released out of the frame and connected to the storage medium.

The function of the medium receiving member described above may be performed by the test device disclosed in the present specification. The test device according to an embodiment of the present disclosure may house the kit and/or medium receiving member described above, and may provide a reagent to the specimen located on the substrate by applying the pressing force on the top plate.

When a specimen is tested by using a medium receiving member according to the present disclosure and a kit including the same, the amount of reagent consumed in the test of the specimen may be reduced. At the same time, the amount required for the test of the specimen may be sufficiently supplied to the specimen, leading to high test efficiency.

In detail, in order to fix the specimen, the specimen must be sufficiently soaked in the fixing solution. Accordingly, in general, a large amount of the fixing solution is poured onto a substrate on which the specimen is located, and then dried. Accordingly, even in the case of fixing one sample, a large amount of the fixing solution needs to be consumed. In contrast, when the fixing solution is delivered by using the medium receiving member disclosed in the present specification, the amount of fixing solution consumed is significantly less compared to the method of the related art while the fixing solution is sufficiently delivered to the specimen. Accordingly, the test may be performed effectively.

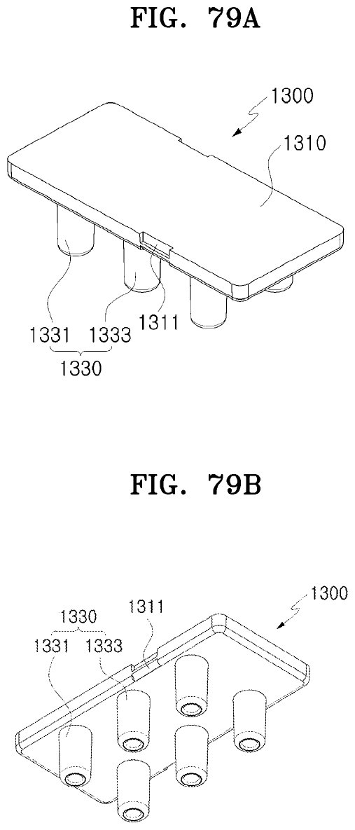

FIG. 79 illustrates an example of a medium receiving member 1300 according to an embodiment of the present disclosure. Referring to (a) and (b) of FIG. 79, the medium receiving member 1300 may include a top plate 1310 and a plurality of pillars 1330.

The medium receiving member 1300 may be coupled to the frame 1100 disclosed in the present specification as illustrated in FIG. 71. Hereinafter, with reference to FIG. 71, the medium receiving member 1300 illustrated in FIG. 79 will be described.

The top plate (1310) may have a recess 1311. The recess 1311 may be coupled to the protrusion of the frame to fix the medium receiving member 1300 without the disengagement of the medium receiving member 1300 from the frame. The recess 1311 may limit the range of movement of the medium receiving member 1300 with respect to the frame.

The pillars 1330 may be arranged to have certain intervals. The pillars 1330 may include a first pillar 1331 and a second pillar 1333. The first pillar 1331 and the second pillar 1333 may be spaced apart from each other by a predetermined interval.

Each pillar of the pillars 1330 may have a thin cylindrical end.

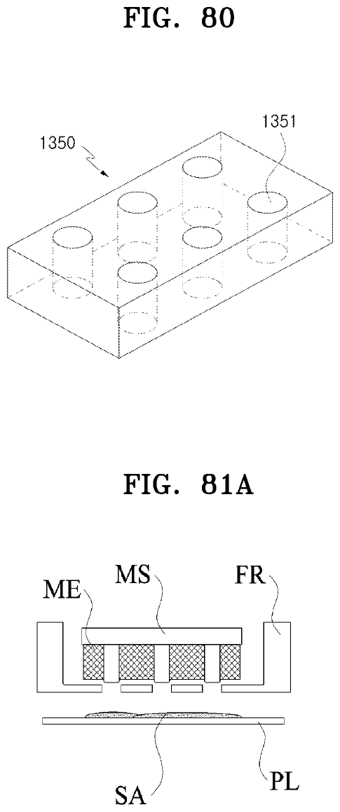

FIG. 80 illustrates a view of a storage medium 1350 according to an embodiment of the present disclosure.

The storage medium 1350 may include a plurality of through-holes 1351 formed to correspond to a plurality of pillars formed in the medium receiving member described above. The storage medium 1350 may be coupled to the medium receiving member such that the pillars pass through the through-holes 1351.

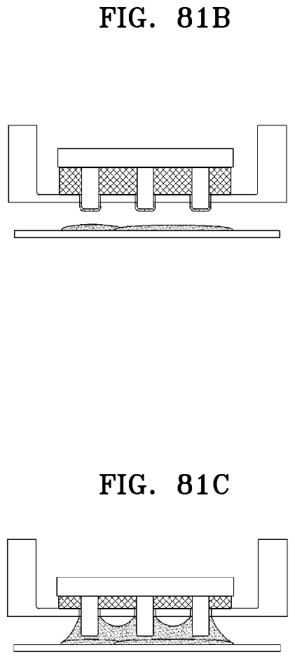

FIG. 81 schematically illustrates a test method according to an embodiment of the present disclosure in the order of time. In detail, FIG. 81 briefly illustrates a method of providing a reagent to the specimen SA located on the substrate PL by using the storage medium disclosed in the present specification.

Referring to FIG. 81, the test method according to an embodiment of the present disclosure may be performed by using a kit including a frame FR, a medium receiving member MS, and a storage medium ME.

Referring to (a) of FIG. 81, the test method according to an embodiment of the present disclosure may include preparing the kit on the substrate PL on which the specimen SA is located. The kit may be prepared such that a bottom surface of the frame FR faces the substrate PL. The bottom surface of the frame FR may be one surface having a plurality of holes therein.

The medium receiving member MS may be placed inside the frame FR. The medium receiving member MS is placed in the frame FR such that the pillars are exposed to the outside of the frame FR through the holes.

Referring to (a) of FIG. 81, the test method may include preparing the kit in a standby state in which the storage medium ME is not compressed. In the standby state, a pressing force may not act on the top plate of the medium receiving member MS. In the standby state, the end of each of the pillars may be positioned inside the frame FR. In the standby state, the end of each of the pillars may protrude inside the frame FR.

The storage medium ME may be prepared as carrying the reagent and not being compressed. The storage medium ME may be coupled to the medium receiving member MS.

Referring to (b) of FIG. 81, the test method may include lowering the medium receiving member MS described above. The test method may include lowering the medium receiving member MS so that the storage medium ME is compressed. The test method may include lowering the medium receiving member MS by applying a pressing force to the top plate of the medium receiving member MS toward the substrate PL.

When the medium receiving member MS lowers, the ends of the pillars may protrude out of the frame FR. When the medium receiving member MS lowers, the storage medium ME may release the reagent. The released reagent may leak to the outside of the frame FR along the outer surfaces of the pillars.

Referring to (c) of FIG. 81, the test method may include further lowering of the medium receiving member MS described above. The test method may include lowering the medium receiving member MS such that an end thereof is adjacent to the specimen. The test method may include lowering the medium receiving member MS such that the end thereof is adjacent to the specimen and the reagent contacts the specimen.

Referring to (c) of FIG. 81, the test method may further include changing the kit to a release state in which the storage medium ME is compressed. In the release state, the ends of the pillars may protrude to the outside of the frame FR. In the release state, the ends of the pillars may be downward closer to the substrate than in the standby state. In the release state, the ends of the pillars may be located adjacent to the substrate so that the reagent released from the storage medium ME contacts the specimen as the storage medium ME is compressed.

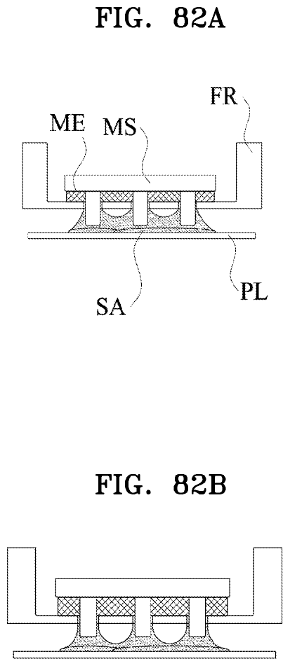

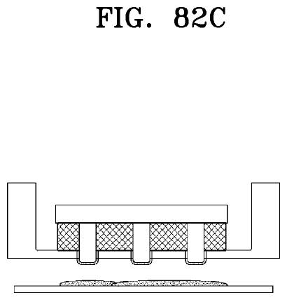

FIG. 82 schematically illustrates a test method according to an embodiment of the present disclosure in the order of time. In detail, (a), (b), and (c) of FIG. 82 briefly illustrates a method of absorbing a reagent provided to the specimen SA located on the substrate PL by using the storage medium disclosed in the present specification.

Referring to FIG. 82, the test method according to an embodiment of the present disclosure may be performed by using a kit including a frame FR, a medium receiving member MS, and a storage medium ME.

Referring to (a) of FIG. 82, the test method according to an embodiment of the present disclosure may include providing a reagent to the specimen SA by using the kit. Referring to (a) of FIG. 82, the test method may include preparing the release state. The test method illustrated in (a) of FIG. 82 may be embodied in a similar manner to the method illustrated in connection with (c) of FIG. 81.

Referring to (b) of FIG. 82, the test method according to an embodiment of the present disclosure may include raising the medium receiving member MS. The test method may include raising the medium receiving member MS by reducing at least a portion of the force applied to the top plate of the medium receiving member MS.

When the medium receiving member MS is raised, the storage medium ME may expand due to a restoring force. In detail, as the force decreases, the storage medium ME expands, and the medium receiving member MS may be raised due to the restoring force of the storage medium ME.

When the storage medium ME expands, negative pressure occurs in the storage medium ME and thus, at least a portion of the released reagent may be absorbed by the storage medium ME. When the storage medium ME expands, at least a portion of the released reagent connected to the storage medium ME or the reagent carried on the storage medium ME may be reabsorbed by the storage medium ME.

Referring to (c) of FIG. 82, the test method may further include changing the kit to the standby state described above.

Referring to (c) of FIG. 82, the test method may raise the medium receiving member MS so that the storage medium ME or the reagent carried on the storage medium ME is separated from the specimen. In the test method, the pressing force applied to the top plate of the medium receiving member MS is reduced and thus the storage medium ME expands, and, as the pillars are raised, the medium receiving member MS and the reagent carried by the medium receiving member MS may be separated from the specimen. When the medium receiving member MS is raised, the reagent released by the specimen may be removed. Alternatively, a trace amount of the released reagent may remain in the specimen.

Meanwhile, although not illustrated in FIGS. 81 and 82, the frame may further include a medium receiving member support. The medium receiving member support may support the bottom surface of the storage medium, as described above with respect to FIG. 68. The medium receiving member support may support opposite sides of the top plate, limiting the range of lowering of the medium receiving member.

According to an embodiment of the present disclosure, a test kit including the medium receiving member described above may be provided.

The test kit may include a porous storage medium which carries the reagent used for the test, is compressed by an external force to release at least a portion of the reagent, and has a plurality of through-holes, a top plate having a top surface on which a pressing force acts, a medium receiving member which has a top plate having a top surface on which a pressing force acts and a plurality of pillars extending from a bottom surface of the top plate in a direction perpendicular to the top plate to provide a movement path of the released reagent, and is coupled to the storage medium in such a way that the pillars pass through the through-holes, and a frame which communicates the through-holes and receives the medium receiving member in such a way that the pillars are movable between a first position and a second position in the direction in which the pillars extend.

The frame may be positioned to have a bottom surface which faces a substrate on which the specimen is located. The frame may include steps that are symmetrically formed on opposite sides of the holes to limit the movement range of the medium receiving member.

The medium receiving member may receive the storage medium to be positioned between the top plate and the bottom surface of the frame.

Each hole included in the holes may have an inner diameter greater than the inner diameter of each through-hole included in the through-holes.

The pillars may be provided in the form of a cylinder. Two adjacent pillars of the pillars may be spaced from each other by a predetermined distance.

The reagent used for testing the specimen may be a fixing reagent to fix the specimen. The fixing reagent used for testing the specimen may be a solution including any one of formaldehyde, methanol, ethanol, picric acid, acetic acid, chromic acid, or glutaraldehyde.

When the medium receiving member is in the first position, the storage medium has a first height, and the pillars may be positioned inside the frame.

When the medium storage member is in the second position, the storage medium has a second height which is obtained by compressing at least a portion of the medium storage member having the first height, and the ends of the pillars may protrude to the outside of the frame through the pores.

When the medium receiving member is in the second position, a ring-shaped gap is formed between the outer surface of the pillars and the inner surface of the through-holes, and the gap may provide a movement path through which the reagent released from the compressed storage medium moves toward the outside of the frame.

The medium receiving member may move from the first position to the second position as the pressing force acts on the top surface of the top plate.

When the medium receiving member moves from the first position to the second position, the storage medium is compressed to release at least a portion of the reagent, and the pillars may provide the movement path through which the released reagent moves onto the substrate.

The reagent merges on the substrate to form a reagent layer, and the reagent layer may be connected to the storage medium through the reagent distributed on the respective outer surfaces of the pillars.

The medium receiving member may move from the second position to the first position as the pressing force acting on the top surface of the top plate is removed. The storage medium has an elastic property and may exert a restoring force on the medium receiving member as the pressing force acting on the top surface is removed.

When the medium receiving member moves from the second position to the first position, the storage medium expands to absorb at least a portion of the released reagent, and the pillars may provide a movement path to allow the released reagent to move toward the storage medium.

When the medium receiving member is in the second position, the ends of the pillars may be located adjacent to the substrate so that the reagent released from the storage medium moves along the respective outer surfaces of the pillars and merges on the substrate.

The ends of the pillars may contact the specimen. The ends of the pillars do not contact the specimen and may be located adjacent to the specimen.

According to an embodiment of the present disclosure, provided is a medium receiving member which is coupled to a porous storage medium to test a specimen, wherein the porous storage medium carries a reagent used for the test, is compressed by an external force to release at least a portion of the reagent, and has a plurality of through-holes.

The medium receiving member may have a top plate on which a pressing force acts and a plurality of pillars extending from the bottom surface of the top plate in a direction perpendicular to the top plate to provide a movement path for the released reagent.

The medium receiving member may be placed to be movable along the direction in which the pillars extend between the first position and the second position in a frame having a plurality of pores communicating the through-holes, wherein the frame has a bottom surfaces facing a substrate on which the specimen is located.

The medium receiving member may be coupled to the storage medium such that the pillars pass through the through-hole and the storage medium is located between the top plate and the bottom surface of the frame.

When the medium receiving member is in the first position, the storage medium has a first height, and the pillars may be positioned inside the frame.

When the medium storage member is in the second position, the storage medium has a second height which is obtained by compressing at least a portion of the medium storage member having the first height, and the ends of the pillars may protrude to the outside of the frame through the pores.

When the medium receiving member moves from the first position to the second position, the storage medium is compressed to release at least a portion of the reagent, and the pillars may provide the movement path through which the released reagent moves onto the substrate.

The frame includes a step symmetrically formed with opposite sides of each of the holes to limit the movement range of the medium receiving member, and when the medium receiving member is in the second position, opposite sides of the top plate may be supported by the step.

When the medium receiving member is in the second position, a ring-shaped gap is formed between the outer surface of the pillars and the inner surface of the through-holes, and the gap may provide a movement path through which the reagent released from the compressed storage medium moves toward the outside of the frame.

Each hole included in the holes may have an inner diameter greater than the inner diameter of each through-hole included in the through-holes.

When the medium receiving member moves from the second position to the first position, the storage medium expands to absorb at least a portion of the released reagent, and the pillars may provide a movement path to allow the released reagent to move toward the storage medium.

The medium receiving member may move from the first position to the second position as the pressing force acts on the top surface of the top plate.

The storage medium has an elastic property and may exert a restoring force on the medium receiving member as the pressing force acting on the top surface is removed.

When the medium receiving member is in the second position, the ends of the pillars may be located adjacent to the substrate so that the reagent released from the storage medium moves along the respective outer surfaces of the pillars and merges on the substrate.

The reagent merges on the substrate to form a reagent layer, and the reagent layer may be connected to the storage medium through the reagent distributed on the respective outer surfaces of the pillars.

The reagent used for testing the specimen may be a fixing reagent to fix the specimen, and the fixing reagent may be a solution including any one of formaldehyde, methanol, ethanol, picric acid, acetic acid, chromic acid, or glutaraldehyde. The reagent used for the present disclosure is not limited thereto, and various kinds of reagents may be used to fix the specimen in performing a test according to the present disclosure.

The pillars may be provided in the form of a cylinder.

The top plate may further include a protrusion that prevents the medium receiving member from disengaging from the frame.

According to an embodiment specimen of the present disclosure, a kit to be used for testing may be provided. The kit may include a frame to be coupled to a substrate on which the specimen is located, a patch receiving member that is coupled to the frame, places a patch containing a staining reagent for staining the specimen in such a way that one surface of the patch is exposed to the outside the frame, and brings the patch into contact with the specimen to deliver the staining reagent, and a medium receiving member which has a top plate and a plurality of pillars extending from the top plate, and is coupled to a storage medium, which contains a fixing reagent for fixing the specimen, and is placed in the frame such that the storage medium is located between the top plate and the bottom surface of the frame, wherein the fixing reagent is released to the outside of the frame and is delivered to the specimen.

The patch receiving member and the medium receiving member may be arranged in the frame in the length direction of the substrate.

The frame may slide relative to the substrate.

The frame may further include a smear film housing portion having an inclined surface on which a smear film for smearing the specimen on the substrate is located.

The medium receiving member, the patch receiving member, and the smear film housing portion may be arranged in the frame in the length direction of the substrate.

The frame may slide such that the specimen is smeared, by the smear film, on the substrate in the length direction of the substrate.

The frame may slide such that the patch receiving member is located on a test area of the substrate in which the specimen is located.

The frame may slide such that the medium receiving member is located on a test area of the substrate in which the specimen is located.

The frame may include a sliding rail. The test kit may further include a base formed with guide rails coupled to the sliding rail to provide a movement path for the sliding rail.

The guide rails may be formed on opposite sides of the base. The base may further include a window and a substrate receiving portion provided between the guide rails.

The medium receiving member may include a top plate on which a pressing force acts and a plurality of pillars extending from the bottom surface of the top plate in a direction perpendicular to the top plate to provide a movement path for the released reagent, and the medium receiving member may place the storage medium such that the storage medium is located between the top plate and the bottom surface of the frame.

The storage medium is compressed by an external force to release at least a portion of the reagent, and has a plurality of through-holes and thus, is coupled to the medium receiving member such that the pillars pass through the through-holes.

The frame may communicate with the through-holes and may place the medium receiving member such that the medium receiving member is movable between the first position and the second position in the direction in which the pillars extend.

When the medium receiving member is in the first position, the storage medium has a first height, and the pillars may be positioned inside the frame.

When the medium storage member is in the second position, the storage medium has a second height which is obtained by compressing at least a portion of the medium storage member having the first height, and the ends of the pillars may protrude to the outside of the frame through the pores.

The frame may place the patch receiving member such that the patch receiving member moves up and down between a third position and a fourth position.

When the patch receiving member is in the third position, the patch is separated with the specimen, and when the patch receiving member is in the fourth position, the patch may contact the specimen.

According to an embodiment of the present disclosure, a method of testing a specimen is provided, the method using a medium receiving member having a top plate and a plurality of pillars extending from a bottom surface of the top plate in a direction perpendicular to the top plate, a porous storage medium which carries a reagent used for testing the specimen, having a plurality of through-holes arranged to correspond to the pillars, and is coupled to the medium receiving member such that the pillars pass through the through-holes, and a frame which has a plurality of pores arranged to correspond to the pillars, and receives the medium receiving member to be movable between the first position and the second position.

The test method may include placing a bottom surface of the frame to face the surface of a substrate on which the specimen is located, at a position where the kit in which the medium receiving member is located in the first position is spaced from the substrate on which the specimen is located by a certain distance. When the medium receiving member is in the first position, the storage medium has a first height, and the pillars may be positioned inside the frame.

The test method may include moving the medium receiving member from the first position to the second position so that at least a portion of the storage medium is compressed and the ends of the pillars protrude to the outside of the frame through the holes. When the medium storage member is in the second position, the storage medium has a second height which is obtained by compressing at least a portion of the medium storage member having the first height, and the ends of the pillars may protrude to the outside of the frame through the pores.

The test method may include placing the ends of the pillars to be adjacent to the specimen such that the reagent released from the storage medium leaks along the outer surface of the pillars and merges onto the substrate to provide to the specimen.

The test method may further include moving the medium receiving member from the second position to the first position so that the reagent provided to the specimen is reabsorbed into the storage medium.

The placing the ends of the pillars to be adjacent to the specimen may further include placing the ends to be adjacent to the specimen such that the reagent leaked to the outside of the frame is merged onto the substrate to form a reagent layer, and the reagent layer is connected to the storage medium through the reagent distributed on the outer surface of each of the pillars.

The kit may further include a patch receiving member to receive a patch that contains a staining reagent for staining the specimen. The frame may receive the patch receiving member to be movable upward or downward between the third position and the fourth position. When the patch receiving member is in the third position, the patch is separated with the specimen, and when the patch receiving member is in the fourth position, the patch may contact the specimen.

The preparing the kit may further include preparing a kit in which the patch receiving member is located in the third position. The test method may further include moving the patch receiving member from the third position to the fourth position so that the patch contacts the specimen and the reagent is provided to the specimen.

The frame may further include a smear film housing portion having an inclined surface on which a smear film for smearing the specimen on the substrate is located.

After the preparing the kit, the test method may further include sliding the frame against the substrate to smear the specimen on the substrate by using the smear film in the length direction of the substrate.

The above description is provided for an illustrative purpose only, and those of ordinary skill in the art to which the present invention pertains will be able to make various modifications and variations without departing from the essential characteristics of the present disclosure.

Accordingly, the embodiments of the present disclosure described above may be implemented separately or in combination with each other.

Accordingly, the embodiments disclosed in the present disclosure are not intended to limit the technical idea of the present disclosure, but are for descriptive purposes, and the scope of the technical idea of the present disclosure is not limited by these embodiments. The scope of protection of the present disclosure should be interpreted by the scope of the claims below, and all technical ideas within the scope of the present disclosure should be construed as being included in the scope of the present disclosure.

1-14. (canceled)

15. A device for controlling the posture of a patch to stain a specimen by using a gel-type patch which includes a staining reagent used to stain a specimen, a net structure forming micro-cavities containing the staining reagent, and a contact surface which is brought in contact with the specimen to deliver the staining reagent, the device comprising:
a substrate fixing unit to fix the substrate on which the specimen is located;
a kit receiving unit in which a kit including at least one patch receiving member is located, wherein the patch receiving member receives the patch such that at least a portion of the patch is exposed; and
a control unit controlling the position of the at least one patch receiving member relative to the substrate, wherein
the control unit positions the patch to be in a first posture, which is an oblique posture at which an end of the contact surface is closer to the surface of the substrate on which the specimen is placed than another end of the contact surface while the contact surface of the patch faces the surface of the substrate on which the specimen is placed, allows the patch to access the surface of the substrate while maintaining the patch to be at the first posture, and brings the patch to be in contact with the specimen placed on the surface of the substrate to deliver the staining reagent to the specimen.

16. The device of claim 15, wherein
the control unit controls the position of the patch receiving member relative to the surface of the substrate such that, at the first posture, an end of the patch contacts the specimen placed on the surface of the substrate, and when the posture of the patch is changed from the first posture to a second posture at which the contact surface is parallel to the surface of the substrate, the contact surface of the patch is fitted with the surface of the substrate and the contact area of the surface of the substrate in contact with the specimen extends in a first direction.

17. The device of claim 16, wherein
the controlling, by the control unit, the patch receiving member to bring an end of the patch at the first posture to be in contact with the specimen placed on the surface of the substrate, includes bringing the end of the patch to be in contact with the specimen to form an water film between the end in contact and the specimen, and
the changing the patch from the first posture to the second posture by the control unit includes changing the posture of the patch to the second posture so as to grow the water film formed between the end and the specimen in the first direction by allowing the contact surface to sequentially access the surface of the substrate from the end to the other end of the contact surface.

18. The device of claim 15, wherein
the control unit controls the posture of the patch receiving member to space the patch apart from the surface of the substrate, wherein
the controlling, by the control unit, the posture of the patch receiving member to space the patch from the surface of the substrate further includes controlling the posture of the patch receiving member such that the patch is changed from the second posture to a third posture in which the patch is oblique to the surface of the substrate so that at least a portion of the contact surface is separated from the specimen and the contact area is reduced in a second direction.

19. The device of claim 18, wherein
the second direction is a reverse direction of the first direction, and the third posture is the same as the first posture, and
the control unit controls the posture of the patch receiving member such that the patch is sequentially separated from the specimen from the other end to the end of the contact surface.

20. The device of claim 18, wherein
the second direction is parallel to the first direction, and
the control unit controls the posture of the patch receiving member such that the patch is sequentially separated from the specimen from the end to the other end of the contact surface.

21. The device of claim 18, wherein
the controlling, by the control unit, the posture of the patch receiving member to space the patch apart from the surface of the substrate further includes
controlling the posture of the patch receiving member such that, to uniformly deliver the staining reagent to the whole area of the specimen, the time during which the patch is in contact is maintained constant with respect to the whole area of the specimen.

22. The device of claim 15, wherein
the specimen is a smeared blood sample.

23. The device of claim 15, further comprising
a pressing head that contacts the patch receiving member and provides a pressing force to the patch receiving member, wherein
the controlling, by the control unit, the position of the patch receiving member relative to the substrate, includes controlling, by the control unit, the relative position of the patch receiving member which contacts the pressing head by controlling the position of pressing head by the pressing head.

24-29. (canceled)

30. A device for controlling the posture of a patch to deliver a staining reagent used for staining to a specimen by using a gel-type patch which includes the staining reagent and a net structure forming micro-cavities for containing the staining reagent, and has a contact surface, which is a surface to be brought into contact with the specimen, the device comprising:
a substrate fixing unit to fix the substrate on which the specimen is located;
a kit including at least one patch receiving member which receives the patch such that at least a portion of the contact surface is exposed to the outside, and
a control unit to control the position of the at least one patch receiving member with respect to the substrate, wherein
the control unit controls the posture of the at least one patch receiving member such that one side of the patch is brought in contact with the specimen, the contact surface is sequentially brought in contact with the specimen from the side to another of the patch to prevent the formation of air bubbles between the contact surface and the specimen, the staining reagent is delivered to the specimen through the contact surface of the patch, and the contact surface of the patch is sequentially separated from the side to the other side of the patch to uniformly deliver the staining reagent to an area in which the specimen is distributed.

31. The device of claim 30, wherein
the patch receiving member has a rounded edge, and
the control unit controls the posture of the at least one patch receiving member such that the at least one patch receiving member is allowed to access the substrate from the rounded edge of the at least one patch receiving member so as to minimize the friction between the at least one patch receiving member and the substrate or the specimen when the patch is sequentially brought into the specimen.

32. The device of claim 30, wherein
the controlling, by the control unit, the posture of the at least one patch receiving member to bring the contact surface into contact with the specimen sequentially further includes controlling the posture of the at least one patch receiving member such that the contact surface contacts the specimen from a side to another side of the patch at a first speed, and
the controlling, by the control unit, the posture of the at least one patch receiving member to separate the contact surface from the side to the other side of the patch further includes controlling the at least one patch receiving member such that the contact surface is separated from the specimen to separate the contact surface from the specimen at the first speed to uniformly deliver the staining reagent to the specimen.

33-34. (canceled)

35. The device of claim 30, further comprising
a pressing head that contacts the patch receiving member and provides a pressing force to the patch receiving member, wherein
the controlling, by the control unit, the position of the patch receiving member relative to the substrate, includes controlling, by the control unit, the relative position of the patch receiving member which contacts the pressing head by controlling the position of the patch receiving member by the pressing head.

36-47. (canceled)

48. A device for controlling a patch by using a gel-type patch which includes a staining reagent used to stain a specimen and a net structure forming micro-cavities for containing the staining reagent, and has a contact surface which is brought in contact with the specimen to deliver the staining reagent to the specimen, the device comprising:
a substrate fixing unit to fix the substrate on which the specimen is located;
a kit including at least one patch receiving member which receives the patch such that at least a portion of the contact surface is exposed to the outside, and
a control unit to control the posture of the at least one patch receiving member relative to the substrate, wherein
the control unit controls the posture of the at least one patch receiving member such that the patch is located in a first posture on one side of the surface of the substrate while the contact surface of the patch faces the surface of the substrate on which the specimen is placed, the patch accesses the surface of the substrate while retaining the first posture thereof, the posture of the patch is changed from the first posture to a second posture in which at least a portion of the contact surface contacts the specimen so that the patch contacts the specimen placed on the surface of the substrate, thereby delivering the staining reagent to the specimen, separating an end of the contact surface from the surface of the substrate, and then another end of the contact surface therefrom so that the patch is changed to a third posture in which the patch is positioned oblique to the surface of the substrate.

49. The device of claim 48, wherein
the controlling the posture of the patch receiving block to change the patch to be in the third posture further comprises
changing the patch from the second posture to the third posture to reduce the contact area, in which the contact surface contacts the specimen, in one direction.

50. The device of claim 48, wherein
the controlling, by the control unit, the posture of the patch receiving member to bring the patch to be in contact with the specimen comprises controlling the posture of the patch receiving member such that an water film is formed between the contact surface and the specimen, and
the controlling, by the control unit, the posture of the patch receiving member to change the patch to be in the third posture comprises controlling the posture of the patch receiving member such that, as the contact surface is sequentially separated from the specimen from the end to the other end of the contact surface, the water film formed between the contact surface and the specimen is reduced in one direction.

51. The device of claim 48, wherein
the controlling, by the control unit, the posture of the patch receiving member to change the patch to be in the first posture comprises controlling, by the control unit, the posture of the patch receiving member such that while the contact surface of the patch faces the surface of the substrate on which the specimen is placed, the patch is positioned in the first posture, that is, an oblique posture in which the end of the contact surface is closer to the surface of the substrate on which the specimen is placed than the other surface of the contact surface.

52. The device of claim 48, wherein
the controlling, by the control unit, the posture of the patch receiving member to bring the patch to be in contact with the specimen comprises controlling the posture of the patch receiving member such that the end of the contact surface is in contact with the specimen before the other end thereof, and as the patch is changed from the first posture to the second posture, the contact area in which the contact surface contacts the specimen extends from the end to the other end of the contact surface to bring the patch to be in contact with the specimen sequentially.

53. The device of claim 52, wherein
the controlling, by the control unit, the posture of the patch receiving member to space the patch apart from the surface of the substrate includes
controlling the posture of the patch receiving member such that the time taken for the contact area to extend from the end to the other end thereof is the same as the time taken for the contact area to be reduced from the end to the other end thereof to uniformly deliver the staining reagent to the whole area of the specimen.

54. The device of claim 48, wherein
the controlling, by the control unit, the posture of the patch receiving member to bring the patch to be in contact with the specimen comprises controlling the posture of the patch receiving member such that the patch is changed from the first posture to the second posture to bring the other end of the contact surface to be in contact with the specimen before the end thereof, and thus, the contact area in which the contact surface contacts the specimen extends from the other end to the end of the contact surface to bring the patch to be in contact with the specimen sequentially.

55. (canceled)
